(19) United States
(12) Patent Application Publication
(10) Pub. No.: US 2023/0346784 A1
(43) Pub. Date: Nov. 2, 2023

(54) METHOD FOR TREATING COMPLEMENT MEDIATED DISORDERS CAUSED BY BETACORONAVIRUSES

(71) Applicants: Alexion Pharmaceuticals, Inc., Boston, MA (US); The Johns Hopkins University, Baltimore, MD (US)

(72) Inventors: Moshe Vardi, Newton, MA (US); Robert A. Brodsky, Towson, MD (US); Xiang Gao, Guilford, CT (US); Shamsah D. Kazani, Watertown, MA (US); Venu Nagali, Columbus, NJ (US); Xuan Yuan, Kingsville, MD (US)

(73) Assignees: Alexion Pharmaceuticals, Inc., Boston, MA (US); The Johns Hopkins University, Baltimore, MD (US)

(21) Appl. No.: 18/022,330

(22) PCT Filed: Aug. 27, 2021
(86) PCT No.: PCT/US2021/047901

§ 371 Date: Feb. 21, 2023

Related U.S. Application Data
(60) Provisional application No. 63/071,547, filed on Aug. 28, 2020.
(60) Provisional application No. 63/159,089, filed on Mar. 10, 2021.

(51) Int. Cl. A61K 31/506; A61K 31/675; A61K 45/06; A61K 31/727; A61P 31/14; G01N 33/50

(52) U.S. Cl. CPC A61K 31/506; A61K 31/675; A61K 45/06; A61K 31/727; A61P 31/14; G01N 33/5091; G01N 2800/56; G01N 2800/52; G01N 2333/4716

(57) ABSTRACT

The present disclosure provides methods and compositions useful for diagnosing and treating activated alternative pathway of complement in a human subject with a SARS-CoV-2 infection. Also provided are methods of treating an infection by a betacoronavirus, e.g., SARS-CoV, MERS-CoV, or SARS-CoV-2, in a human subject. The methods of treatment include administering to the human subject a therapeutically effective amount of at least one complement factor D inhibitor. Also provided herein are compounds for treating an infection by a betacoronavirus and their use in the manufacture in a medicament for treating an infection by a betacoronavirus.

### STATEMENT REGARDING FEDERALLY SPONSORED RESEARCH

Certain embodiments of the present disclosure were made with government support under grant no. HL133113, awarded by the National Institutes of Health. The government may have certain rights in those embodiments.

### FIELD OF THE DISCLOSURE

The present disclosure relates to the fields of virology and disorders of complement activation. More specifically, the present disclosure provides methods and compositions useful for diagnosing and treating a betacoronavirus infection (e.g., a SARS-CoV-2 infection).

### BACKGROUND OF THE DISCLOSURE

The complement system acts in conjunction with other immunological systems of the body to defend against intrusion of cellular and viral pathogens. There are at least 25 complement proteins, which are found as a complex collection of plasma proteins and membrane cofactors. The plasma proteins make up about 10% of the globulins in vertebrate serum. Complement components achieve their immune defensive functions by interacting in a series of intricate but precise enzymatic cleavage and membrane binding events. The resulting complement cascade leads to the production of products with opsonic, immunoregulatory, and lytic functions. A concise summary of the biologic activities associated with complement activation is provided, for example, in The Merck Manual, 16th Edition.

While a properly functioning complement system provides a robust defense against infecting microbes, inappropriate regulation or activation of the complement pathways has been implicated in the pathogenesis of a variety of disorders, including disorders caused by infectious agents.

Non-clinical data support the role of complement 3 (C3) in mediation of lung injury elicited by infectious agents. For instance, in a mouse model of coronavirus (CoV), infection of C57BL/6J mice with mouse-adapted severe acute respiratory syndrome coronavirus (SARS-CoV) results in high-titer virus replication within the lung, induction of inflammatory cytokines and chemokines, and immune cell infiltration within the lung. See, Gralinski et al. (mBio, 2018 Oct. 9; 9(5); PMID: 30301856). Since C3 deposition was evident on day 2 and day 4 post infection with SARS-CoV, the authors hypothesize that it is likely that complement deposition contributes to pulmonary disease and inflammatory cell recruitment in the in vivo mouse model.

Studies with transgenic and/or knockout animal models further point to a role of complement system in the pathogenesis of pulmonary dysfunction following infection with viruses targeting the respiratory system. In mice treated with a mouse-infective coronavirus, infection is attenuated in C3 knockout mice, as evidenced by (a) protection against SARS-CoV-induced weight loss); (b) attenuation in pathological features (e.g., (1) presence of inflammatory cells in the large airway and parenchyma; (2) perivascular cuffing; (3) thickening of the interstitial membrane; and (4) intra-alveolar edema); (c) improved respiratory function; and/or (d) reduction in inflammatory cytokines/chemokines in the lung and its periphery. See, Gralinski et al. (supra). Gralinski further found that C3-deficient mice had reduced neutrophilia in their lungs and reduced systemic inflammation, thereby resulting in attenuation in infection. Gralinski et al. propose that inhibition of C3 complement may be therapeutically effective against coronavirus-mediated disease.

While Gralinski's studies using C3 inhibition in mice models suggest that C3 antagonism protects against SARS-CoV infection, inhibition of complement alternate pathway alone is insufficient. For instance, Factor B (fB) knockout (−/−) and complement 4 (C4) knockout (−/−) mice do not have the same protection from CoV-mediated weight loss as compared to complement 3 (C3) knockout (−/−) mice.

Excessive complement activation has also been postulated to serve an important factor that contributes to acute lung injury after Middle East respiratory syndrome coronavirus (MERS-CoV) infection. This has been demonstrated in vivo using a transgenic mouse model for MERS-CoV. See, Jiang et al. (Emerg Microbes Infect. 2018 Apr. 24; 7(1):77; PMID: 29691378). In this mouse model, MERS-CoV causes severe acute respiratory failure and high mortality accompanied by an elevated secretion of cytokines and chemokines. Histopathological analysis revealed that complement was excessively activated and concomitantly, increased concentrations of the C5a and C5b-9 complement activation products were observed in sera and lung tissues, respectively. Blocking C5aR, using an antibodies, alleviated lung and spleen tissue damage and reduced inflammatory responses. Furthermore, anti-C5aR antibody treatment attenuated viral replication in lung tissues. These results showed that blockade of the C5a-C5aR alleviates lung damage in a transgenic mice model that has been infected with MERS-CoV.

A similar finding has been reported in the context of infections mediated by influenza virus strain H5N1 (commonly called “bird flu”). See, Sun et al. (Am J Respir Cell Mo Biol. 2013 August; 49(2):221-30; PMID: 23526211). Sun showed that acute lung injury (ALI) in H5N1-infected mice was caused by excessive complement activation, as demonstrated by deposition of C3, C5b-9, and mannose-binding lectin C (MBL)-C in lung tissue, and by up-regulation of MBL-associated serine protease-2 and the complement receptors C3aR and C5aR. Treating H5N1-infected mice with a C3aR antagonist led to significantly reduced lung inflammation, alleviating ALI. Additionally, treating H5N1 challenged mice with anti-C5a antibody or depleting complement with cobra venom factor afforded protection that was similar to C3aR antagonist-treated mice. These results show a role of complement in H5N1-induced ALI and that C3aR and/or C5a antagonism may provide either direct or adjunctive options for therapy.

A recent report points to a role of nucleocapsid proteins SARS-CoV, SARS-CoV-2, and MERS-CoV in complement activation via the mannan-binding lectin (MBL) pathway (Gao et al., MedXriv, Posted Mar. 30, 2020; DOI: 10.1101/2020.03.29.20041962). It was shown that N proteins of SARS-CoV, MERS-CoV and SARS-CoV-2 bind to, and thereby potentiate MBL-associated serine protease-2 (MASP-2)-dependent complement activation, which in turn, aggravates LPS-induced pneumonia by MASP-2-involved complement activation in a mouse model. Further immunohistochemical staining of lung tissues of human COVID-19 patients showed deposit of MBL, MASP-2, C4alpha, C3 and C5b-9 in type I and type II alveolar epithelia cells, as well as inflammatory cells, some hyperplastic pneumocytes, and exudates in alveolar spaces with necrotic cell debris. Further, significantly increased serum C5a level was also observed in COVID-19 patients, particularly in severe cases. Treatment with a recombinant anti-C5a monoclonal antibody (BDB-001) conferred some clinical benefit to two patients with COVID-19.

While the above references indicate that lung injury following viral infection is mediated, in part, by the members of the complement system such as C3, C5a, C5b-9, including receptors such as C3aR and C5aR, they are silent regarding a role of complement factor D (CFD) in the pathogenesis of coronavirus-induced lung injury. Moreover, there is little information, if any, as to what therapeutic roles CFD inhibitors may play in alleviating lung injury in subjects infected with coronavirus.

There is an immediate and unmet need for developing effective strategies for the prevention, treatment and/or management of subjects, e.g., human patients, that have been infected with coronavirus.

### SUMMARY OF THE DISCLOSURE

The present disclosure is based, in part, on the finding that coronavirus spike proteins (e.g., spike protein S1 or S2 of SARS-CoV-2) bind heparan sulfate (HS) and activate the alternative complement pathway on cell surfaces (see Example 1). Treating cells with a complement factor D (CFD) inhibitor blocked the complement activation induced by S1 and/or S2 spike proteins. Using a well-established model system, the present application shows that SARS-CoV-2 spike proteins induced terminal complement C5b-9 deposition on the surface of cells via activation of the alternative pathway of complement (APC). This mechanism of APC activation and cell targeting resulted in complement-mediated killing of cells. The cytotoxic effects of the coronaviral spike proteins was blocked by complement inhibitors, specifically, by small molecule inhibitors of CFD and an anti-C5 antibody. Moreover, treatment with the CFD inhibitor also reduced serum levels of catalytic factor B (Bb) and C3c deposition on cells. Adding factor H mitigated the complement attack.

The aforementioned experimental findings supported clinical use of inhibitors of the APC, such as oral CFD inhibitors, in treating infections by betacoronaviruses, e.g., SARS-CoV, MERS-CoV, or severe acute respiratory syndrome coronavirus 2 (SARS-CoV-2). In this regard, oral CFD inhibitors such as Compound 1 and Compound 2 were selected for use in the acute clinical setting as these oral drugs allow favorable dosing for provision of sufficient and sustained trough levels of the respective compounds in vivo. These desirable pharmacological properties allows for maximization of the PK/PD potential of the drugs within their safety margins for improved and sustained APC inhibition desirable in the acute care setting.

Embodiments of the present disclosure also relate to a cellular assay for diagnosing COVID19 in patients having or suspected of having COVID19, e.g., using a rapid, easily deployable fluorescence-based method for studying complement activation and the concomitant complement-mediated destruction of GPI-AP deficient cells in the presence of spike proteins S1 and/or S2 of SARS-COV-2. Using this cellular assay, classification of COVID19 patients based on the status of the alternate pathway of complement (APC), e.g., as having activated APC or baseline APC, compared to, e.g., a normal subject. In subjects that have activated APC, the experimental evidence provided herein support the use of complement modulators, e.g., (a) FD inhibitors; (b) C5 inhibitors; (c) C3 inhibitors; (d) combination of C3 inhibitor and C5 inhibitor, (e) FH protein; or (f) FB inhibitors, in therapy of COVID19. Further disclosed herein are methods for testing for mutations in complement proteins and treating COVID19 patients with such mutations with terminal complement inhibitors. Lastly, the disclosure provides for methods of screening for test therapeutic agents that are potentially effective against cell death elicited by spike proteins, more specifically methods for screening anti-coronaviral drugs (esp. anti-SARS-CoV-2 agents) using aforementioned rapid, easily deployable, cellular assay.

The present disclosure provides methods of treating a complement mediated disorder caused by a virus, e.g., a betacoronavirus such as SARS-CoV, MERS-CoV, and SARS-CoV-2, in a human subject.

In one aspect, the present disclosure features a method of treating a betacoronavirus infection in a human subject, which includes administering a therapeutically effective amount of a complement factor D (CFD) inhibitor, e.g., Compound 1, Compound 2, Compound 3, or Compound 4:

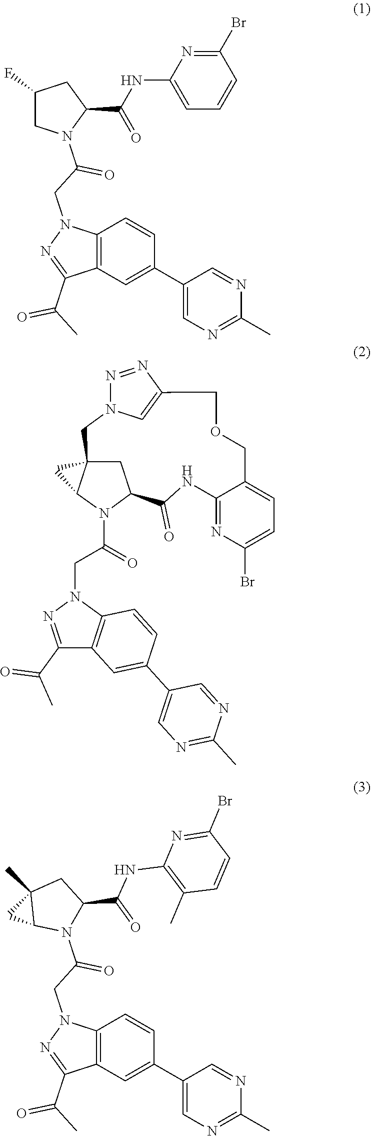

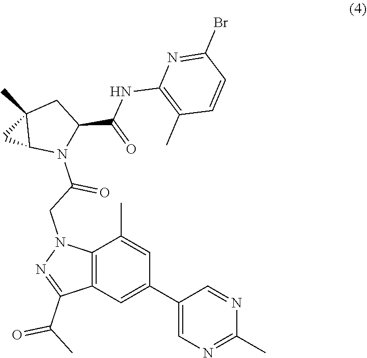

or a pharmaceutically acceptable salt thereof. Exemplary CFD inhibitors are described in e.g., U.S. Pat. Nos. 9,598,446; 9,643,986; 9,663,543; 9,695,205; 9,732,103; 9,732,104; 9,758,537; 9,796,741; 9,828,396; 10,000,516; 10,011,612; 10,005,802; 10,081,645; 10,087,203; 10,092,584; 10,100,072; 10,106,563; 10,138,225; 10,189,869; 10,253,053; 10,287,301; 10,301,336; 10,370,394; 10,385,097; 10,428,095; 10,454,956; 10,550,140; 10,660,876; 10,662,175; 10,689,409; 10,807,952; 10,822,352; 10,906,887; 10,919,884, 11,084,800; International Publication Nos. WO 2020/041301, WO 2020/041301, and WO 2020/051532; and U.S. Patent Publication Nos. 2019-0382376, 2020-0062790, and 2020-0262818, the contents of which are incorporated herein by reference in their entirety.

In some embodiments, the betacoronavirus is at least one betacoronavirus selected from SARs-CoV, MERS-CoV, and SARS-CoV-2. In some embodiments, the betacoronavirus is SARS-CoV-2. For example, the subject has a laboratory confirmed SARS-CoV-2 infection (e.g., COVID-19) as determined by polymerase chain reaction (PCR), e.g., PCR positive in a sample collected <72 hours prior to treatment or z 72 hours by <14 days prior to treatment and non-improving or progressive disease suggestive of an ongoing SARS-CoV-2 infection (e.g., COVID-19).

In some embodiments, the human subject is exhibiting one or more respiratory symptoms, e.g., at least one of inflammation of cells in the large airway and parenchyma, perivascular cuffing, thickening of the interstitial membrane, intra-alveolar edema, rhinorrhea, sneezing, sore throat, pneumonia, lung ground-glass opacity, RNAemia, and acute respiratory distress syndrome (ARDS).

In some embodiments, the human subject is exhibiting one or more systemic disorders, e.g., at least one of acute cardiac injury, cough, cytokine storm, nausea, vomiting, diarrhea, dyspnea, anosmia (loss of smell or taste), fatigue, fever, headache, muscle or body ache, hemoptysis, hypoxemia, lymphopenia, renal injury (e.g., acute kidney injury; AKI), stroke, septic shock, sputum production, nasal congestion, refractory thrombosis, antiphospholipid syndrome, seizure, renal failure, and myocardial infarction. In some embodiments, the systemic disorder is associated with thrombotic microangiopathy (TMA).

In some embodiments, the human subject is suffering from critical viral disease comprising shortness of breath (e.g., resting rate >30 breaths/minute; oxygen saturation <93% at rest or arterial oxygen partial pressure (PaO2)/fraction of inspired oxygen (FiO2) <300 mmHg (1 mmHg=0.133 kPa)). In some embodiments, the human subject is suffering from critical viral disease comprising respiratory failure requiring mechanical ventilation; respiratory shock; severe pneumonia; acute lung injury (ALI); ARDS requiring oxygen supplementation; and/or combined failure of non-respiratory organs (e.g., heart, kidney) that require ICU monitoring. In some embodiments, the human subject is suffering from critical viral disease displays at least one symptom selected from (a) progressive reduction of peripheral blood lymphocytes; (b) progressive increase of peripheral inflammatory cytokines such as IL-6 and C-reactive protein; (c) progressive increase of lactate; and (d) rapid progression of one or more lung pathologies.

In some embodiments, the method reduces activation of terminal complement (e.g., C5b9) deposition in alveolar cells, in the human subject. In some embodiments, the method reduces serum levels of catalytic factor B (Bb) in the human subject. In some embodiments, the method reduces alveolar cell death in the human subject.

In some embodiments, the method reduces the risk or duration of hospitalization of the human subject.

In some embodiments, the method reduces the risk or duration of need for respiratory intubation of the human subject.

In some embodiments, the method reduces the risk of developing one or more respiratory syndromes, e.g., at least one of inflammation of cells in the large airway and parenchyma, perivascular cuffing, thickening of the interstitial membrane, intra-alveolar edema, rhinorrhea, sneezing, sore throat, pneumonia, lung ground-glass opacity, RNAemia, and acute respiratory distress syndrome (ARDS), in the human subject.

In some embodiments, the method reduces the risk of developing one or more systemic disorders, e.g., at least one of acute cardiac injury, cough, cytokine storm, nausea, vomiting, diarrhea, dyspnea, anosmia (loss of smell or taste), fatigue, fever, headache, muscle or body ache, hemoptysis, hypoxemia, lymphopenia, renal injury (e.g., acute kidney injury; AKI), stroke, septic shock, sputum production, nasal congestion, refractory thrombosis, antiphospholipid syndrome, seizure, renal failure, and myocardial infarction, in the human subject. In some embodiments, the systemic disorder is renal failure. In some embodiments, the method reduces the risk of developing one or more systemic disorders associated with thrombotic microangiopathy (TMA) elicited by betacoronaviral infection.

In some embodiments, the method reduces the risk of developing critical viral disease is reduced.

In some embodiments, the critical viral disease comprises severe shortness of breath. In some embodiments, the critical viral disease comprises at least one of acute lung injury (ALI), AKI, ARDS, failure of non-respiratory organs, respiratory failure, respiratory shock, stroke, and severe pneumonia.

In some embodiments, the method reduces the risk of death of the human subject.

In some embodiments, the method reduces the risk or duration of need for non-invasive mechanical ventilation in the human subject.

In some embodiments, the non-invasive mechanical ventilation is supplemental oxygen. In embodiments where the human subject is on chronic supplemental oxygen, the supplemental oxygen pertains to an increase refers to an increase oxygen requirement above baseline. In some embodiments, the non-invasive mechanical ventilation is CPAP. In some embodiments, the non-invasive mechanical ventilation is BiPAP.

In some embodiments, the method reduces the risk or duration of need for extracorporeal membrane oxygenation in the human subject.

In some embodiments, the method reduces the risk or duration of need for invasive mechanical ventilation (e.g., high-flow oxygen therapy) in the subject.

In some embodiments, the at least one factor D inhibitor is administered orally. In some embodiments, the at least one factor D inhibitor is provided in a solid-based dosage form (e.g., a powder-filled capsule dosage form or a tablet dosage form). In some embodiments, the at least one factor D inhibitor is provided in a liquid-filled capsule dosage form. In some embodiments, the at least one factor D inhibitor is provided in a gel-based dosage form.

In some embodiments, the at least one factor D inhibitor is administered via a nasogastric tube, e.g., as a solution or suspension. The solution or suspension may be formed by providing the at least one factor D inhibitor in the form of particles and adding water to the particles to form the solution or suspension.

In some embodiments, said administering comprises administering to the human subject a loading dose of the at least one CFD inhibitor followed by a maintenance dose of the at least one factor D inhibitor.

In some embodiments, the maintenance dose comprises about 600 mg to about 1200 mg (e.g., about 800 mg to about 1000 mg) of the at least one CFD inhibitor administered in one or more doses per day. For example, the maintenance dose may be administered in four doses per day (QID), in which each does may comprise, e.g., about 150 mg to about 300 mg or about 200 mg to about 250 mg of the at least one CFD inhibitor. In some embodiments, the maintenance dose may be administered in three doses per day (TID), in which each does may comprise, e.g., about 100 mg to about 300 mg or about 150 mg to about 200 mg of the at least one CFD inhibitor. In some embodiments, the maintenance dose may be administered in four doses per day (QID), in which each does may comprise, e.g., about 75 mg to about 200 mg or about 100 mg to about 150 mg.

In some embodiments, the loading dose comprises about 200 mg to about 500 mg (e.g., about 250 mg to about 450 mg, about 300 mg to about 400 mg, about 200 mg, about 250 mg, about 300 mg, about 350 mg, about 400 mg, about 450 mg, or about 500 mg) of the at least one CFD inhibitor.

In some embodiments, the human subject is less than 70 years of age (e.g., less than 50 years of age, less than 25 years of age, or less than 18 years of age). In certain embodiments in which the human is less than 70 years of age (e.g., less than 50 years of age, less than 25 years of age, or less than 18 years of age), said administering comprises administering to the human subject a loading dose of about 400 mg of the at least one CFD inhibitor followed by a maintaining dose of the at least one CFD inhibitor, wherein the maintenance dose comprises about 250 mg of the at least one CFD inhibitor administered four times a day.

In some embodiments, the human subject is at least 70 years of age. In certain embodiments in which the human is at least 70 years of age, said administering comprises administering to the human subject a loading dose of about 300 mg of the at least one CFD inhibitor followed by a maintaining dose of the at least one CFD inhibitor, wherein the maintenance dose comprises about 200 mg of the at least one CFD inhibitor administered four times a day.

In some embodiments, the method provides a minimum mean plasma concentration (C_{trough}) of from about 100 ng/mL to about 600 ng/mL (e.g., from about 150 ng/mL to about 300 ng/mL) of the at least one factor D inhibitor. In some embodiments, the method provides a C_{trough} of at least about 100 ng/mL (e.g., at least about 150 ng/mL, at least about 235 ng/mL, at least about 300 ng/mL, or at least about 600 ng/mL) of the at least one CFD inhibitor.

In some embodiments, the method provides a maximum plasma concentration (Cₘₐₓ) of the at least one CFD inhibitor of less than about 1000 ng/mL (e.g., less than about 500 ng/mL or less than about 300 ng/mL).

In some embodiments, the method further includes determining Bb level elevation in the human subject prior to administering the at least one CFD inhibitor.

In some embodiments, the human subject has at least one pre-existing condition that increases their risk of one or more of pneumonia, acute respiratory distress syndrome, respiratory failure, septic shock, organ failure, cytokine storm, or death. The pre-existing condition may include at least one condition selected from cardiovascular disease, chronic respiratory disease, diabetes, hypertension, immune deficiency, and obesity.

In some embodiments, the at least one CFD inhibitor is administered in combination with a second therapeutic agent, e.g., remdesivir or a glycosaminoglycan. In some embodiments, the at least one CFD inhibitor is administered in combination with remdesivir, which may be administered as a 200-mg intravenous loading dose, followed by a 100-mg once-daily intravenous maintenance dose. In some embodiments, the at least one CFD inhibitor is administered in combination with a glycosaminoglycan, e.g., heparan sulfate, an α2,3- and α2,6-sialylated N-glycan, or an analog thereof. In some embodiments, the glycosaminoglycan is a ReGeneraTing Agent or PI-88.

In some embodiments, the human subject does not have an alanine aminotransferase (ALT) or aspartate aminotransferase (AST) level >5 times the upper limit of normal.

In some embodiments, the human subject does not have an estimated glomerular filtration rate (eGFR) <30 mL/min (e.g. <20 mL/min).

In some embodiments, the human subject has not received five or more doses of remdesivir prior to the start of treatment.

In some embodiments, the human subject has not received two or more doses of >60 mg prednisone or an equivalent thereof in the 7 days prior to the start of treatment.

In some embodiments, the human subject has not received a small molecule tyrosine kinase inhibitor, such as a Janus kinase (JAK) inhibitor (e.g., baricitinib, ibrutinib, acalabrutinib, imatinib, or gefitinib) in the 4 weeks prior to the start of treatment.

In some embodiments, the human subject has not received a monoclonal antibody targeting a cytokine, such as a tumor necrosis factor (TNF) inhibitor, anti-IL-1 (e.g., anakinra or canakinumab), or anti-IL-6 (e.g., tocilizumab, sarilumab, or sitlukimab) in the 4 weeks prior to the start of treatment.

In some embodiments, the human subject has not received a monoclonal antibody targeting T-cells (e.g., abatacept) in the 4 weeks prior to the start of treatment.

In some embodiments, the human subject has not received a monoclonal antibody tarting B-cells (e.g., rituximab, or one that targets multiple cell lines including B-cells) in the 3 months prior to the start of treatment.

In some embodiments, the human subject has not received a granulocyte-macrophage colony-stimulating factor (GM-CSF) agent (e.g., sagramostim) within 2 months prior to the start of treatment.

In some embodiments, the subject has not received an immunosuppressant in the 4 weeks prior to the start of treatment.

In some embodiments, the human subject has not received a live vaccine prior to the start of treatment.

In some embodiments, the human subject does not have active tuberculosis.

In some embodiments, the human subject does not have a known history of a human immunodeficiency virus (HIV) infection.

In some embodiments, the human subject does not have a known history of a hepatitis B virus (HBV) infection.

In some embodiments, the human subject does not have a known history of a hepatitis C virus (HCV) infection.

In some embodiments, the human subject does not have a known history of pulmonary alveolar proteinosis (PAP).

In some embodiments, the human subject does not have an active malignancy.

In some embodiments, the human subject does not have immunodeficiency.

In some embodiments, the human subject does not have an uncontrolled opportunistic infection.

In some embodiments, the human subject does not have uncontrolled cirrhosis.

In some embodiments, the human subject is not tested positive for an influenza virus.

In some embodiments, the human subject is prohibited from receiving a small molecule tyrosine kinase inhibitor, such as a JAK inhibitor (e.g., baricitinib, ibrutinib, acalabrutinib, imatinib, or gefitinib).

In some embodiments, the human subject is prohibited from receiving a monoclonal antibody targeting a cytokine, such as a TNF inhibitor, anti-IL-1 (e.g., anakinra or canakinumab), or anti-IL-6 (e.g., tocilizumab, sarilumab, or sitlukimab).

In some embodiments, the human subject is prohibited from receiving a monoclonal antibody targeting T-cells (e.g., abatacept).

In some embodiments, the human subject is prohibited from receiving a monoclonal antibody targeting B-cells (e.g., rituximab or a monoclonal antibody targeting multiple cell lines including B-cells).

In some embodiments, the human subject is prohibited from receiving a GM-CSF agent (e.g., sagramostim).

In some embodiments, the human subject is prohibited from receiving an immunosuppressant.

In some embodiments, the human subject is prohibited from receiving chloroquine or hydroxychloroquine.

In some embodiments, the human subject has activated pathway of complement (APC).

In another aspect, the present disclosure features a CFD inhibitor, e.g., any one of Compounds 1-4, or a pharmaceutically acceptable salt thereof, for use in a method (e.g., any one of the methods of treatment described herein) of treating a betacoronavirus infection or a disease caused by a betacoronavirus, e.g., SARS, MERS, or COVID-19.

In another aspect, the present disclosure features the use of a CFD inhibitor, e.g., any one of Compounds 1-4, or a pharmaceutically acceptable salt thereof in the manufacture of a medicament for use in a method (e.g., any one of the methods of treatment described herein) of treating a betacoronavirus infection or a disease caused by a betacoronavirus, e.g., SARS, MERS, or COVID-19.

In another aspect, the present disclosure provides a method for diagnosing a SARS-CoV-2 infection (e.g., COVID-19) in a patient having or suspected of having a SARS-CoV-2 infection (e.g., COVID-19), comprising (a) incubating serum obtained from the patient and, optionally, a control serum with a plurality of glycosylphosphatidylinositol-anchored protein (GPI-AP) deficient cells; (b) measuring cell viability of GPI-AP deficient cells in the incubate of (a); (c) comparing the cell viability of the GPI-AP deficient cells incubated with the patient's serum with (1) a reference standard or (2) the viability of GPI-AP deficient cells incubated with the control serum, wherein a reduction in the cell viability in the patient serum incubate compared to the reference standard or the control serum incubate is indicative that the patient has or is likely to have a SARS-CoV-2 infection (e.g., COVID-19).

In another aspect, the present disclosure provides a method for diagnosing activated APC in a patient with a SARS-CoV-2 infection, including (a) incubating serum obtained from the patient and, optionally, a control serum with a plurality of GPI-AP deficient cells; (b) measuring cell viability of GPI-AP deficient cells in the incubate of (a); (c) comparing the cell viability of GPI-AP deficient cells incubated with the patient's serum with (1) a reference standard or (2) the viability of GPI-AP deficient cells incubated with the control serum, wherein a reduction in the cell viability in the patient serum incubate compared to the reference standard or the control serum incubate is indicative that the patient with a SARS-CoV-2 infection has activated APC.

In another aspect, the present disclosure provides a method of classifying a patient with a SARS-CoV-2 infection (e.g., COVID-19) based on activated APC status, which includes the steps of: (a) diagnosing APC in a patient with a SARS-CoV-2 infection according to the method of claim 94; and (b) classifying the patient as having activated APC (APC+ infection) if a reduction in the cell viability of the GPI-AP deficient cells in the patient serum incubate compared the cell viability of the GPI-AP deficient cells in the reference standard or the control serum incubate is observed; or classifying the patient as having infection with baseline APC if the cell viability of the GPI-AP cells in the patient serum incubate is unchanged or elevated compared to the cell viability of the GPI-AP cells in the reference standard or the control serum incubate.

In another aspect, the present disclosure provides a method for treating a SARS-CoV-2 infection (e.g., COVID-19), which includes classifying a patient according to the any method of classifying a patient with a SARS-CoV-2 infection (e.g., COVID-19) disclosed herein, if the patient is classified as having activated APC, administering to the patient a therapeutically effective amount of any of the CFD inhibitor for use disclosed herein.

In some embodiments of any of the preceding aspects, the patient has COVID-19.

In some embodiments of any of the preceding aspects, the method comprises incubating a control serum with a plurality of GPI-AP deficient cells. The control serum may be a serum obtained from a healthy subject, such as a subject who has never had a SARS-CoV-2 infection or one who has fully recovered from a SARS-CoV-2 infection (e.g., COVID-19).

In some embodiments of any of the preceding aspects, the control serum is supplemented with SARS-CoV-2 N protein and/or a spike protein from a benign human coronavirus (e.g., HCoV-OC43).

In some embodiments of any of the preceding aspects, the cell viability of the GPI-AP deficient cells incubated with the patient's serum is compared with a reference standard, wherein the reference standard is a baseline cell viability (e.g., at least 70%, at least 80%, at least 90%, at least 95%, or at least 99%) of GPI-AP deficient cells in healthy human serum. Cell viability may be measured using a fluorescence assay, such as fluorescence activated cell sorter (FACS).

In some embodiments of any of the preceding aspects, further including (3) measuring deposition of a complement protein on the surface of the GPI-AP deficient cells, wherein the complement protein is selected from the group of C5b9, C4d, C3c, and any combination thereof. The deposition of the complement protein may be measured using a fluorescence assay, such as FACS.

In some embodiments of any of the preceding aspects, the GPI-AP deficient cells are PIG null blood cells, such as PIGA null lymphoblasts TF1 ((TF1PIGAnull) cells.

In another aspect, the present disclosure provides a method of assessing a risk of a patient with a SARS-CoV-2 infection (e.g., COVID-19) to develop a severe vascular complication (e.g., disseminated thrombosis and/or multi-organ failure), including (a) detecting the presence or absence of one or more mutations in complement proteins in a cell sample obtained from the patient; and (b) identifying the patient as being at high risk for developing the vascular complication (e.g., disseminated thrombosis and/or multi-organ failure) if the patient's cell sample comprises one or more mutations in complement proteins.

In some embodiments, the one or more mutations in complement proteins comprises a gain of function (GOF) mutation in a complement activating factor (e.g., complement C3 or complement factor B) and/or a loss of function (LOF) mutation in a complement inhibitory factor (e.g., one or more of complement factor H (CFH); a CFH-related protein selected from complement factor H receptor 1 (CFHR1), complement factor H receptor 2 (CFHR2), complement factor H receptor 3 (CFHR3), complement factor H receptor 1 (CFHR4), and complement factor H receptor 5 (CFHR5); complement factor I (CFI); membrane cofactor protein (MCP or CD46); thrombomodulin (THBD); and complement receptor 1 (CR1).

In another aspect, the present disclosure provides a method for treating a patient with a SARS-CoV-2 infection (e.g., COVID-19) who is at risk for developing a severe vascular complication (e.g., disseminated thrombosis and/or multi-organ failure), including (a) identifying a patient as being at high risk for developing the severe vascular complication according to any one of the methods of assessing a risk of a patient with SARS-CoV-2 infection (e.g., COVID-19) to develop a severe vascular complication (e.g., disseminated thrombosis and/or multi-organ failure) disclosed herein; and (b) administering the patient a therapeutically effective amount of any one of the CFD inhibitor for use disclosed herein.

In another aspect, the present disclosure provides method of screening for test agents that inhibit SARS-CoV-2 spike protein-mediated cell death, including (a) incubating GPI-AP deficient cells with human serum comprising recombinant SARS-CoV-2 spike proteins S1 and/or S2 for a period sufficient to induce APC on the GPI-AP cells to form an incubate; (b) treating the incubate (i) with a test agent to produce a test incubate and (ii) without a test agent (e.g., vehicle only) to produce a null incubate; (c) measuring cell viability of the GPI-AP deficient cells in the test and null incubates; and (d) comparing the cell viability of the GPI-AP deficient cells in the test incubate to the cell viability in the null incubate; in which an increase in the cell viability in the test incubate compared to the cell viability in the null incubate indicates that the test agent is capable of inhibiting SARS-CoV-2 mediated cell death. Cell viability may be measured using a fluorescence assay, such as FACS.

In some embodiments, the one or more control agent comprises an agent which effectively blocks the binding of SARS-CoV-2 spike proteins to cell surface (e.g., heparan sulfate or a derivative thereof).

In some embodiments, the method further includes determining deposition of complement proteins C5b9, C4d, and/or C3c on the surface of GPI-AP deficient cells in the null sample and the test sample. Deposition of complement proteins may be measured using a fluorescence assay, such as FACS.

In some embodiments, step (b) further includes (iii) treating the incubate with one or more control agents to produce one or more control incubates, and wherein the method further comprises (e) comparing the cell viability of GPI-AP deficient cells in the test incubate to the cell viability of the GPI-AP deficient cells in the one or more control incubates, wherein an increase in the cell viability in the test incubate compared to the cell viability in the one or more control incubates indicates that the test agent is capable of inhibiting SARS-CoV-2 spike protein-mediated cell death. The cell viability of GPI-AP cells in the one or more control incubates may be higher than that of GPI-AP cells in the null incubate. Cell viability may be measured using a fluorescence assay, such as FACS. The method may further include determining deposition of complement proteins C5b9, C4d, and/or C3c on the surface of GPI-AP deficient cells in the one or more control samples, which may be measured using a fluorescence assay, such as FACS.

In some embodiments, GPI-AP deficient cells are PIGA null blood cells (e.g., TF1PIGAnull cells).

In another aspect, the present disclosure provides methods for testing a patient for mutations in complement related genes, specifically, for mutations in genes that inhibit regulation of APC or mutations that directly activate APC. Patients can be tested for mutations in complement factor H (CFH), CFH-related proteins (CFHR1, CFHR2, CFHR3, CFHR4, CFHR5), complement factor I (CFI), CD46 (membrane cofactor protein, MCP), complement factor B (CFB), complement component C3 (C3), thrombomodulin (THBD), plasminogen, diacylglycerolkinase-E (DGKE), complement factor D (CFD), and complement receptor 1 (CR1).

In some embodiments, patients are tested for loss of function mutation(s) in a complement inhibitory factor (CFH, CFI, CD46 (MCP), THBD, CR1) or a gain of function mutation(s) of a complement activating factor (CFB, C3). Such mutations are known in the art. These patients are likely to be predisposed to uncontrolled complement activation, which could lead to disseminated thrombosis and multi-organ failure in the setting of a complement amplifying trigger such as infection (COVID-19), as well as surgery, pregnancy, or autoimmune disease.

In some embodiments, depending on the mutation(s), a COVID-19 patient may benefit from a terminal complement inhibitor (e.g., anti-C5 antibody (eculizumab)) or an APC inhibitor such as a CFD (e.g., Compound 1; “danicopan”) or a Factor B inhibitor (IONIS-FB-LRx), as well as administration of Factor H.

In one embodiment, the method includes the steps of (a) incubating serum obtained from a patient having or suspected of having COVID-19 with a plurality of GPI-AP deficient cells; and (b) performing a cell viability assay on the cells from step (a). In another embodiment, the method further comprises the step of diagnosing the patient as having an activated alternative pathway of complement (APC) based on a statistically significant increased difference of non-viable cells from the patient's serum as compared to a control, e.g., a healthy subject.

In another embodiment, a method for diagnosing activated APC in a COVID-19 patient includes the steps of (a) incubating serum obtained from a patient having COVID-19 with a plurality of GPI-AP deficient cells; (b) performing a cell viability assay on the cells from step (a); and (c) diagnosing the patient as having activated APC based on a statistically significant increased difference of non-viable cells from the patient's serum as compared to a control, e.g., a healthy subject.

In another aspect, the present disclosure provides a method for treating a SARS-CoV-2 infection (e.g., COVID-19) in a subject, the method including the steps of administering an effective amount of a C5 inhibitor and a C3 inhibitor to a patient with a SARS-CoV-2 infection diagnosed with activated APC according to a method described herein.

In another aspect, the present disclosure provides a method for treating activated APC in a COVID-19 patient including the step of administering an effective amount of a C5 inhibitor and a C3 inhibitor to a patient diagnosed with activated APC based on the performance of a cell viability assay on a plurality of GPI-AP deficient cells that have been incubated with serum obtained from the patient, wherein the diagnosis is based on a statistically significant increased difference of non-viable cells from the patient's serum as compared to a control, e.g., a healthy subject.

In another aspect, the present disclosure provides a method for treating activated APC in a COVID-19 patient including the step of administering an effective amount of a CFD inhibitor to a patient diagnosed with activated APC based on the performance of a cell viability assay on a plurality of GPI-AP deficient cells that have been incubated with serum obtained from the patient, wherein the diagnosis is based on a statistically significant increased difference of non-viable cells from the patient's serum as compared to a control, e.g., a healthy subject.

In another aspect, the present disclosure provides a method for treating activated APC in a COVID-19 patient including the step of administering an effective amount of a CFD inhibitor to a patient diagnosed with activated APC based on the performance of a cell viability assay on a plurality of GPI-AP deficient cells that have been incubated with serum obtained from the patient, wherein the diagnosis is based on a statistically significant increased difference of non-viable cells from the patient's serum as compared to a control, e.g., a healthy subject.

The present disclosure also provides a method for treating activated APC in a COVID-19 patient including the steps of (a) incubating serum obtained from a COVID-19 patient with a plurality of GPI-AP deficient cells; (b) performing a cell viability assay on the cells from step (a); (c) diagnosing the patient as having activated APC based on a statistically significant increased difference of non-viable cells from the patient's serum as compared to a control, e.g., a healthy subject; and (d) administering an effective amount of a Factor D inhibitor to the patient.

In an alternative embodiment, a method for treating activated APC in a COVID-19 patient includes the steps of (a) incubating serum obtained from a COVID-19 patient with a plurality of GPI-AP deficient cells; (b) performing a cell viability assay on the cells from step (a); (c) diagnosing the patient as having activated APC based on a statistically significant increased difference of non-viable cells from the patient's serum as compared to a control, e.g., a healthy subject; and (d) administering an effective amount of a C5 inhibitor and a C3 inhibitor to the patient. In particular embodiments, the plurality of GPI-AP deficient cells is biochemically treated to remove GPI-AP. In a specific embodiment, the plurality of GPI-AP deficient cells is a PIGA null mutant cell line. In certain embodiments, the cell viability assay is the WST-1 cell viability assay. In some embodiments, the C5 inhibitor is eculizumab, ravulizumab, coversin, cemdisiran, LFG-316, SOB1005, SKY59, REGN3918, TNX-558, neutrazumab, CCX188, ABP959, GNR-45, zimura, RA101495, ISU305, mubodina, IFX-1, ALS-205, DF2593A, or IPH5401.

In some embodiments, the C3 inhibitor is AMY-101, APL-1, APL-2, or APL-9. In other embodiments, the patient is further treated with Factor H.

In some embodiments, the CFD inhibitor is:

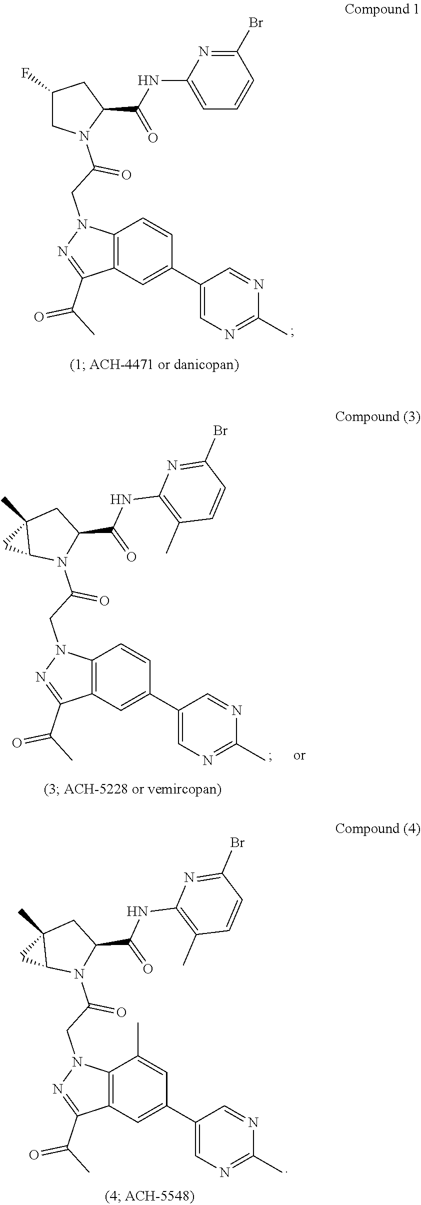

The present disclosure also provides a method for treating activated APC in a COVID-19 patient including the steps of administering an effective amount of Factor H to a COVID-19 patient diagnosed with activated APC according to the method described herein. In another embodiment, a method for treating activated APC in a COVID-19 patient comprises the step of administering an effective amount of Factor H to a patient diagnosed with activated APC based on the performance of a cell viability assay on a plurality of GPI-AP deficient cells that have been incubated with serum obtained from the patient, wherein the diagnosis is based on a statistically significant increased difference of non-viable cells from the patient's serum as compared to a control, e.g., a healthy subject.

In some embodiments, the method for treating activated APC in a COVID-19 patient includes the steps of (a) incubating serum obtained from a COVID-19 patient with a plurality of GPI-AP deficient cells; (b) performing a cell viability assay on the cells from step (a); (c) diagnosing the patient as having activated APC based on a statistically significant increased difference of non-viable cells from the patient's serum as compared to a control, e.g., a healthy subject; and (d) administering an effective amount of Factor H inhibitor to the patient. In some embodiments, the patient is further treated with either a CFD inhibitor or a C5 inhibitor and a C3 inhibitor.

In some embodiments, the COVID-19 patient is tested for mutations in a complement-related gene. In one embodiment, the mutation comprises a loss of function mutation in a complement inhibitory factor or a gain of function mutation of a complement activating factor. In a specific embodiment, the complement inhibitory factor comprises complement factor H (CFH), complement factor I (CFI), CD46, thrombomodulin (THBD), and complement receptor 1 (CR1). In another specific embodiment, the complement activating factor comprises complement factor B (CFB) and complement component C3.

Numerous other aspects are provided in accordance with these and other aspects of the disclosure. Other features and aspects of the present disclosure will become more fully apparent from the following detailed description and the appended claims.

### BRIEF DESCRIPTION OF THE DRAWINGS

FIGS. 1A-1F are graphs showing cell death (%) of TF1PIGAnull cells treated with 20% NHS and preincubated with diluted (2.5 μg/mL to 20 μg/mL). (FIG. 1A) SARS-CoV-2 spike protein subunit 1 (S1), (FIG. 1B) SARS-CoV-2 spike protein subunit 2 (S2), (FIG. 1C) N proteins (N), (FIG. 1D) HCoV-OC43 S proteins, (FIG. 1E) S1 and Compound 2 (1 μM), and (FIG. 1F) S1 and Compound 2 (1 μM). The dotted line at 20% non-viable cells was established as a threshold for a positive mHam based on a receiver operative curve. All experiments were repeated for at least three times. Statistical significance was calculated between each coronavirus protein treated group and the NHS treated group (*: P<0.05, **: P<0.01, ***: P<0.001, ****: P<0.0001, NS: not significant). NHS indicates normal human serum, NHS(H) indicates heat-inactivated normal human serum, and FD Inh indicates Compound 2.

FIGS. 2A and 2B are graphs showing C5b-9 deposition on TF1PIGAnull cells after adding NHS preincubated with S1, S2, N, and HCoV-OC43 S proteins (2.5 μg/mL to 20 μg/mL) in (FIG. 2A) all pathway buffer (GVB⁺⁺ pH 7.4) and (FIG. 2B) APC-specific buffer (CVB0 10 mM Mg-EGTA pH 6.4) as determined by flow cytometry. All experiments were repeated for six times. Statistical significance was calculated between each 20 μg/mL coronavirus protein treated group and the NHS treated group (*: P<0.05, **: P<0.01, ***: P<0.001, ****: P<0.0001, NS: not significant). NHS indicates normal human serum.

FIGS. 3A and 3B are graphs showing the reduction in the complement activity of SARS-Cov-2 spike proteins after heat inactivation. Heating SARS-CoV-2 spike protein subunit 1 (S1) and subunit 2 (S2) for 30 min at 100° C. before exposure to TF1PIGAnull cells markedly reduced the C5b-9 accumulation induced by S1 (FIG. 3A) and S2 (FIG. 3B) in GVB++ buffer. Heat-inactivation of S1 inhibited C5b-9 deposition in a dose-dependent manner, and S2-induced C5b-9 was decreased to the baseline NHS level with heat denaturing. NHS indicates normal human serum.

FIGS. 4A-4D are graphs showing the blocking of (FIG. 4A) S1-induced (20 μg/mL S1) C5b-9 deposition by Compound 2 and eculizumab; (FIG. 4B) S1-induced (20 μg/mL S1) C3c deposition by Compound 2, but not eculizumab; (FIG. 4C) S2-induced (20 μg/mL S2) C5b-9 deposition by Compound 2 and eculizumab; and (FIG. 4D) S2-induced (20 μg/mL S2) C3c deposition by Compound 2, but not eculizumab, as determined by flow cytometry. Unpaired Student t-test P-values indicate statistical significance (*: P<0.05, *: P<0.01, NS: not significant). NHS indicates normal human serum, FD Inh indicates Compound 2, and Anti-C5Ab indicates eculizumab.

FIG. 4E shows a representative flow cytometry analysis demonstrated 1 μM factor D inhibitor completely blocked the C5b-9 (left panel) and C3c deposition (right panel) triggered by 20 μg/mL S1 proteins. C5 inhibition with 50 μg anti-C5 antibody prevented S1-induced C5b-9 deposition, but not C3c accumulation. NHS indicates normal human serum, FD Inh indicates factor D inhibitor (Compound 2), Anti-C5Ab indicates eculizumab.

FIGS. 5A and 5B are graphs showing the effects of SARS-CoV-2 spike proteins on C4d position on cell surface. (FIG. 6A) SARS-CoV-2 spike protein subunit 1 (S1) triggered slight increase in C4d deposition on TF1PIGAnull cells with a dose dependent trend. (FIG. 5B) Incubation of NHS with SARS-CoV-2 spike protein subunit 2 (S2) did not markedly elevate C4d deposition from the baseline NHS level. NHS indicates normal human serum.

FIG. 6A is a graph showing serum Bb concentration measured by ELISA after incubation of S1 and S2 with NHS in the presence and absence of TF1PIGAnull cells in APC-specific buffer. FIG. 6B is a graph showing serum Bb concentration as measured by ELISA after incubation of S1 and S2 with NHS in the presence TF1PIGAnull cells in APC-specific buffer, with and without the addition of Compound 2 (1 μM) or eculizumab (50 μg). All experiments were repeated for three times. Unpaired Student t-test P-values indicate statistical significance (*: P<0.05, *: P<0.01, NS: not significant). NHS indicates normal human serum, FD Inh indicates Compound 2, and anti-C5Ab denotes eculizumab.

FIG. 7 is a representative flow cytometry graph showed the increased binding of 20 μg/mL His-tagged SARS-CoV-2 S1 subunit and S2 subunit to TF1PIGAnull cells. Minimal binding of SARS-CoV-2 N protein and HCoV-OC43 S protein was detected on TF1PIGAnull cells.

FIGS. 8A-8D are graphs showing that SARS-CoV-2 spike proteins bind to heparan sulfate and interfere with factor H function. Co-incubating 5 μg/mL His-tagged SARS-CoV-2 spike proteins with 2 mg/mL heparan sulfate solution completely blocked the binding of S1 subunit (FIG. 8A) and S2 subunit (FIG. 8B) to TF1PIGAnull cells. Supplementing NHS with 10 μg purified factor H protein significantly inhibited C5b-9 (FIG. 8C) and C3c deposition (FIG. 8D) triggered by 20 μg/mL S1 in AP-specific buffer. NHS indicates normal human serum, NaHS indicates heparan sulfate solution, FH indicates purified factor H protein.

FIG. 9 is a flow cytometry graph showing that the binding of binding of 20 μg/mL His-tagged S1 was not significantly reduced on TF1PIGAnull cells treated with 50 U/mL α2-3,6,8 sialidase. Sialidase indicates α2-3,6,8 sialidase.

FIG. 10 shows a proposed model for COVID-19. Under normal condition, factor H binds to heparan sulfate on the cell surface and interact with C3b, which facilitates factor I cleavage and deactivation of C3b. Upon infection, SARS-CoV-2 spike protein binds to heparan sulfate on the cell surface and interfere with factor H function, which facilitates factor B binding to C3b and cleavage by factor D. Abbreviation: CFH, factor H; AT III, antithrombin III; FGF2, fibroblast growth factor 2; VEGF, vascular endothelial growth factor; TGF-β, transforming growth factor β; HB-EGF, heparin-binding epidermal growth factor; ecSOD, extracellular superoxide dismutase.

FIG. 11 shows graphs depicting the simulated serum ALXN2040 (Compound 1; danicopan) concentrations on Days 1 and 4 using a 400 mg loading dose and 250 mg QID maintenance dose for individuals <50 years and 50-69 years and a 300 mg loading dose and 200 mg QID maintenance dose for individuals ≥70 years. The solid lines indicate the median values. The shaded regions indicate 95% prediction interval. The dotted line indicates the IC₅₀ value (235 ng/mL).

### DETAILED DESCRIPTION

As used herein, the word “a” or “plurality” before a noun represents one or more of the particular nouns. For example, the phrase “a mammalian cell” represents “one or more mammalian cells.”

The singular forms “a,” “an,” and “the” include plural reference unless the context clearly dictates otherwise.

The term “about”, particularly in reference to a given quantity or number, is meant to encompass deviations within plus or minus ten percent (±10%), (e.g., ±5%).

The term “pharmaceutical formulation” refers to preparations which are in such form as to permit the biological activity of the active ingredients to be unequivocally effective, and which contain no additional components which are significantly toxic to the subjects to which the formulation would be administered.

As used herein, the term “pharmaceutically acceptable salt” represents those salts of the compounds described that are, within the scope of sound medical judgment, suitable for use in contact with the tissues of humans and animals without undue toxicity, irritation, allergic response and the like and are commensurate with a reasonable benefit/risk ratio. Pharmaceutically acceptable salts are well known in the art. For example, pharmaceutically acceptable salts are described in: Berge et al., J.

Pharmaceutical Sciences 66:1-19, 1977 and in Handbook of Pharmaceutical Salts: Properties, Selection, and Use, (Eds. P. H. Stahl and C. G. Wermuth), Wiley-VCH, 2008. These salts may be acid addition salts involving inorganic or organic acids. The salts can be prepared in situ during the final isolation and purification of the compounds described herein or separately by reacting the free base group with a suitable acid. Methods for preparation of the appropriate salts are well-established in the art. Representative acid addition salts include acetate, adipate, alginate, ascorbate, aspartate, benzenesulfonate, benzoate, bisulfate, borate, bromide, butyrate, camphorate, camphorsulfonate, chloride, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts and the like.

As used herein, the term “treating” includes therapeutic treatments. The term “therapeutic” treatment is art-recognized and includes administration to a human subject of one or more of the disclosed compounds or formulations after manifestation of the unwanted condition (i.e., it is intended to diminish, ameliorate, or stabilize the existing unwanted condition or side effects thereof). Preferably, it is intended that the severity of the subject's condition (e.g., lung dysfunction) is reduced or at least partially improved or modified and that some alleviation, mitigation, reversal or decrease in at least one clinical symptom (e.g., weight loss in subjects compared to normal subjects) is achieved.

The terms “human subject,” “subject,” and “patient” are used interchangeably herein, and refer to a subject to be treated and/or from whom a biological sample (e.g., cells) is obtained.

As used herein, “effective treatment” refers to treatment producing a beneficial effect, e.g., amelioration of at least one symptom of a disease or disorder in a subject. A beneficial effect can take the form of an improvement over baseline, i.e., an improvement over a measurement or observation made prior to initiation of therapy according to the method.

In certain embodiments, for treating a human subject with a viral disease, e.g., a patient suffering from a coronaviral disease such as COVID-19, MERS, SARS, or a disease related thereto, effective treatment may refer to alleviation of at least one symptom of the disease.

In certain embodiments, effective treatment may refer to that improves the subject's chance of survival. In certain embodiments, a disclosed method improves the life expectancy of a subject by any amount of time, including at least one day, at least one week, at least two weeks, at least three weeks, at least one month, at least two months, at least three months, at least 6 months, at least one year, at least 18 months, at least two years, at least 30 months, or at least three years, or the duration of treatment.

The term “effective amount” or “therapeutically effective amount” refers to an amount of an agent that provides the desired biological, therapeutic result. That result can be reduction, amelioration, palliation, lessening, delaying, and/or alleviation of one or more of the signs, symptoms, or causes of a disease in a subject, or any other desired alteration of a biological system. An effective amount can be administered in one or more administrations. In some embodiments, an “effective amount” is the amount of at least one CFD inhibitor (e.g., at least one of Compound 1, Compound 2, Compound 3, and Compound 4, or a pharmaceutically acceptable salt thereof) that improves a pathological outcome. In some embodiments, an “effective amount” or “therapeutically effective amount” is the amount of at least one CFD inhibitor (e.g., at least one of Compound 1, Compound 2, Compound 3, or Compound 4; or a pharmaceutically acceptable salt thereof) that improves a clinical outcome, e.g., survival of a subject by any amount of time, including at least one day, at least one week, at least two weeks, at least three weeks, at least one month, at least two months, at least three months, at least 6 months, at least one year, at least 18 months, at least two years, at least 30 months, or at least three years, or the duration of treatment.

For the terms “for example” and “such as,” and grammatical equivalences thereof, the phrase “and without limitation” is understood to follow unless explicitly stated otherwise. As used herein, the term “about” is meant to account for variations due to experimental error. All measurements reported herein are understood to be modified by the term “about,” whether or not the term is explicitly used, unless explicitly stated otherwise. As used herein, the singular forms “a,” “an,” and “the” include plural referents unless the context clearly dictates otherwise.

As used herein, the term “diagnosis” refers to methods by which a determination can be made as to whether a subject is likely to be suffering from a given disease or condition, including but not limited to complement-mediated diseases. The skilled artisan often makes a diagnosis on the basis of one or more diagnostic indicators, e.g., a marker, the presence, absence, amount, or change in amount of which is indicative of the presence, severity, or absence of the disease or condition. Other diagnostic indicators can include patient history; physical symptoms, e.g., unexplained changes in vitals, or phenotypic, genotypic or environmental factors. A skilled artisan will understand that the term “diagnosis” refers to an increased probability that certain course or outcome will occur; that is, that a course or outcome is more likely to occur in a patient exhibiting a given characteristic, e.g., the presence or level of a diagnostic indicator, when compared to individuals not exhibiting the characteristic.

The term “likelihood,” as used herein, generally refers to a probability, a relative probability, a presence or an absence, or a degree.

As used herein, the term “at risk” for a disease or disorder refers to a subject (e.g., a human) that is predisposed to experiencing a particular disease. This predisposition may be due to genetic (e.g., mutations in complement proteins) or other factors (e.g., body weight, blood type, sex, etc.). Risks can be specific to a patient or subpopulation of patients (e.g., COVID-19 patients with activated APC).

As used herein, the term “marker” refers to a characteristic that can be objectively measured as an indicator of normal biological processes, pathogenic processes or a pharmacological response to a therapeutic intervention, e.g., treatment with a complement inhibitor. Representative types of markers include, for example, molecular changes in the structure (e.g., sequence or length) or number of the marker, comprising, e.g., changes in level, concentration, activity, or properties of the marker.

The term “control,” in the context of diagnosis or assessment, refers to a reference for a test sample, such as samples obtained from healthy subjects or subjects infected by unrelated infectious agents, e.g., benign coronavirus OC43, and the like. A “reference,” refers to a sample that may or may not have a disease that are used for comparisons, which thereby provides a basis to which another sample, for example, an “experimental” sample can be compared. In screening assays, a “test sample” refers to a sample compared to the reference. The reference sample need not be disease free, such as when both the reference and test samples are obtained from the same patient, but only separated by time (e.g., reference sample being obtained before therapy versus test sample being obtained post-therapy).

The term “level” can refer to binary (e.g., absent/present), qualitative (e.g., absent/low/medium/high), or quantitative information (e.g., a value proportional to number, frequency, or concentration) indicating the presence or absence of a particular molecular species.

The terms “cell viability” and the converse, “cell death,” are used in their normal manner, e.g., a determination of living or dead cells. These measurements may be made at the level of a single cell or a population of cells. A number of analysis of the viable cells may be performed, such as quantification of the viable cells, determination of mobility of the cells, or determination of morphology of cells (e.g., external or internal membrane integrity), expression of certain markers (e.g., annexin), etc. Cell viability measurements may be used to evaluate the death or life of a specific cell type, such as GPI-AP deficient cells. They may be used to evaluate effectiveness of a drug or a test agent. Testing for cell viability usually involves looking at a single cell (e.g., using microscopy) or a cell population (e.g., using FACS).

The term “sample” refers to a composition that is obtained or derived from a subject of interest that contains a cellular and/or other molecular entity that is to be characterized and/or identified. Preferably, the sample is a “biological sample,” which means a sample that is derived from a living entity, e.g., cells, tissues, organs, in vitro engineered organs, and the like. Samples include, but not limited to, primary or 2D and 3D cultured cells or cell lines, cell supernatants, cell lysates, platelets, serum, plasma, fluids such as lymph, CSF, urine, pleural fluid, and tissue culture medium, as well as tissue extracts such as homogenized tissue and cellular extracts. Samples further include biological samples that have been manipulated after their procurement, such as by treatment with reagents (e.g., de-sialyation agents).

The term “label” as used herein refers, for example, to a compound that is detectable, either directly or indirectly. The term includes colorimetric (e.g., luminescent) labels, light scattering labels or radioactive labels. Labels include, inter alia, the commercially available fluorescein phosphoramidites.

As used herein, the term “marker” refers to a characteristic that can be objectively measured as an indicator of normal biological processes, pathogenic processes or a pharmacological response to a therapeutic intervention, e.g., treatment with a drug/medicament for COVID-19. Representative types of markers include, for example, molecular changes in the structure (e.g., length of amino acid in a protein such as C3 or C5b9, e.g., due to proteolysis) or number of the marker, including, e.g., amount deposited in a cell, or a plurality of differences, such as both the levels as well as the activity of the markers of interest. The term “marker” includes both direct and indirect phenomena. For instance, wherein the analyte is C3, the marker could be C3 itself or a downstream effect of C3, e.g., terminal complement pathway protein such as C5 and effects thereof, e.g., C5 cleavage and C5b9 deposition.

The term “complement deposition” refers to an activity or event that leads to a complement component, e.g., C5b9 and/or C3, to deposit on a target cell (e.g., blood cells such as GPI-AP deficient lymphocytes) by such a manner as to trigger a series of cascades (complement activation pathways) containing complement-related protein groups in blood. In addition, protein fragments generated by the activation of a complement can induce the migration, phagocytosis and activation of immune cells. Related downstream events include, e.g., (a) hemolysis of target cells, leading to heme release and/or anemia in blood cells; (b) C3 opsonization, including phagocytosis/extra-vascular hemolysis (EVH); (c) adhesion of opsonized cells to activated endothelium; and/or (d) activation of neutrophils and platelets.

The term “biological phenomena” as used herein refers to any processes that may be perturbed in a disease state, including, measurable changes therein in response to a test agent or a drug.

The term “screening”, as used herein, refers to an assay to assess the genotype or phenotype of a cell or cell product including, but not limited to, changes in the amount or structure or activity of a protein (e.g., levels of cleaved C3, particularly cleaved C3, and more particularly, C3 convertase activity). The assays include ELISA, BIACORE assays, activity assay (e.g., to measure C3 convertase activity), etc.

The term “compounds” used in screening include any small molecule or large molecule compounds. The term “small molecule” includes compounds that are typically smaller than 5 KDa, e.g., organic compounds, peptides, aptamers, etc. The term “large molecule” includes compounds that are typically larger than 5 KDa, e.g., proteins and antibodies. Compounds may include agents known to have desired biological effects, e.g., reduce complement deposition or inhibit cell death.

The term “attenuation” refers to the reduction of the force, effect, or value, as compared to a reference (e.g., a decrease by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95%, as compared to a reference).

The term “positive”, as used herein, refers to identification of a parameter (e.g., the expression of a marker protein or activity thereof), which greater than by at least 5% (e.g., 10%, 20%, 30%, 50%, 75%, 100%, 200%, 300%, 500%, or more, e.g., 10-fold, 20-fold or 50-fold) of a control (e.g., expression of the same protein or activity thereof in a control cell, e.g., untreated cell). The term “negative”, as used herein, refers to identification of a parameter (e.g., the expression of a protein or activity thereof), which less than 5% (e.g., 4%, 3%, 2%, 1%) of a control (e.g., expression of the same protein or activity thereof in a control cell, e.g., untreated cell).

As used herein, the terms “treat” or “treating” refer to providing an intervention, e.g., providing any type of medical or surgical management of a subject. The treatment can be provided to reverse, alleviate, inhibit the progression of, prevent or reduce the likelihood of a disorder or condition, or to reverse, alleviate, inhibit, or prevent the progression of, prevent or reduce the likelihood of one or more symptoms or manifestations (e.g., pathophysiology) of a disorder or condition. “Prevent” refers to causing a disorder or condition, or symptom or manifestation of such not to occur for at least a period of time in at least some individuals. Treating can include administering a test agent or a complement modulator (e.g., CFD inhibitor or C5 inhibitor) to the subject following the development of one or more symptoms or manifestations indicative of a complement-mediated condition, e.g., to reverse, alleviate, reduce the severity of, and/or inhibit or prevent the progression of the condition and/or to reverse, alleviate, reduce the severity of, and/or inhibit or one or more symptoms or manifestations of the condition. According to the methods described herein, a complement inhibitor (e.g., a C3 inhibitor, a CFD inhibitor, or a C5 inhibitor) can be administered to a subject who has developed disease (e.g., COVID-19) or is at increased risk of developing such a disorder relative to a member of the general population. A complement inhibitor can be administered prophylactically, i.e., before development of any symptom or manifestation of the condition.

The term “symptom” refers to an indication of disease, illness, injury, or that something is not right in the body. Symptoms are felt or noticed by the individual experiencing the symptom, but may not easily be noticed by others, e.g., non-medical professionals. The term “sign” also refers an indication that something is not right in the body, which can be seen by a doctor, nurse, or other professional.

The terms “administration” or “administering” when used in conjunction with an agent, e.g., drug, mean to deliver the agent directly into or onto a cell or target tissue or to provide the agent to a patient whereby it impacts the tissue to which it is targeted. The term “contact” refers to bringing an agent (e.g., an antibody, a nucleic acid molecule, a peptide, a small molecule, or an aptamer) and the target (e.g., C3, Factor D, or C5) in sufficiently close proximity to each other for one to exert a biological effect on the other (e.g., inhibition of the target). In some embodiments, the term contact means binding of the agent to the target. The terms “inhibitor” or “antagonist” as used herein refer to a substance, such as an antibody, nucleic acid, aptamer, and small molecule, that suppress the expression, activity, and/or level of another substance (e.g., complement C3, CFD, or C5). The term “inhibit” or “reduce” or grammatical variations thereof refers to a decrease or diminishment in the specified level or activity of the target, e.g., little or essentially no detectible level or activity of the target (at most, an insignificant amount). Examples of inhibitors of this type are antibodies, small molecules and siRNA. The term “inhibitor of complement pathway” refers to inhibitors that suppress the activation of or response of the complement pathway.

As used herein, “reducing the risk of death” refers to reducing the frequency of deaths among subjects treated according to any of the methods of the disclosure. The reduction is in comparison to control subjects (e.g., untreated subjects or subjects treated with remdesivir only) of the same age, sex, and/or condition (e.g., comorbidities). In some embodiments, the frequency of death Is reduced by at least 10% (e.g., at least 20%, at least 30%, at least 40%, at least 50%, at least 60%, at least 70%, at least 80%, at least 90%, or 99% or more) relative to the frequency of death observed for the control subjects.

As used herein, “reducing the risk of developing one or more respiratory syndrome” refers to reducing the frequency of developing one or more respiratory syndromes (e.g., one or more of those described herein) in subjects treated according to any of the methods of the disclosure. The reduction is in comparison to control subjects (e.g., untreated subjects or subjects treated with remdesivir only) of the same age, sex, and/or condition (e.g., comorbidities). In some embodiments, the frequency of developing one or more respiratory syndromes is reduced by at least 10% (e.g., at least 20%, at least 30%, at least 40%, at least 50%, at least 60%, at least 70%, at least 80%, at least 90%, or 99% or more) relative to the frequency observed for control subjects.

As used herein, “reducing the risk of developing a critical viral disease” refers to reducing the frequency of developing a critical viral disease (e.g., one or more of those described herein) in subjects treated according to any of the methods of the disclosure. The reduction is in comparison to control subjects (e.g., untreated subjects or subjects treated with remdesivir only) of the same age, sex, and/or condition (e.g., comorbidities). In some embodiments, the frequency of developing a critical viral disease is reduced by at least 10% (e.g., at least 20%, at least 30%, at least 40%, at least 50%, at least 60%, at least 70%, at least 80%, at least 90%, or 99% or more) relative to the frequency observed for control subjects.

As used herein, “reducing the risk of hospitalization” refers to reducing the frequency of hospitalization in subjects treated according to any of the methods of the disclosure, and “reducing the duration of hospitalization” refers to reducing the duration of hospitalization in subjects treated according to any of the methods of the disclosure. The reduction is in comparison to control subjects (e.g., untreated subjects or subjects treated with remdesivir only) of the same age, sex, and/or condition (e.g., comorbidities). In some embodiments, the frequency or duration of hospitalization is reduced by at least 10% (e.g., at least 20%, at least 30%, at least 40%, at least 50%, at least 60%, at least 70%, at least 80%, at least 90%, or 99% or more) relative to the frequency or duration of hospitalization observed for the control subjects.

As used herein, “reducing the risk of need for extracorporeal membrane oxygenation” refers to reducing the frequency of use of extracorporeal membrane oxygenation in subjects treated according to any of the methods of the disclosure, and “reducing the duration of need for extracorporeal membrane oxygenation” refers to reducing the duration of continuous use of extracorporeal membrane oxygenation in subjects treated according to any of the methods of the disclosure. The reduction is in comparison to control subjects (e.g., untreated subjects or subjects treated with remdesivir only) of the same age, sex, and/or condition (e.g., comorbidities). In some embodiments, the frequency or duration of continuous use of extracorporeal membrane oxygenation is reduced by at least 10% (e.g., at least 20%, at least 30%, at least 40%, at least 50%, at least 60%, at least 70%, at least 80%, at least 90%, or 99% or more) relative to the frequency or duration of continuous use of extracorporeal membrane oxygenation observed for the control subjects.

As used herein, “reducing the risk of need for invasive mechanical ventilation” refers to reducing the frequency of use of invasive mechanical ventilation (e.g., respiratory intubation or high-flow oxygen therapy) in subjects treated according to any of the methods of the disclosure, and “reducing the duration of need for invasive mechanical ventilation” refers to reducing the duration of continuous use of invasive mechanical ventilation (e.g., respiratory intubation or high-flow oxygen therapy) in subjects treated according to any of the methods of the disclosure. The reduction is in comparison to control subjects (e.g., untreated subjects or subjects treated with remdesivir only) of the same age, sex, and/or condition (e.g., comorbidities). In some embodiments, the frequency or duration of continuous use of invasive mechanical ventilation (e.g., respiratory intubation or high-flow oxygen therapy) is reduced by at least 10% (e.g., at least 20%, at least 30%, at least 40%, at least 50%, at least 60%, at least 70%, at least 80%, at least 90%, or 99% or more) relative to the frequency or duration of continued use of invasive mechanical ventilation (e.g., respiratory intubation or high-flow oxygen therapy) observed for the control subjects.

As used herein, “reducing the risk of need for non-invasive mechanical ventilation” refers to reducing the frequency of use of non-invasive mechanical ventilation (e.g., supplemental oxygen, continuous positive airway pressure (CPAP), or bilevel positive airway pressure (BiPAP)) in subjects treated according to any of the methods of the disclosure, and “reducing the duration of need for non-invasive mechanical ventilation” refers to reducing the duration of continuous use of non-invasive mechanical ventilation in subjects treated according to any of the methods of the disclosure. The reduction is in comparison to control subjects (e.g., untreated subjects or subjects treated with remdesivir only) of the same age, sex, and/or condition (e.g., comorbidities). In some embodiments, the frequency or duration of continuous use of non-invasive mechanical ventilation is reduced by at least 10% (e.g., at least 20%, at least 30%, at least 40%, at least 50%, at least 60%, at least 70%, at least 80%, at least 90%, or 99% or more) relative to the frequency or duration of continuous use of non-invasive mechanical ventilation observed for the control subjects. In some embodiments, wherein a subject is on chronic supplemental oxygen, the reduction in the risk or duration of need for supplemental oxygen pertains to a reduction in the risk or duration of an increased oxygen requirement above baseline.

As used herein, the term “reference standard” refers to a reference for a test sample, such as control healthy subjects or untreated subjects, and the like. In some embodiments, a reference standard refers to a sample of tissue or cells obtained from a subject that may or may not have an infection or disease (e.g., a betacoronavirus infection or disease) that are used for comparisons. Thus, a reference standard sample thereby provides a basis to which another sample, for example, cells from a patient with a viral infection (e.g., a betacoronavirus infection such as a SARS-CoV infection, a MERS-CoV infection, or a SARS-CoV-2 infection), can be compared.

Unless defined otherwise, all technical and scientific terms used herein have the same meaning as commonly understood by one of ordinary skill in the art to which this disclosure belongs. Specific methods, devices, and materials are described, although any methods and materials similar or equivalent to those described herein can be used in the practice or testing of the present disclosure.

The Complement System

As is well known, the complement system acts in conjunction with other immunological systems of the body to defend against intrusion of cellular and viral pathogens. There are at least 25 complement proteins. Complement components achieve their immune defensive functions by interacting in a series of intricate but precise enzymatic cleavage and membrane binding events. The resulting complement cascade leads to the production of products with opsonic, immunoregulatory, and lytic functions.

The complement cascade can progress via the classical pathway (“CP”), the lectin pathway, or the alternative pathway (“AP”). The lectin pathway is typically initiated with binding of mannose-binding lectin (“MBL”) to high mannose substrates. The AP can be antibody-independent and can be initiated by certain molecules on pathogen surfaces. The CP is typically initiated by antibody recognition of, and binding to, an antigenic site on a target cell. These pathways converge at the C3 convertase—the point where complement component C3 is cleaved by an active protease to yield C3a and C3b.

The disclosure relates to therapy of coronaviral diseases using an inhibitor of the APC, such as a CFD inhibitor (e.g., any one of Compounds 1-4, or a pharmaceutically salt thereof). Generally, the AP is initiated by C3b (generated from the activation pathways or non-specific sources) binding factor B (FB), which is then cleaved by factor D (FD) to form the C3 convertase, C3bBb. C3bBb cleaves C3 to C3b, coating adjacent surfaces and generating a C5 convertase, C3bBbC3b. Activation of C3 in the fluid phase primes the system for rapid amplification on activating surfaces, typified by absence of the regulatory proteins that suppress activation on “self” cells. FB can bind to any C3b deposited on an activating surface, including that resulting from activation of the classical and lectin pathways. Thus, the alternative pathway is known as the amplification loop of the complement cascade and plays a crucial role in amplifying any small trigger to a large downstream response.

Without being bound to a particular theory, the details of the alternative pathway, including role(s) of various components thereof, are provided below.

The AP C3 convertase is initiated by the spontaneous hydrolysis of complement component C3, which is abundant in the plasma in the blood. This process, also known as “tickover,” occurs through the spontaneous cleavage of a thioester bond in C3 to form C3i or C3(H₂O). Tickover is facilitated by the presence of surfaces that support the binding of activated C3 and/or have neutral or positive charge characteristics (e.g., bacterial cell surfaces). This formation of C3(H₂O) allows for the binding of plasma protein FB, which in turn allows FD to cleave FB into Ba and Bb. The Bb fragment remains bound to C3 to form a complex containing C3(H₂O)Bb—the “fluid-phase” or “initiation” C3 convertase. Although only produced in small amounts, the fluid-phase C3 convertase can cleave multiple C3 proteins into C3a and C3b and results in the generation of C3b and its subsequent covalent binding to a surface (e.g., a bacterial surface). FB bound to the surface-bound C3b is cleaved by FD to thus form the surface-bound AP C3 convertase complex containing C3b,Bb. See, e.g., Müller-Eberhard (1988) Ann Rev Biochem 57:321-347.

The AP C5 convertase—(C3b)₂,Bb—is formed upon addition of a second C3b monomer to the AP C3 convertase. See, e.g., Medicus et al. (1976) J Exp Med 144:1076-1093 and Fearon et al. (1975) J Exp Med 142:856-863. The role of the second C3b molecule is to bind C5 and present it for cleavage by Bb. See, e.g., Isenman et al (1980) J Immunol 124:326-331. The AP C3 and C5 convertases are stabilized by the addition of the trimeric protein properdin as described in, e.g., Medicus et a. (1976), supra. However, properdin binding is not required to form a functioning alternative pathway C3 or C5 convertase. See, e.g., Schreiber et al. (1978) Proc Natl Aced Sci USA 75: 3948-3952, and Sissons et al. (1980) Proc Natl Acad Sci USA 77: 559-562.

The CP C3 convertase is formed upon interaction of complement component C1, which is a complex of C1q, C1r, and C1s, with an antibody that is bound to a target antigen (e.g., a microbial antigen). The binding of the C1q portion of C1 to the antibody-antigen complex causes a conformational change in C1 that activates Cir. Active Cir then cleaves the C1-associated C1s to thereby generate an active serine protease. Active C1s cleaves complement component C4 into C4b and C4a. Like C3b, the newly generated C4b fragment contains a highly reactive thiol that readily forms amide or ester bonds with suitable molecules on a target surface (e.g., a microbial cell surface). C1s also cleaves complement component C2 into C2b and C2a. The complex formed by C4b and C2a is the CP C3 convertase, which is capable of processing C3 into C3a and C3b. The CP C5 convertase—C4b, C2a, C3b—is formed upon addition of a C3b monomer to the CP C3 convertase. See, e.g., Müller-Eberhard (1988), supra and Cooper et al. (1970) J Exp Med 132:775-793.

In addition to its role in C3 and C5 convertases, C3b also functions as an opsonin through its interaction with complement receptors present on the surfaces of antigen-presenting cells such as macrophages and dendritic cells. The opsonic function of C3b is generally considered to be one of the most important anti-infective functions of the complement system. Patients with genetic lesions that block C3b function are prone to infection by a broad variety of pathogenic organisms, while patients with lesions later in the complement cascade sequence, i.e., patients with lesions that block C5 functions, are found to be more prone only to Neisseria infection, and then only somewhat more prone.

The AP and CP C5 convertases cleave C5, which is a 190 kDa beta globulin found in normal human serum at approximately 75 μg/ml (0.4 μM). C5 is glycosylated, with about 1.5-3 percent of its mass attributed to carbohydrate. Mature C5 is a heterodimer of a 999 amino acid 115 kDa alpha chain that is disulfide linked to a 655 amino acid 75 kDa beta chain. C5 is synthesized as a single chain precursor protein product of a single copy gene (Haviland et al. (1991) J Immunol. 146:362-368). The cDNA sequence of the transcript of this human gene predicts a secreted pro-C5 precursor of 1658 amino acids along with an 18 amino acid leader sequence. See, e.g., U.S. Pat. No. 6,355,245.

The pro-C5 precursor is cleaved after amino acids 655 and 659, to yield the beta chain as an amino terminal fragment (amino acid residues +1 to 655 of the above sequence) and the alpha chain as a carboxyl terminal fragment (amino acid residues 660 to 1658 of the above sequence), with four amino acids (amino acid residues 656-659 of the above sequence) deleted between the two.

C5a is cleaved from the alpha chain of C5 by either alternative or classical C5 convertase as an amino terminal fragment comprising the first 74 amino acids of the alpha chain (i.e., amino acid residues 660-733 of the above sequence). Approximately 20 percent of the 11 kDa mass of C5a is attributed to carbohydrate. The cleavage site for convertase action is at, or immediately adjacent to, amino acid residue 733. A compound that would bind at, or adjacent to, this cleavage site would have the potential to block access of the C5 convertase enzymes to the cleavage site and thereby act as a complement inhibitor. A compound that binds to C5 at a site distal to the cleavage site could also have the potential to block C5 cleavage, for example, by way of steric hindrance-mediated inhibition of the interaction between C5 and the C5 convertase. A compound, in a mechanism of action consistent with that of the tick saliva complement inhibitor, Omithodoros moubata C inhibitor (‘OmCl”) (which can be a C5 inhibitor that can be used in the methods of this disclosure), may also prevent C5 cleavage by reducing flexibility of the C345C domain of the alpha chain of C5, which reduces access of the C5 convertase to the cleavage site of C5. See, e.g., Fredslund et al. (2008) Nat Immunol 9(7):753-760.

C5 can also be activated by means other than C5 convertase activity. Limited trypsin digestion (see, e.g., Minta and Man (1997) J Immunol 119:1597-1602 and Wetsel and Kolb (1982) J Immunol 128:2209-2216) and acid treatment (Yamamoto and Gewurz (1978) J Immunol 120:2008 and Damerau et al. (1989) Molec Immunol 26:1133-1142) can also cleave C5 and produce active C5b.

Cleavage of C5 releases C5a, a potent anaphylatoxin and chemotactic factor, and leads to the formation of the lytic terminal complement complex. C5b-9, C5a, and C5b-9 also have pleiotropic cell activating properties, by amplifying the release of downstream inflammatory factors, such as hydrolytic enzymes, reactive oxygen species, arachidonic acid metabolites and various cytokines.

The first step in the formation of the terminal complement complex involves the combination of C5b with C6, C7, and C8 to form the C5b-8 complex at the surface of the target cell. Upon the binding of the C5b-8 complex with several C9 molecules, the membrane attack complex (“MAC”, C5b-9, terminal complement complex—“TCC”) is formed. When sufficient numbers of MACs insert into target cell membranes the openings they create (MAC pores) mediate rapid osmotic lysis of the target cells, such as red blood cells. Lower, non-lytic concentrations of MACs can produce other effects. In particular, membrane insertion of small numbers of the C5b-9 complexes into endothelial cells and platelets can cause deleterious cell activation. In some cases, activation may precede cell lysis.

C3a and C5a are anaphylatoxins. These activated complement components can trigger mast cell degranulation, which releases histamine from basophils and mast cells, and other mediators of inflammation, resulting in smooth muscle contraction, increased vascular permeability, leukocyte activation, and other inflammatory phenomena including cellular proliferation resulting in hypercellularity. C5a also functions as a chemotactic peptide that serves to attract pro-inflammatory granulocytes to the site of complement activation.

C5a receptors are found on the surfaces of bronchial and alveolar epithelial cells and bronchial smooth muscle cells. C5a receptors have also been found on eosinophils, mast cells, monocytes, neutrophils, and activated lymphocytes.

While a properly functioning complement system provides a robust defense against infecting microbes, inappropriate regulation or activation of complement has been implicated in the pathogenesis of a variety of disorders, including, e.g., rheumatoid arthritis; lupus nephritis; asthma; ischemia-reperfusion injury; atypical hemolytic uremic syndrome (“aHUS”); dense deposit disease; paroxysmal nocturnal hemoglobinuria (PNH); macular degeneration (e.g., age-related macular degeneration; hemolysis, elevated liver enzymes, and low platelets (HELLP) syndrome; thrombotic thrombocytopenic purpura (TTP); spontaneous fetal loss; Pauci-immune vasculitis; epidermolysis bullosa; recurrent fetal loss; multiple sclerosis (MS); traumatic brain injury; and injury resulting from myocardial infarction, cardiopulmonary bypass and hemodialysis. See, e.g., Holers et al. (2008) Immunological Reviews 223:300-316.

Coronaviral Diseases

The disclosure relates to treatment of coronaviral disease in a subject comprising administering an effective amount of at least one CFD inhibitor (e.g., at least one of Compound 1, Compound 2, Compound 3, and Compound 4; or a pharmaceutically acceptable salt thereof). Preferably, the compositions containing at least one CFD inhibitor (e.g., at least one of Compound 1, Compound 2, Compound 3, and Compound 4; or a pharmaceutically acceptable salt thereof) are useful in the treatment of diseases elicited by coronaviruses such as SARS coronavirus (SARS-CoV), MERS coronavirus (MERS-CoV), COVID-19 coronavirus (2019-nCoV or SARS-CoV-2) or a coronavirus related thereto.

Coronaviruses are enveloped viruses having a capsid having a helical symmetry. They have a positive-sense single-stranded RNA genome and can infect the cells of birds and mammals. The viruses belonging to this very large family are known to be causative agents of colds (for example the hCoV and OC43 viruses), bronchiolitis (for example the NL63 virus) or even certain forms of severe pneumonia such as those observed during SARS epidemic (such as SARS-CoV).

Despite their belonging to the same viral family, important differences exist between the different coronaviruses, both at the genetic and structural level, but also in terms of biology and sensitivity to antiviral molecules. See, e.g., Dijkman et al. (J Formos Med Assoc. 2009 April; 108(4):270-9; PMID: 19369173); de Wit et al. (Nat Rev Microbiol. 2016 August; 14(8):523-34; PMID: 27344959).

SARS-CoV

SARS-CoV is a species of coronavirus known to infect certain mammals such as humans. Two strains of the virus have caused outbreaks of severe respiratory diseases in humans: SARS-CoV, which caused an outbreak of severe acute respiratory syndrome (SARS) between 2002 and 2004, and SARS-CoV-2, which since late 2019 has caused an outbreak of coronavirus disease 2019 (COVID-19). Both strains descended from a single ancestor but made the cross-species jump into humans separately. It is thought that SARS-CoV-2 is not a direct descendant of SARS-CoV (Gorbalenya et al., Feb. 11, 2020; world-wide-web at biorxiv(dot)org/content/10.1101/2020.02.07.937862v1). There are hundreds of other strains of SARS-CoV, most of which are only known to infect non-human species: bats are a major reservoir of many strains of SARS-like coronaviruses, and several strains have been identified in palm civets which were likely ancestors of SARS-CoV.

An epidemic of SARS affected 26 countries and resulted in more than 8000 cases in 2003 (WHO Report, 2020). Since then, a small number of cases have occurred as a result of laboratory accidents or, possibly, through animal-to-human transmission. Symptoms of SARS are influenza-like and include fever, malaise, myalgia, headache, diarrhea, and shivering (rigors). No individual symptom or cluster of symptoms has proved to be specific for a diagnosis of SARS. Although fever is the most frequently reported symptom, it is sometimes absent on initial measurement, especially in elderly and immunosuppressed patients. Cough (initially dry), shortness of breath, and diarrhea are present in the first and/or second week of illness. Severe cases often evolve rapidly, progressing to respiratory distress and requiring intensive care.

SARS is transmitted by aerosols of respiratory secretions, by the fecal-oral route, and by mechanical transmission. Most virus growth occurs in epithelial cells. Occasionally the liver, kidneys, heart or eyes may be infected, as well as other cell types such as macrophages. Transmission of SARS-CoV is primarily from person to person. It appears to have occurred mainly during the second week of illness, which corresponds to the peak of virus excretion in respiratory secretions and stool, and when cases with severe disease start to deteriorate clinically. Most cases of human-to-human transmission occurred in the health care setting, in the absence of adequate infection control precautions. Implementation of appropriate infection control practices brought the global outbreak to an end.

In cold-type respiratory infections, growth appears to be localized to the epithelium of the upper respiratory tract. Clinically, most infections cause a mild, self-limited disease (classical “cold” or upset stomach), but there may be rare neurological complications. The disease results in death in about 3 to 10% of cases.

Laboratory diagnosis of SARS can be carried out using ELISA, complement fixation or hemagglutination tests. Growth in culture is usually ineffective for coronavirus isolation. Since the complete genome of SARS-CoV (as well as common variants thereof) have been identified, genetic testing may be used for diagnosis. The genome of SARS-CoV is a 29,727-nucleotide polyadenylated RNA, has 11 open reading frames, and 41% of the residues are G or C. The genomic organization is typical of coronaviruses, with the characteristic gene order (5′-replicase (rep), spike (S), envelope (E), membrane (M), nucleocapsid (N)-3′ and short untranslated regions at both termini. The SARS-CoV rep gene, which comprises about two-thirds of the genome, is predicted to encode two polyproteins that undergo co-translational proteolytic processing. There are four open reading frames (ORFs) downstream of rep that are predicted to encode the structural proteins, S, E, M and N, which are common to all known coronaviruses. The hemagglutinin-esterase gene, which is present between ORFlb and S in group 2 and some group 3 coronaviruses was not found. Phylogenetic analyses and sequence comparisons showed that SARS-CoV is not closely related to any of the previously characterized coronaviruses. Other techniques for detection of bioagents include high-resolution mass spectrometry (MS), low-resolution MS, fluorescence, radioiodination, DNA chips and antibody techniques.

MERS-CoV

The MERS-CoV is a new emerging virus identified in 2012 in Saudi Arabia, responsible for SARS and kidney failure. Since its identification, this virus has been responsible for more than 1,806 cases of infection in 26 countries, mainly in the Middle East. It is responsible for 643 deaths or nearly 35.6% mortality according to the World Health Organization (Source WHO, Sep. 28, 2016).

The MERS-CoV belongs to the order of Nidovirales, to the family of Coronaviridae, and to the genus Betacoronavirus. Although most cases of MERS-CoV in humans are attributable to human-to-human transmission, the camel appears to be a permanent intermediate infected animal host of MERS-CoV and thus constitutes the main animal source of infection in humans.

There is currently no therapeutic solution to effectively treat this epidemic respiratory viral pathogen with pandemic potential. Several therapeutic avenues have recently been explored: use of ribavirin, interferon, or mycophenolic acid. Unfortunately, most of these compounds have not shown enough efficacy when used in infected patients (Al-Tawfiq et al., Int J Infect Dis. 2014 March; 20:42-6; PMID: 24406736) or as part of prophylactic treatment (de Wit et al., 2016, supra).

A first strategy for therapy against MERS-CoV was to test, among the many known antiviral molecules, those used to combat SARS-CoV. Thus, inhibitors of viral replication, such as protease inhibitors, helicase inhibitors, and inhibitors of entry of the virus into the target cells were tested in vitro. Dyall et al. (Antimicrob Agents Chemother. 2014 August; 58(8):4885-93; PMID: 24841273) tested different categories of drugs with the aim of identifying anti-viral agents active on the SARS and/or MERS-COV coronaviruses. Among the different classes of agents tested, it was shown that certain anti-inflammatory agents inhibited the proliferation of SARS-CoV, while MERS-CoV was rather inhibited by certain inhibitors of ion transport, inhibitors of tubulin, or apoptosis inhibitors. Out of 290 compounds tested, only 33 compounds with antiviral activity on MERS-CoV were identified in cell culture.

However, owing partly to differences in terms of both protein composition and functional interactions with the host cell, many antiviral compounds effective on SARS-CoV are not systematically active against MERS-CoV, and vice versa. Also, currently, there are no, or very few, therapeutic molecules recognized and/or approved by health authorities to fight infections with the MERS-CoV virus. In addition, there is no vaccine on the market against the MERS-CoV virus. Some candidates are being evaluated in a phase I clinical trial, with ongoing efficacy evaluations (National Clinical Trials #NCT02670187). See, Modjarrad et al. (Lancet Infect Dis. 2019 September; 19(9):1013-1022; PMID: 31351922).

Effect of Coronavirus in the Respiratory System

SARS-CoV infection in humans results in an acute respiratory illness that varied from mild febrile illness to ALI and, in some cases, ARDS and death. See, Channappanavar et al. (Semin Immunopathol. (Review) 2017 July; 39(5):529-539; PMID: 28466096). The clinical course of SARS presents in three distinct phases—(a) an initial phase characterized by robust virus replication accompanied by fever, cough, and other symptoms, all of which subsided in a few days; (b) a second clinical phase associated with high fever, hypoxemia, and progression to pneumonia-like symptoms, with declining virus titers towards the end of this phase; and (c) a third phase in which patients progress to ARDS, often resulting in death. The third phase is thought to have resulted from exuberant host inflammatory responses.

The most common clinical manifestations of MERS include flu-like symptoms such as fever, sore throat, non-productive cough, myalgia, shortness of breath, and dyspnea, which rapidly progress to pneumonia. See, Channappanavar et al. (supra). Other atypical presentations include mild respiratory illness without fever, chills, wheezing, and palpitations. MERS-CoV in humans also causes gastrointestinal symptoms such as abdominal pain, vomiting, and diarrhea. Most MERS patients with dyspnea progress to develop severe pneumonia and require admission to an intensive care unit (ICU). Although most healthy individuals present with mild-moderate respiratory illness, immunocompromised and individuals with comorbid conditions experience severe respiratory illness, which often progressed to ARD. Overall, MERS-CoV caused severe disease in primary index cases, immunocompromised individuals and in patients with comorbid conditions, but secondary cases of household contacts or healthcare workers were mostly asymptomatic or showed mild respiratory illness.

Typically, analyses of lungs from patients who succumbed to SARS showed lung consolidation and edema with pleural effusions, focal hemorrhages, and mucopurulent material in the tracheobronchial tree. Diffuse alveolar damage (DAD) was a prominent histological feature in SARS lungs. Other changes included hyaline membrane formation, alveolar hemorrhage, and fibrin exudation in alveolar spaces with septal and alveolar fibrosis observed during later stages. Staining for viral antigen revealed infection of airway and alveolar epithelial cells, vascular endothelial cells, and macrophages. Furthermore, SARS-CoV viral particles and viral genome were also detected in monocytes and lymphocytes. See, Gu et al. (J Exp Med. 2005 Aug. 1; 202(3):415-24); Nicholls et al. (Lancet. 2003 May 24; 361(9371):1773-8). In addition to these changes, histological examination of lungs from patients who died of SARS revealed extensive cellular infiltrates in the interstitium and alveoli. These cellular infiltrates included neutrophils and macrophages with macrophages being the predominant cell type. These results correlated with increased numbers of neutrophils and monocytes and lower CD4 and CD8 T cell counts in the peripheral blood samples of patients with fatal SARS.

With respect to MERS, analysis of lung tissue from human patient showed pleural, pericardial, and abdominal effusions associated with generalized congestion, edema, and consolidation of lungs (Ng et al., Am J Pathol. 2016 March; 186(3):652-8). Similar to SARS-CoV infection, DAD was a prominent feature in the lungs. Additionally, epithelial cell necrosis, sloughing of bronchiolar epithelium, alveolar edema, and thickening of alveolar septa were also noted. Immunohistochemical examination showed that MERS-CoV predominantly infected airways and alveolar epithelial cells, and endothelial cells and macrophages. The severity of lung lesions correlated with extensive infiltration of neutrophils and macrophages in the lungs and higher numbers of these cells in the peripheral blood of MERS patients.

With respect to etiological agents contributing to lung injury in patients afflicted with pathogenic coronaviruses such as SARS and MERS, cytokines and chemokines have long been thought to play an important role in immunity and immunopathology during virus infections. A rapid and well-coordinated innate immune response is the first line of defense against viral infections, but dysregulated and excessive immune responses may cause immunopathology (Channappanavar et al., supra). Although there is no direct evidence for the involvement of pro-inflammatory cytokines and chemokines in lung pathology during SARS and MERS, correlative evidence from patients with severe disease suggests a role for hyper-inflammatory responses in hCoV pathogenesis.

The risk of damage in the pulmonary system due to new types of coronaviral infection is grave. Notably, metagenomics and synthetic virus recovery strategies have since revealed the existence of large pools of pre-epidemic SARS-like bat coronaviruses which replicate in primary human airway epithelial cells. These viruses are poised for emergence because they both efficiently use human ACE2 entry receptors and resist existing vaccines and immunotherapeutics (Menachery et al., Nat Med. 2015 December; 21(12):1508-1; PMID: 26552008). New, highly pathogenic coronaviruses from animal reservoirs are likely to emerge in the future. Many new members of SARS-CoV and MERS-CoV continue to cause a range of effects on the lung tissue, ranging from asymptomatic cases to severe acute respiratory distress syndrome (ARDS) and respiratory failure (Hui et al., Cuff Opin Pulm Med. 2014 May; 20(3):233-41; PMID: 24626235).

Diagnostic Methods

The present disclosure provides methods for testing a patient for mutations in complement related genes, specifically, for mutations in genes that inhibit regulation of APC or mutations that directly activate APC. Patients can be tested for mutations in complement factor H (CFH), CFH-related proteins (CFHR1, CFHR2, CFHR3, CFHR4, CFHR5), complement factor I (CFI), CD46 (membrane cofactor protein, MCP), complement factor B (CFB), complement component C3 (C3), thrombomodulin (THBD), plasminogen, diacylglycerolkinase-E (DGKE), complement factor D (CFD), and complement receptor 1 (CR1).

In more particular embodiments, patients are tested for loss of function mutation(s) in a complement inhibitory factor (CFH, CFI, CD46 (MCP), THBD, CR1) or a gain of function mutation(s) of a complement activating factor (CFB, C3). Such mutations are known in the art. These patients are likely to be predisposed to uncontrolled complement activation, which could lead to disseminated thrombosis and multi-organ failure in the setting of a complement amplifying trigger such as infection (COVID-19), as well as surgery, pregnancy, or autoimmune disease.

In some embodiments, depending on the mutation(s), a patient infected with SARS-CoV-2 (e.g., a COVID-19 patient) may benefit from a terminal complement inhibitor (e.g., anti-C5 antibody (eculizumab)) or an APC inhibitor such as a CFD inhibitor (e.g., one or more of Compounds 1-4) or a Factor B inhibitor (IONIS-FB-LRx), as well as administration of Factor H.

In particular embodiments, the present disclosure utilizes glycosylphosphatidylinositol-anchored protein (GPI-AP) deficient cells. In some embodiments, cells are biochemically treated to remove GPI-AP. In other embodiments, the plurality of GPI-AP deficient cells is a phosphatidylinositol glycan class A (PIGA) null mutant cell line. The present inventors previously established a PIGA mutant cell line derived from TF1 cells. PIGA is a gene required for the first step in the biosynthesis of glycosylphosphatidylinositol (GPI), a lipid moiety that anchors dozens of proteins to the cell surface. Two of the GPI-anchored proteins that are defective in the TF1 cell line are CD55 and CD59. The proteins both regulate complement. CD55 blocks C3 convertases and CD59 interferes with/blocks terminal complement activation.

In particular embodiments, the compositions and methods of the present disclosure use this cell line as a reporter cell line for activation of complement in patient serum. In certain embodiments, a flow cytometry assay is performed as described herein. In other embodiments, a modified Ham assay is performed. Briefly, about 5 cc of serum is collected from patients, diluted 1:4 with growth medium and viability of the PIGA mutant TF1 cells is measured after 30 minutes using a WST1 assay. To confirm that the cell kill is associated with complement, the cells are stained with a monoclonal antibody to C5b9 (terminal complement attack) and assay the staining by flow cytometry.

In a non-limiting embodiment, the modified Ham assay may be conducted as follows: Blood is collected in serum separation tubes and is immediately centrifuged at 4° C. Serum is separated and stored at −80° C. Heat inactivation is performed the same day of the experiment, incubating the serum at 56° C. for 30 minutes.

In certain embodiments, the cell viability assay is performed on a glycosylphosphatidylinositol-anchored proteins (GPI-AP) deficient TF-1 cell line that has been previously established. See Savage et al., 37(1) EXP. HEMATOL. 42-51 (2009). Cells are maintained in RPMI 1640 medium supplemented with 2 ng/mL GM-CSF, 2 mM 1-glutamine, penicillin/streptomycin, and 10% fetal calf serum under BL2 lab containment.

Cells are plated in a U-shaped 96-well plate at a density of approximately 4,000 cells/well and cultured until confluent. Then, cells are washed with PBS and incubated with serum at a concentration of 1:4 for 30 minutes at 37° C. Serum is diluted in GVB (gelatin veronal buffer, Sigma). Cells are washed again with PBS and incubated with the cell proliferation reagent (4-[3-(4-Iodophenyl)-2-(4-nitrophenyl)-2H-5-tetrazolio]-1.3-benzene disulfonate/WST-1, Roche) for 3 hours at 37° C. Wst-1 is diluted in the cell culture medium at a concentration of 1:10 and 100 μl of Wst-1 solution is added per well. Absorbance is measured in a microplate (ELISA) reader at 450 nm with a reference wavelength at 650 nm, according to the manufacturer's instructions and previous publication. See Taylor et al., 23(4) PEDS 251-60 (2010). The colorimetric assay is based on cleavage of the tetrazolium salt, WST-1, by mitochondrial dehydrogenases in viable cells.

In certain embodiments, absorbance values of each sample are normalized after subtraction of the absorbance value of a blank cell. Percentage of viable cells is expressed as a ratio of the absorbance of each sample multiplied by 100, to the absorbance of the same sample's heat-inactivated control. Percentage of dead cells is calculated after subtracting percentage of viable cells from 100.

The cell viability indicator can be any substance, composition or compound capable of providing a particular change which selectively identifies the presence of viable cells in the biological sample. In particular embodiments, the cell viability indicator is a tetrazole. Tetrazoles serve as a substrate for an enzymatic reaction, which provides a colorimetric measure of the activity of cellular metabolic enzymes that reduce the tetrazoles to formazan. Such tetrazoles include, but are not limited to, 3-(4,5-Dimethylthiazol-2-yl)-2,5-diphenyltetrazolium bromide (MTT), 2,3-bis-(2-methoxy-4-nitro-5-sulfophenyl)-2H-tetrazolium-5-carboxanilide (XTT), 3-(4,5-dimethylthiazol-2-yl)-5-(3-carboxymethoxyphenyl)-2-(4-sulfo-phenyl)-2H-tetrazolium (MTS) or Water soluble Tetrazolium salts (WTSs), for example WST-1 (2-(4-Iodophenyl)-3-(4-nitrophenyl)-5-(2,4-disulfophenyl)-2H-tetrazolium) and WST-8 (2-(2-methoxy-4 nitrophenyl)-3-(4-nitrophenyl)-5-(2,4-disulfophenyl)-2H-tetrazolium).

Other suitable cell viability indicator reagents may also be used. In certain embodiments of the disclosure, biological samples are exposed to fluorescent dyes to provide information regarding the biological function of the cells within the sample. Such fluorescent dyes include “live cell” dyes (e.g., calcein AM) which selectively accumulate within viable cells and which are modified within the environment of viable cells to produce fluorescent chemical species. Such “live cell” dyes selectively render viable cells fluorescent whilst leaving non-viable cells unstained. Variants of these “live cell” dyes have chemical groups such that they become covalently attached to cellular proteins during fixation so that the dye is retained within the cell for prolonged periods of time. Other fluorescent dyes include “dead cell” dyes (e.g., propidium iodide or ethidium bromide homodimer) which can enter and stain non-viable cells but which are excluded from viable cells.

In further embodiments, assays that are based on the incorporation of labeled nucleotide or nucleotide analogs into the DNA of cells can be used. In such assays, cells are exposed to a labeled nucleotide, e.g., ¹⁴C-thymidine, ³H-thymidine, or 5-bromo-2-deoxyuridine (BrdU). Proliferation is quantified by measuring the amount of labeled nucleotide taken up by the cells. Radiolabeled nucleotides can be measured by radiodetection methods; antibodies can be used to detect incorporation of BrdU.

Still other assays measure cellular viability/proliferation as a function of ATP production. For example, the luciferase enzyme catalyzes a bioluminescent reaction using the substrate luciferin. The amount of bioluminescence produced by a sample of cells measures the amount of ATP present in the sample, which is an indicator of the number of cells.

In specific embodiments, the assay is repeated using complement inhibitors and noting its effect on cell viability. In other embodiments, flow cytometry is used to measure C5b9 deposition on cell membranes.

The present disclosure also provides kits for performing the assays described herein. In particular embodiments, the kit comprises a GPI-AP deficient cell line. In another embodiment, the kit can also comprise growth media for the cell line. The kit can further comprise a substrate or support for containing the cells. In other embodiments, the kit comprises a positive and negative control. The kit can also comprise the necessary buffers for preparing, washing, etc. of the samples and/or cells. In a specific embodiment, the kit also comprises the components for conducting the cell viability assay including the cell proliferation reagent (e.g., WST-1), cell viability indicator reagent and the like. In other embodiments, the kit comprises the components for conducting flow cytometry to measure C5b9 deposition on cell membranes. For example, the kit can comprise anti-C5b9 antibody. The kit can further comprise secondary antibody and labels (which could be conjugated to the primary and/or secondary antibodies). In another embodiment, the kit can comprise anti-C3c antibodies.

The present disclosure incorporates by reference the disclosures of U.S. patent application Ser. No. 15/553,836 (U.S. Patent Application Publication No. 2018/0248082) and WO2021113822A1.

Treatment of Viral Diseases

In some embodiments, the compositions containing modulators of complement pathway are useful in the treatment of diseases elicited by viruses (e.g., betacoronaviruses such as SARS-CoV, MERS-CoV, and SARS-CoV-2) which stimulate complement activation in their host subjects, e.g., betacoronaviral diseases such as SARS, MERS, COVID-19, or a disease related thereto. More specifically, because terminal complement proteins have been found to be involved in complement-mediated tissue damage triggered by viral infection, CFD inhibitors, an upstream complement inhibitor, is particularly useful in the therapy of viral diseases or symptoms related thereto.

In some embodiments, a composition containing at least one CFD inhibitor, (e.g., at least one of Compound 1, Compound 2, Compound 3, and Compound 4; or a pharmaceutically acceptable salt thereof) is useful in the amelioration of symptoms or effects of viral infection, e.g., a betacoronavirus infection such as a SARS-CoV, MERS-CoV, and SARS-CoV-2 infection.

The recalcitrant hypercoagulability, thrombotic microangiopathy, diffuse endothelial damage, and the inflammatory milieu associated with severe COVID-19 infection has led to the hypothesis that excessive complement activation may be responsible for the end-organ damage, but the mechanism is unclear (Gavriilaki et al., Br J Haematol. 2020; Campbell et al., Circulation. 2020; 141(22):1739-1741; Risitano et al., Nat Rev Immunol. 2020; 20(6):343-344). Indeed, complementopathies, such as atypical hemolytic uremic syndrome (aHUS) and catastrophic antiphospholipid antibody syndrome (CAPS) share many clinical features with severe COVID-19 infection (Chaturvedi et al. Blood. 2020; 135(4):239-251; Gavriilaki et al., J Clin Invest. 2020).

Preclinical data has also demonstrated a role for complement activation in coronavirus (CoV)-mediated disease. Gralinski et al. evaluated the activation of the complement system in a mouse model of CoV (Gralinski, 2018, supra)). Relative to C57BL/6J control mice, SARS-CoV-infected C3−/− mice exhibited significantly less weight loss and less respiratory dysfunction despite equivalent viral loads in the lung. Transgenic animals lacking C3 also had reduced inflammatory cells in the large airway and parenchyma, improved respiratory function, and lower levels of inflammatory cytokines or chemokines in the lung and periphery (Jiang et al., Emerg Mecrobes Infect. 2018; 7(1):77). Middle Eastern Respiratory Syndrome (MERS)-CoV infection in mice causes severe acute respiratory failure and high mortality accompanied by elevated secretion of cytokines and chemokines. Increased concentrations of C5a and C5b-9 (terminal complement complex), activation products resulting from cleavage of C5, were detected in sera and lung tissue in the infected mice, respectively.

In the context of COVID-19, clinical manifestations in patients show damage to vital organs such as lungs, hearts, and kidneys. Aberrant complement activation and the concomitant aggravated inflammatory lung injury has been observed in COVID-19 patients (Gao et al., supra). In humans with COVID-19 pneumonia, C5b-9, C4d, and mannan-binding lectin serine protease (MASP) 2 are found in the microvasculature of the lung, and COVID-19 associated skin lesions exhibit co-localization of SARS-CoV-2 spike proteins with C4d and C5b-9 in the cutaneous microvasculature (Magro et al., Transl Res. 2020). A prospective cohort study of 150 patients with COVID-19 acute respiratory distress syndrome (ARDS) found a high incidence of pulmonary emboli (17%) despite prophylactic anticoagulation (Helm et al., Intensive Care Med. 2020).

Elevated cytokine release has also been observed in COVID-19 patients, which is postulated to play a role in organ failure. Particularly, clinical and experimental models of COVID-19 suggest that the abnormal presence of complement components in the tubular lumen of kidneys leads to the assembly of the complement C5b-9 on the apical brush border of tubular epithelial cells (TECs), and that this is an important factor in the pathogenesis of tubulointerstitial damage (Diao et al., medRxiv, DOI: 10.1101/2020.03.04.20031120, Apr. 10, 2020). Further, in patients with severe or fatal COVID-19, there is also evidence of end organ damage with acute cardiac injury and primarily mildly elevated troponin. Cardiac dysfunction is thought to be mediated via elevated D-dimer, elevated lactate dehydrogenase, elevated total bilirubin, and decreased platelets (Campbell, 2020, supra). Death among COVID-19 patients was significantly correlated to cardiac injury, as indicated by elevated troponin levels (average troponin I of 0.19 μg/L) (51.2% vs 4.5%, respectively).

As described in Example 1, the SARS-CoV-2 spike protein subunits, but not N proteins or spike proteins from a more benign human coronavirus OC43, are potent activators of the APC and that C5 and factor D inhibitors prevent the complement-mediated damage. More specifically, SARS-CoV-2 spike proteins convert non-activator surfaces to activator surfaces by preventing the inactivation of the cell surface APC convertase. APC activation may explain many of the clinical manifestations (microangiopathy, thrombocytopenia, renal injury, and thrombophilia) of COVID-19 that are also observed in other complement-driven diseases such as atypical hemolytic uremic syndrome and catastrophic antiphospholipid antibody syndrome. C5 inhibition prevents accumulation of C5b-9 in vitro but does not prevent upstream complement activation in response to SARS-CoV-2 spike proteins.

The present disclosure also relates to combined diagnosis and treatment of COVID-19 in patients or select patient populations (e.g., COVID-19 patients with activated APC and/or COVID-19 patients with mutations, e.g., gain-of-function (GOF) or loss-of-function (LOF) mutations, in one or more complement proteins).

Embodiments of the disclosure relate to treating patients with SARS-CoV-2 infection who are identified to have activated APC. In such embodiments, classifying patients with SARS-CoV-2 infection based on activated APC status involves (a) incubating serum obtained from the patient and optionally control serum with a plurality of glycosylphosphatidylinositol-anchored protein (GPI-AP) deficient cells; (b) measuring cell viability of GPI-AP deficient cells in the incubate of (a); (c) comparing the cell viability of GPI-AP deficient cells incubated with the patient's serum with (1) a reference standard or (2) the viability of GPI-AP deficient cells incubated with the control serum, wherein if the cell viability of GPI-AP cells in the patient serum incubate is reduced compared to viability thereof in the control serum incubate or the reference standard, then the patient is classified as having activated APC (APC+SARS-CoV-2 infection) and if the cell viability of GPI-AP cells in the patient serum incubate is unchanged or elevated compared to viability thereof in the control serum incubate or the reference standard, then the patient is classified as having infection with baseline APC. The above method may be used in identifying patients who exhibit or are yet to exhibit one or more signs or symptoms of COVID-19. The disclosure also provides a method for treatment of a patient with SARS-CoV-2 infection who has activated APC, wherein the activated APC status is determined according to the foregoing method, and administering a complement modulator to the patient with activated APC (APC+SARS-CoV-2 infection).

Further embodiments of the disclosure provide methods for assessing a risk of a patient with SARS-CoV-2 infection to develop severe vascular complications, e.g., disseminated thrombosis and/or multi-organ failure, comprising (a) detecting the presence or absence of one or more mutations in complement proteins in a cell sample (e.g., blood cell sample) obtained from the patient; and (b) identifying the patient as being at high risk for developing the vascular complication if the patient's cell sample comprises a mutation in the complement protein, e.g., gain of function (GOF) mutation in the complement activating factor and/or a loss of function (LOF) mutation in the complement inhibitory factor. More specifically, the patient is categorized as high risk of developing severe vascular complications if the patient's cell sample comprises a GOF mutation in a complement activating factor selected from complement C3 and/or complement factor B and/or if the patient's cell sample comprises a LOF mutation in a complement inhibiting factor selected from complement factor H (CFH); a CFH-related protein selected from complement factor H receptor 1 (CFHR1), complement factor H receptor 2 (CFHR2), complement factor H receptor 3 (CFHR3), complement factor H receptor 1 (CFHR4), or complement factor H receptor 5 (CFHR5); complement factor I (CFI); membrane cofactor protein (MCP or CD48); thrombomodulin (THBD); and complement receptor 1 (CR1). Absence or attenuated levels of one or more of the aforementioned GOF and/or LOF mutations in the patient's sample, compared to a reference standard is indicative that the patient has moderate or even low risk of developing the severe vascular complications. Related embodiments of the disclosure provide methods for treating high risk patients, e.g., via administration of complement modulators. for treating a patient with SARS-CoV-2 infection who is at risk for developing severe vascular complications, e.g., disseminated thrombosis and/or multi-organ failure, comprising, identifying a patient as being at high risk for developing the vascular complications according to the above method (e.g., by measuring presence or levels of GOF or LOF mutations in the complement proteins in the patient sample, which indicates that the patient is high risk); and administering a complement modulator to the high risk patient.

Accordingly, embodiments of the present disclosure relate to use of inhibitors of the APC in preventing organ damage elicited by viral infection. In particular embodiments, the present disclosure provides use of inhibitors of the APC in reducing inflammatory response and severe organ damage in patients. Particularly, APC inhibitors (e.g., Compounds 1-4 and pharmaceutically acceptable salts thereof) are useful in preventing damage to lungs, heart, and kidneys in COVID-19 patients. In some embodiments, the inhibitors of APC are CFD inhibitors (e.g., one or more of Compounds 1-4 and pharmaceutically acceptable salts thereof). Exemplary CFD inhibitors are described in e.g., U.S. Pat. Nos. 9,598,446; 9,643,986; 9,663,543; 9,695,205; 9,732,103; 9,732,104; 9,758,537; 9,796,741; 9,828,396; 10,000,516; 10,011,612; 10,005,802; 10,081,645; 10,087,203; 10,092,584; 10,100,072; 10,106,563; 10,138,225; 10,189,869; 10,253,053; 10,287,301; 10,301,336; 10,370,394; 10,385,097; 10,428,095; 10,454,956; 10,550,140; 10,660,876; 10,662,175; 10,689,409; 10,807,952; 10,822,352; 10,906,887; 10,919,884, 11,084,800; International Publication No. WO 2020/041301, WO 2020/041301, and WO 2020/051532; and U.S. Patent Publication Nos. 2019-0382376, 2020-0062790, and 2020-0262818, the contents of which are incorporated herein by reference in their entirety.

In some embodiments, the inhibitors of APC are C5 inhibitors. Exemplary C5 inhibitors include, but are not limited to Eculizumab (Alexion); Ravulizumab (Alexion); Cemdisiran (Alnylam); LFG-316 (Novartis); SOB1005 (Sobi); SKY59 (RG6107/RO7112689 (Chugai and Roche)); REGN3918 (Regeneron); TNX-558 (Tanox); Neutrazumab (G2 Therapies); Coversin (Nomacopan) (Akari Therapeutics); CCX168 (Avacopan) (ChemoCentryx); ABP959 (Amgen); GNR-45 (Generium); Zimura (Ophthotech); RA101495 (Ra Pharma); ISU305 (ISU ABXIS); and Mubodina (Adienne).

In some embodiments, the inhibitors of APC are C5a inhibitors. Exemplary C5a inhibitors include, but are not limited to, IFX-1 (InflaRx) and Avacopan (CCX168 (Chemocentryx).

In some embodiments, the inhibitors of APC are C5aR1 inhibitors. Exemplary C5aR1 inhibitors include, but are not limited to, ALS-205 (Alsonex); DF2593A (Dompe); and IPH5401 (Innate Pharma).

In some embodiments, a C3 inhibitor can be used in conjunction with a C5 inhibitor. Exemplary C3 inhibitors include, but are not limited to, AMY-101 (Amyndas); APL-1 (Apellis); APL-2 (Apellis); and APL-9 (Apellis).

In some embodiments, Factor H can be used to treated activated APC in a subject, e.g., a COVID-19 patient.

The compositions and methods of the present disclosure can be used to assess whether a patient can benefit from a complement inhibitor. Examples of complement inhibitors generally include a protease inhibitor, a soluble complement regulator, a therapeutic antibody (monoclonal or polyclonal), complement component inhibitors, receptor agonists, or siRNAs.

Protease inhibitors include, but are not limited to, plasma-derived C1-INH concentrates, for example CETOR® (Sanquin), BERINERT® (CSL Behring, Lev Pharma), and CINRYZE®; and recombinant human C1-inhibitors, for example RHUCIN® and RUCONEST® (Pharming).

Soluble complement regulators include, but are not limited to, soluble complement receptor 1 (TP10) (Avant Immunotherapeutics); sCR1-sLeX/TP-20 (Avant Immunotherapeutics); MLN-2222/CAB-2 (Millenium Pharmaceuticals); and Mirococept (Inflazyme Pharmaceuticals).

Therapeutic antibodies include, but are not limited to, Eculizumab/Soliris (Alexion Pharmaceuticals); Pexelizumab (Alexion Pharmaceuticals); Ofatumumab (Genmab A/S); TNX-234 (Tanox); TNX-558 (Tanox); TA106 (Taligen Therapeutics); Neutrazumab (G2 Therapies); Anti-properdin (Novelmed Therapeutics); and HuMax-CD38 (Genmab A/S).

Complement component inhibitors include, but are not limited to, Compstatin/POT-4 (Potentia Pharmaceuticals); and ARC 1905 (Archemix).

Receptor agonists include, but are not limited to, PMX-53 (Peptech Ltd.); JPE-137 (Jerini); JSM-7717 (Jerini).

Others inhibitors include recombinant human MBL (rhMBL; Enzon Pharmaceuticals).

In further embodiments, complement inhibitors include, but are not limited to, OMS721 (OMS 00620646) (Omeros); Ravulizumab (ALXN1210) (Alexion); Coversin (Nomacopan) (Akari Therapeutics); CCX168 (Avacopan) (ChemoCentryx); IFX1 (CaCP29 (InfaRx); AMY-101 (Amyndas); APL-2 (Apellis); LNP023 (Novartis); Cemdisiran (ALN-CC5) (Alnylam); C11NH (Berinert) (CSL Behring); LFG-316 (Novartis).

Compositions and methods of the present disclosure can utilize inhibitors of the classical pathway of complement including, but not limited to, C1q inhibitors (ANX005, ANX007 (Annexon)); C1s inhibitors (BIW020 (Bioverativ)); C2 inhibitors (PRO-02 (Broteio/Argen-x)), as well as inhibitors of the lectin pathway including, but not limited to, MASP3 inhibitors (OMS906 (Omeros)).

In certain embodiments, a Factor B inhibitor can be used including, but not limited to, LNP203 (Novartis (Basel, Switzerland); Schubert et al., 116(16) PROC. NATL. ACAD. SCI. USA 7926-31 (2019)), anti-FB SiRNA (Alnylam Pharmaceuticals, Cambridge, Mass.); TA106 (monoclonal antibody, Alexion Pharmaceuticals, New Haven, Conn.); SOMAmers (aptamers, SomaLogic, Boulder, Colo.); bikaciomab (Novelmed Therapeutics, Cleveland, Ohio); complin (see, Kadam et al., J. Immunol. 2010, DOI: 10.4049/jimmunol.1000200); and Ionis-FB-LRx (ligand conjugated antisense drug, Ionis Pharmaceuticals, Carlsbad, Calif.)

Pharmaceutical Compositions and Formulations

The disclosure also relates to use of pharmaceutical compositions comprising at least one CFD inhibitor (e.g., at least one of Compound 1, Compound 2, Compound 3, and Compound 4; or a pharmaceutically acceptable salt thereof). Any suitable pharmaceutical compositions and formulations, as well as suitable methods for formulating and suitable routes and suitable sites of administration, are within the scope of this disclosure, and are known in the art. Also, unless otherwise stated, any suitable dosage(s) and frequency of administration are contemplated.

Unless otherwise noted, the dosage level of the at least one CFD inhibitor (e.g., at least one of Compound 1, Compound 2, Compound 3, and Compound 4; or a pharmaceutically acceptable salt thereof) can be any suitable level. In certain embodiments, the dosage levels of the at least one CFD inhibitor (e.g., at least one of Compound 1, Compound 2, Compound 3, and Compound 4; or a pharmaceutically acceptable salt thereof) for human subjects can generally be between about 1 mg/kg and about 100 mg/kg (e.g., between about 2 mg/kg and about 50 mg/kg, between about 5 mg/kg and about 25 mg/kg), per subject per treatment.

The plasma concentration in a subject, whether the highest level achieved or a level that is maintained, of the at least one CFD inhibitor (e.g., at least one of Compound 1, Compound 2, Compound 3, and Compound 4; or a pharmaceutically acceptable salt thereof) can be any desirable or suitable concentration. Such plasma concentration can be measured by methods known in the art. Such a plasma concentration of the at least one CFD inhibitor (e.g., at least one of Compound 1, Compound 2, Compound 3, and Compound 4; or a pharmaceutically acceptable salt thereof) in a subject can be the highest attained after administering the at least one CFD inhibitor (e.g., at least one of Compound 1, Compound 2, Compound 3, and Compound 4; or a pharmaceutically acceptable salt thereof) or can be a concentration of the at least one CFD inhibitor (e.g., at least one of Compound 1, Compound 2, Compound 3, and Compound 4; or a pharmaceutically acceptable salt thereof) in a subject that is maintained throughout the therapy. However, greater amounts (concentrations) may be required for extreme cases and smaller amounts may be sufficient for milder cases; and the amount can vary at different times during therapy.

In some embodiments, the at least one CFD inhibitor (e.g., at least one of Compound 1, Compound 2, Compound 3, and Compound 4; or a pharmaceutically acceptable salt thereof), is administered so that a C_{trough} of from about 100 ng/mL to about 600 ng/mL, particularly from about 150 ng/mL to about 300 ng/mL is maintained during treatment. In some embodiments, the at least one CFD inhibitor (e.g., at least one of Compound 1, Compound 2, Compound 3, and Compound 4; or a pharmaceutically acceptable salt thereof), is administered so that a minimum mean plasma concentration (C_{trough}) of at least 100 ng/mL, at least 150 ng/mL, at least 235 ng/mL, at least 300 ng/mL, or at least 600 ng/mL is maintained during treatment.

In some embodiments, the at least one CFD inhibitor (e.g., at least one of Compound 1, Compound 2, Compound 3, and Compound 4; or a pharmaceutically acceptable salt thereof), is administered so that a maximum plasma concentration (Cₘₐₓ) of less than about 1000 mg/mL, e.g., less than about 500 ng/mL, or less than 300 ng/mL (e.g., about 235 ng/mL) is achieved during treatment.

The at least one CFD inhibitor (e.g., at least one of Compound 1, Compound 2, Compound 3, and Compound 4; or a pharmaceutically acceptable salt thereof) can be administered to a subject as a monotherapy. In some embodiments, the methods described herein can include administering to the subject one or more additional treatments, such as one or more additional therapeutic agents.

The additional treatment can be any additional treatment, including experimental treatments, or a treatment for a symptom of an infectious disease, such as fever, etc. The other treatment can be any treatment, any therapeutic agent, that improves or stabilizes the subject's health. The additional therapeutic agent(s) includes IV fluids, such as water and/or saline, acetaminophen, heparin, one or more clotting factors, antibiotics, etc. The one or more additional therapeutic agents can be administered together with the at least one CFD inhibitor (e.g., at least one of Compound 1, Compound 2, Compound 3, and Compound 4; or a pharmaceutically acceptable salt thereof), as separate therapeutic compositions or one therapeutic composition can be formulated to include both: (i) one or more CFD inhibitors and (ii) one or more additional therapeutic agents. An additional therapeutic agent can be administered prior to, concurrently, or after administration of the one or more CFD inhibitors. An additional agent and the at least one CFD inhibitor (e.g., at least one of Compound 1, Compound 2, Compound 3, and Compound 4; or a pharmaceutically acceptable salt thereof) can be administered using the same delivery method or route or using a different delivery method or route. The additional therapeutic agent can be remdesivir.

In some embodiments, the additional therapeutic agent is a glycosaminoglycan such as heparan sulfate (HS), an α2,3 and α2,6 sialylated N-glycan, or an analog thereof, e.g., a synthetic analog of heparan such as RGTA® (ReGeneraTing Agent) or PI-88.

In some embodiments, the at least one CFD inhibitor (e.g., at least one of Compound 1, Compound 2, Compound 3, and Compound 4; or a pharmaceutically acceptable salt thereof) can be formulated with one or more additional active agents useful for treating a complement mediated disorder caused by a virus, e.g., a betacoronavirus such as SARS-CoV, MERS-CoV, and SARS-CoV-2, in a subject.

When the at least one CFD inhibitor (e.g., at least one of Compound 1, Compound 2, Compound 3, and Compound 4; or a pharmaceutically acceptable salt thereof) is to be used in combination with a second active agent, the agents can be formulated separately or together. For example, the respective pharmaceutical compositions can be mixed, e.g., just prior to administration, and administered together or can be administered separately, e.g., at the same or different times, by the same route or different route.

In some embodiments, a composition can be formulated to include a sub-therapeutic amount of the at least one CFD inhibitor (e.g., at least one of Compound 1, Compound 2, Compound 3, and Compound 4; or a pharmaceutically acceptable salt thereof) and a sub-therapeutic amount of one or more additional active agents such that the components in total are therapeutically effective for treating a complement mediated disorder caused by an infectious agent. Methods for determining a therapeutically effective dose of an agent are known in the art.

The compositions can be administered to a human subject using a variety of methods that depend, in part, on the route of administration. The route can be, e.g., oral, sublingual, buccal, transdermal, intradermal, intramuscular, parenteral, intravenous, intra-arterial, intracranial, subcutaneous, intraorbital, intraventricular, intraspinal, intraperitoneal, intranasal, inhalation, and topical administration.

In some embodiments, a composition is formulated for oral administration (“oral dosage forms”). Oral dosage forms can be, for example, in the form of tablets, capsules, a liquid solution or suspension, a powder, or liquid or solid crystals, which contain the active ingredient(s) in a mixture with non-toxic pharmaceutically acceptable excipients. These excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like. Compositions for oral administration may also be presented as chewable tablets, as hard gelatin capsules where the active ingredient is mixed with an inert solid diluent (e.g., potato starch, lactose, microcrystalline cellulose, calcium carbonate, calcium phosphate or kaolin), or as soft gelatin capsules where the active ingredient is mixed with water or an oil medium, for example, peanut oil, liquid paraffin, or olive oil. Powders, granulates, and pellets may be prepared using the ingredients mentioned above under tablets and capsules in a conventional manner using, e.g., a mixer, a fluid bed apparatus or a spray drying equipment.

Controlled release compositions for oral use may be constructed to release the active drug by controlling the dissolution and/or the diffusion of the active drug substance. Any of a number of strategies can be pursued in order to obtain controlled release and the targeted plasma concentration versus time profile. In one example, controlled release is obtained by appropriate selection of various formulation parameters and ingredients, including, e.g., various types of controlled release compositions and coatings. Examples include single or multiple unit tablet or capsule compositions, oil solutions, suspensions, emulsions, microcapsules, microspheres, nanoparticles, patches, and liposomes. In some embodiments, compositions include biodegradable, pH, and/or temperature-sensitive polymer coatings.

Dissolution or diffusion-controlled release can be achieved by appropriate coating of a tablet, capsule, pellet, or granulate formulation of compounds, or by incorporating the compound into an appropriate matrix. A controlled release coating may include one or more of the coating substances mentioned above and/or, e.g., shellac, beeswax, glycowax, castor wax, carnauba wax, stearyl alcohol, glyceryl monostearate, glyceryl distearate, glycerol palmitostearate, ethylcellulose, acrylic resins, dl-polylactic acid, cellulose acetate butyrate, polyvinyl chloride, polyvinyl acetate, vinyl pyrrolidone, polyethylene, polymethacrylate, methylmethacrylate, 2-hydroxymethacrylate, methacrylate hydrogels, 1,3 butylene glycol, ethylene glycol methacrylate, and/or polyethylene glycols. In a controlled release matrix formulation, the matrix material may also include, e.g., hydrated methylcellulose, carnauba wax and stearyl alcohol, carbopol 934, silicone, glyceryl tristearate, methyl acrylate-methyl methacrylate, polyvinyl chloride, polyethylene, and/or halogenated fluorocarbon.

The liquid forms in which compositions can be incorporated for administration orally include aqueous solutions, suitably flavored syrups, aqueous or oil suspensions, and flavored emulsions with edible oils, e.g., cottonseed oil, sesame oil, coconut oil, or peanut oil, as well as elixirs and similar pharmaceutical vehicles.

In some embodiments, the oral dosage form, such as a solution or suspension formed by mixing a triturated tablet or crystal or a powder with water, can be administered via a nasogastric tube.

A suitable dose of the at least one CFD inhibitor (e.g., at least one of Compound 1, Compound 2, Compound 3, and Compound 4, or a pharmaceutically acceptable salt thereof), which is capable of treating a complement mediated disorder caused by a virus, e.g., a betacoronavirus such as SARS-CoV, MERS-CoV, and SARS-CoV-2, in a subject, can depend on a variety of factors including, e.g., the age, gender, and weight of a subject to be treated and the particular inhibitor compound used. Other factors affecting the dose administered to the subject include, e.g., the type or severity of the complement mediated disorder caused by an infectious agent. Other factors can include, e.g., other medical disorders concurrently or previously affecting the subject, the general health of the subject, the genetic disposition of the subject, diet, time of administration, rate of excretion, drug combination, and any other additional therapeutics that are administered to the subject. It should also be understood that a specific dosage and treatment regimen for any particular subject will depend upon the judgment of the treating medical practitioner (e.g., doctor or nurse). A pharmaceutical composition can include a therapeutically effective amount of a CFD inhibitor. Such effective amounts can be readily determined by one of ordinary skill in the art.

In certain embodiments, the dosing of the at least one CFD inhibitor (e.g., at least one of Compound 1, Compound 2, Compound 3, and Compound 4; or a pharmaceutically acceptable salt thereof) can include administering to a human subject a loading dose of about 200 mg to about 500 mg of the at least one CFD inhibitor (e.g., at least one of Compound 1, Compound 2, Compound 3, and Compound 4; or a pharmaceutically acceptable salt thereof) on Day 1, followed by maintenance dose of about 600 mg to about 1200 mg (e.g., about 800 mg to about 1000 mg) administered in one or more doses per day, e.g., 150 mg to about 300 mg QID (e.g., about 200 mg to about 250 mg QID, about 200 mg QID, or about 250 mg QID) for the remainder of a treatment period (e.g., 14 days). After an initial treatment period, the at least one CFD inhibitor may be tapered, e.g., about 150 mg to about 300 mg TID (e.g., about 200 mg to about 250 mg TID, about 200 mg TID, or about 250 mg TID) for 2 days, followed by about 150 mg to about 300 mg BID (e.g., about 200 mg to about 250 mg BID, about 200 mg BID, or about 250 mg BID) for 2 days. The treating medical practitioner (such as a physician) can adjust the duration of treatment with the at least one inhibitor (e.g., at least one of Compound 1, Compound 2, Compound 3, and Compound 4; or a pharmaceutically acceptable salt thereof) and/or optionally request (or administer) additional treatment as needed. The subject can then be observed for 28 weeks following treatment with the at least one CFD inhibitor (e.g., at least one of Compound 1, Compound 2, Compound 3, and Compound 4; or a pharmaceutically acceptable salt thereof).

In some embodiments, a composition described herein contains a therapeutically effective amount of at least one CFD inhibitor (e.g., at least one of Compound 1 and Compound 2, or a pharmaceutically acceptable salt thereof), and one or more (e.g., one, two, three, four, five, six, seven, eight, nine, ten, or eleven or more) additional therapeutic agents to treat or prevent a complement mediated disorder caused by an infectious agent, such that the composition as a whole is therapeutically effective. For example, a composition can contain at least one CFD inhibitor (e.g., at least one of Compound 1, Compound 2, Compound 3, and Compound 4; or a pharmaceutically acceptable salt thereof) and an immunosuppressive agent, wherein the CFD inhibitor and agent are each at a concentration that when combined are therapeutically effective for treating or preventing a complement mediated disorder caused by virus in a human subject.

Methods for Screening for Test Agents

The present disclosure further provides methods for screening for test agents that are potentially useful in the treatment of COVID-19 and more specifically, screening test agents that are useful in treating severe vascular complications of COVID-19, such as, disseminated thrombosis and/or multi-organ failure, The method utilizes, inter alia, specialized cells, e.g., GPI-AP deficient cells and recognition of the phenomena that spike proteins (e.g., S1 and S2) from SARS-CoV-2, but not from unrelated coronaviruses such as benign coronavirus OC43, induce APC at the cell surface and promote cell death, which phenomena can be blocked by complement modulators such as (a) FD inhibitors; (b) C5 inhibitors; (c) C3 inhibitors; (d) a combination of C3 inhibitor and C5 inhibitor; and/or (e) FH protein.

Accordingly, in one embodiment, the disclosure relates to methods for screening for test agents that inhibit SARS-CoV-2 spike protein-mediated cell death, which includes, (a) incubating glycosylphosphatidylinositol-anchored protein (GPI-AP) deficient cells with human serum comprising recombinant SARS-CoV-2 spike proteins S1 and/or S2 for a period sufficient to induce alternate pathway of complement (APC) on the GPI-AP cells; (b) treating the incubate of (a) with a test agent (test incubate) or without a test agent (null incubate) and optionally one or more control agents (control incubate); (c) measuring cell viability of GPI-AP deficient cells in the test and null incubate of (b); (d) comparing the cell viability of GPI-AP deficient cells in the test incubate to the cell viability in the null incubate, and (e) optionally comparing to the cell viability in test incubate to the cell viability in the control incubate, wherein an increase in the cell viability in the test incubate compared to the cell viability in the null incubate indicates that the test agent is capable of inhibiting SARS-CoV-2 mediated cell death. In some embodiments, the control agent comprises an agent which effectively blocks the binding of SARS-CoV-2 spike proteins to cell surface, e.g., heparan sulfate (HS) or an analog or derivative thereof. HS derivatives include low molecular weight heparan sulfate (LMW-HS) and heparan analogs include mimetics such as OTR4120. See, Skidmore et al. J Med Chem. 2008; 51:1453-1458 26; Tong et al., Wound Repair & Regen., 17, 840-52, 2008; Tong et al., Wound Repair & Regen., 19, 505-14, 2011.

In some embodiments of the present disclosure, the GPI-AP deficient cell used in screening comprises a PIGA null blood cell, e.g., PIGAnull lymphoblast TF1 cells (TF1PIGAnull cells).

In embodiments, the screening methods may further include the step of determining deposition of complement proteins C5b9, C4d, and/or C3c on the surface of GPI-AP deficient cells in null and test samples and optionally in the control sample. In such embodiments, wherein complement deposition is additionally used as a parameter for assessment of test agents, a reduction in complement deposition in the presence of the test agent compared to that of a control or reference standard is indicative that the test agent is useful in the treatment of COVID-19.

In some embodiments, the screening methods are carried out in high throughput format, for example, wherein cell viability and/or deposition of complement proteins is measured using a fluorescence assay, e.g., fluorescence activated cell sorter (FACS).

A test agent can be selected from a number of different modalities. A test agent can be an antibody, a nucleic acid molecule (e.g., DNA molecule or RNA molecule, e.g., mRNA or inhibitory RNA molecule (e.g., short interfering RNA (siRNA), microRNA (miRNA), or short hairpin RNA (shRNA)), or a hybrid DNA-RNA molecule), a peptide, a small molecule, an inhibitor of a signaling cascade, an activator of a signaling cascade, or an epigenetic modifier), or an aptamer. Any of these modalities can be a complement inhibitor directed to target (e.g., to inhibit) function of a complement protein; complement expression; complement binding; or complement signaling. The nucleic acid molecule or small molecule may include a modification. For example, the modification can be a chemical modification, e.g., conjugation to a marker, e.g., fluorescent marker or a radioactive marker. The modification can also include conjugation to an antibody to target the agent to a particular cell or tissue. Additionally, the modification can be a chemical modification, packaging modification (e.g., packaging within a nanoparticle or microparticle), or targeting modification.

The following examples are merely illustrative and should not be construed as limiting the scope of this disclosure in any way as many variations and equivalents will become apparent to those skilled in the art upon reading the present disclosure.

### EXAMPLES

### Example 1: Blocking the Direct Activation of the Alternative Complement Pathway by SARS-CoV-2 Spike Protein with a Complement Factor D Inhibitor

A study was performed to show that the SARS-CoV-2 spike protein subunits are potent activators of the APC and that Compound 2 prevents the complement-mediated damage.

Materials and Methods

Human Coronavirus Proteins

Recombinant proteins expressed with E. Coli system: SARS-CoV-2 S1 subunit protein (RBD) (S1, Cat. 230-01101, RayBiotech), S2 subunit (S2, Cat. 230-01103, RayBiotech) and nucleocapsid protein (N, Cat. RP01264, ABclonal Technology). Recombinant proteins expressed with baculovirus-insect cell system: Human coronavirus spike protein (HCoV-OC43 S, Cat. 40607-V08B, Sino Biological). Coronavirus proteins were used to activate complement by adding into normal human serum (NHS, Cat. NHS Complement Technology, Inc.). Heat denaturation of human coronavirus proteins was performed by heating proteins at 100° C. for 30 minutes.

Modified Ham Test

The mHam assay was used to test complement activation in serum as described in Gavriilaki et al. Blood. 2015; 125(23):3637-3646. i). Cell preparation: TF1PIGAnull cells were maintained at a density of 500,000 cells/mL daily. Before the assay, the cells were washed with phosphate-buffered saline (PBS) and seeded in a round-bottom 96-well plate with a density of 6,700 cells/well in 80 μL GVB⁺⁺ buffer (Cat. B102, Complement Technology, Inc) in triplicate. ii). Serum Preparation: 20 μL NHS (Cat. B102, Complement Technology, Inc) was added with 0.25, 0.5, 1.0 and 2.0 μg human coronavirus proteins (final concentration of S1, S2, N, and HCoV-OC43 S are 2.5, 5, 10 and 20 μg/mL) and incubated on ice for 15 minutes. As a negative control, NHS was heated at 56° C. for 30 min to inactivate NHS complement activity (NHS (H)). For the complement rescuing mHam, NHS was first incubated with Compound 2 diluted in dimethyl sulfoxide (DMSO) (final concentration 1.0 μM) or 50 μg anti-C5 antibody (anti-C5Ab, Alexion pharmaceuticals) on ice for 15 min, and then added with SARS-CoV-2 spike proteins for another 15 i5 min on ice. iii) Complement reaction: the 20 μL serum mixture was added to 80 μL cells and incubated at 37° C. for 45 min with constant shaking. After incubation, cells were separated by centrifugation at 600 g for 3 min at room temperature and washed with PBS. iv) Cell viability assay: After washing, the cells in each well were resuspended in 100 μL 10% WST-1 proliferation solution (WST-1: RPMI 1640 without phenol red at a ratio of 1:9, WST-1 Cat. 11644807001, Roche, Switzerland) and incubated for 2 hours at 37° C. WST-1 solution was used as a blank control. The absorbance of the chromogenic metabolized product was measured with a plate reader (ELX808, BioTeK, Winooski, VT) at 450 nm with a reference wavelength at 630 nm. v) Percentage of Non-viable cells calculation: the sample absorbance value was normalized by subtracting the absorbance of a blank control. The percentage of live cells was calculated as the ratio of normalized sample absorbance (A_{450-630nM}) to normalized negative control NHS(H) absorbance multiplied by 100 (Formula: % live cells=[(sample A_{450-630nM}−blank A_{450-630nM})/(NHS (H) A_{450-630nM}−blank A_{450-630nM})×100]). Complement activation level was indicated by the percentage of non-viable cells (100−% live cells). Based on a receiver operative curve, Z 20% non-viable cells (cell killing) have been established as a positive test (Vaught et al., JCI Insight. 2018; 3(6)).

Detection of Complement Activity by Flow Cytometry

Cell surface depositions of C5b-9, C3c, and C4d on TF1PIGAnull cells were measured by flow cytometry. i) Cell preparation: Before the assay, TF1PIGAnull cells were washed with PBS and seeded in a in V-bottom 96-well plates (1.2×10⁵ cells/well) in 80 μL of either GVB⁺⁺ buffer or GVB⁰·10 mM MgEGTA buffer (pH 6.4) (GVB⁰ Cat. B103, Complement Technology, Inc). GVB⁺⁺ allows all complement pathways activation while GVB⁰·MgEGTA only allows alternative pathway activation. ii) Serum preparation: 20 μL NHS was added with 0.25, 0.5, 1.0 and 2.0 μg human coronavirus proteins (final concentration of S1, S2, N, and HCoV-OC43 S ranges from 2.5 to 20 μg/mL) and incubated on ice for 15 minutes. For alternative pathway activation, NHS was acidified to pH 6.4 by adding 0.2 M HCl. iii) Complement reaction: The 80 μL cells were incubated with the 20 μL serum mixture for 15 min at 37° C. with constant shaking, and the reaction was stopped by adding FACS buffer (PBS supplemented with 1% BSA and 15 mM EDTA). Cells were centrifuged at 600 g for 3 min at room temperature and washed with PBS. NHS with 5 mM of ethylenediaminetetraacetic acid (EDTA), which inhibits complement activation, was used as a negative control. As a positive control for C4d detection, 10 μg/mL Shiga toxin 1 (Cat. SML0562, Sigma-Aldrich, St. Louis, MO) was incubated with NHS on ice for 15 min, followed by the addition of cells in GVB⁺⁺ buffer. Complement inhibitors, Compound 2 (final concentration 1.0 μM) and anti-C5Ab (50 μg/sample) were also used to identify the specific complement pathway(s) involved. iv) Staining and detecting: Cells were washed with PBS and stained with anti-C5b-9 monoclonal antibody (Cat. sc-58935, Santa Cruz Biotechnology, Inc., dilution at 1:100) for 30 min on ice. Then, cells were washed with PBS and stained with Alexa 647 conjugated secondary antibody (Cat. Ab172325, Abcam, dilution at 1:500) and Alexa 488 conjugated anti-C3c antibody (Cat. 4212, Abcam, dilution at 1:150) for another 30 min on ice. The cells were also labeled with anti-C4d biotinylated monoclonal antibody (Cat. A704, Quidel, dilution at 1:50) and PE-Streptavidin (Cat. 554061, BD Pharmingen, dilution 1:500). Ten thousand events per sample were collected by a BD FACSCalibur and data was analyzed using FlowJo software version 10.5.3 (FlowJo Inc).

Quantification of Serum Factor Bb by ELISA

Serum Bb level was measured by MicroVue Bb Plus EIA kit (Cat. A027, Quidel). To determine the increase of serum Bb concentration in the presence of cells, 20 μL NHS was preincubated with 20 μg/mL SARS-CoV-2 spike proteins for 15 min on ice, followed by the addition of 80 μL of either GVB⁰·MgEGTA buffer (pH 6.4) or TF1PIGAnull cells (1.2×10⁵ cells/sample) in GVB⁰·MgEGTA buffer (pH 6.4). After the reaction was incubated for 15 min at 37° C., the cells were centrifuged at 600 g for 3 min at room temperature and the supernatant was collected for Bb quantification. The assay was also performed with Compound 2 (1.0 μM) and anti-C5Ab (50 μg/sample).

Flow Cytometry Assay for SARS-CoV-2 Spike Proteins Binding to TF1PIGAnull Cells and Blockade with Heparan Sulfate

Flow cytometry was performed to evaluate the binding of SARS-CoV-2 spike proteins onto the cell surface. Heparan sulfate sodium salt (NaHS, Cat. H7640, Sigma-Aldrich) was dissolved in PBS with 0.5% BSA to reach a concentration of 2 mg/mL. TF1PIGAnull cells (50,000 cells/sample) was washed and resuspended in either 100 μL PBS with 0.5% BSA or the NaHS/PBS solution with 0.5% BSA. The cells were then added with 5 μg/mL S1 or S2 with C terminal His-tag and incubated for 15 min at 37° C. After incubation, the cells were washed and fixed with 4% formaldehyde solution for 15 min at room temperature. To measure the binding of His-tagged S1 and S2 to the cell surface, the cells were stained with anti-His-tag antibody (Cat. sc-53073, Santa Cruz Biotechnology, Inc., dilution at 1:100) for 30 min on ice, followed by Alexa 488 goat anti-mouse IgG (Cat. A11001, Invitrogen, dilution at 1:200) for 10 min on ice in the dark. His-tagged S1 and S2 binding to cells was measured by a BD FACSCalibur.

Complement Inhibition with Purified Factor H Protein

10 μg purified factor H proteins (Cat. A137, Complement Technology, Inc.) and 20 μg/mL S1 were added to 20 μL NHS and incubated for 15 min at 37° C. TF1PIGAnull cells (120,000 cells/sample) in 80 μL GVB0·MgEGTA buffer (pH 6.4) were then added to the serum mixture followed by incubation for 15 min at 37° C. with constant shaking. After the reaction, the cells were washed and stained with an anti-C5b-9 monoclonal antibody (Cat. sc-58935, Santa Cruz Biotechnology, Inc., dilution at 1:100) on ice for 30 min followed by Alexa 647 conjugated secondary antibody (Cat. Ab172325, Abcam, dilution at 1:500). Cells were also stained with Alexa 488 conjugated anti-C3c antibody (Cat. 4212, Abcam, dilution at 1:150). C5b-9 and C3c depositions on the cell surface were measured by a BD FACSCalibur.

Data Analysis

All experiments were performed at least three times. All data was summarized as mean±SD or SE, and Student's t-test was used to assess the difference between unpaired groups. A P-value less than 0.05 (P<0.05) was considered statistically significant (*).

Human Coronavirus Protein Binding to TF1PIGAnull Cells and Sialic Acid Removal by Sialidase

TF1PIGAnull cells were seeded in V-bottom 96-well plates (1.2×10⁵ cells/well) in 80 μL GVB0·10 mM MgEGTA buffer (pH 6.4) and added with 20 μg/mL His-tagged human coronavirus protein (S1, S2, N, and HCoV-OC S) for 15 min at 37° C. and fixed with 4% formaldehyde solution for 15 min at room temperature. Cells were centrifuged at 600 g for 3 min at room temperature and stained with anti-His-tag antibody (Cat. sc-53073, Santa Cruz Biotechnology, Inc., dilution at 1:100) followed by Alexa 488 goat anti-mouse IgG (Cat. A11001, Invitrogen, dilution at 1:200) for 30 min on ice in the dark.

To evaluate the effect of sialic acid on the binding of S1 to TF1PIGAnull cells, cell surface sialic acid residues were removed by α2-3,6,8 sialidase (Sia, Cat. P0720L, New England Bio Labs). Cells (120,000 cells/sample) in 80 μL GVB0·10 mM MgEGTA buffer (pH 6.4) were first incubated with 50 Units/mL Sia for 20 min at 37° C. with constant shaking, before added with 20 μg/mL His-tagged S1. Cells were also stained with biotinylated MALII (Cat. B-1265, Vector Laboratories, dilution at 1:50) followed by PE-Streptavidin (Cat. 554061, BD Pharmingen, dilution 1:500) to confirm sialic acid removal.

Results

SARS-CoV-2 Spike Proteins (S1 and S2) Induce Cell Killing Through the APC

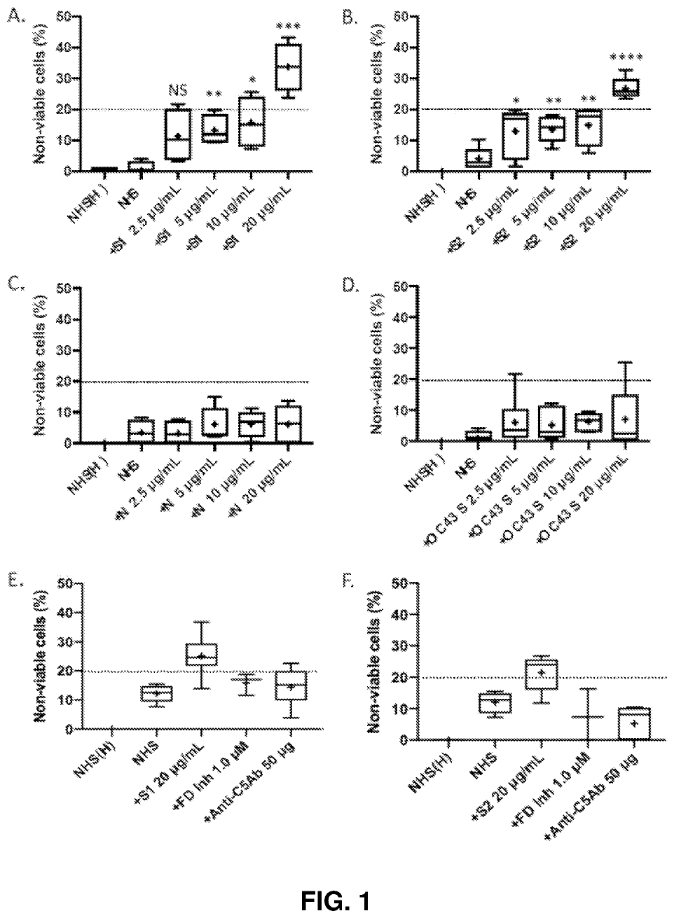

The ability of spike proteins to activate complement was first tested via a cell-based mHam assay that has been previously validated for detecting complement-driven diseases such as aHUS, CAPS and the HELLP syndrome (Chaturvedi, 2020, supra; Gavriilaki, 2015, supra; and Vaught, 2018, supra). Spike protein S1 and S2 subunits from SARS-CoV-2 added to normal human serum induced dose-dependent cell killing in the mHam assay (FIGS. 1A-B), which was inhibited by Compound 2 and anti-C5Ab (FIGS. 1E-F). The SARS-CoV-2 N protein and the spike protein from the benign human coronavirus OC43 did not increase killing in the mHam (FIGS. 1C-D). Since factor D is specific to the APC, these data suggest that S1 and S2 subunits of SARS-CoV-2 spike protein activate complement primarily through the alternative pathway.

SARS-CoV-2 Spike Proteins (S1 and S2) Increase APC Markers on Cells

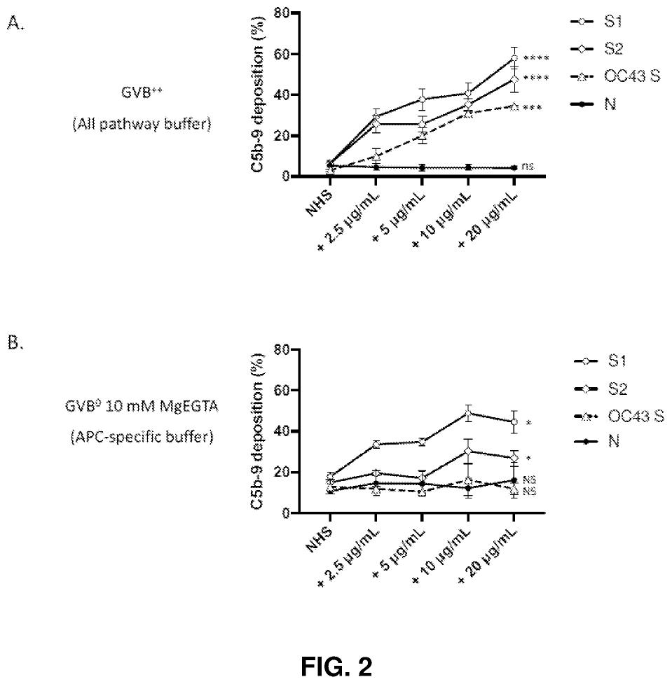

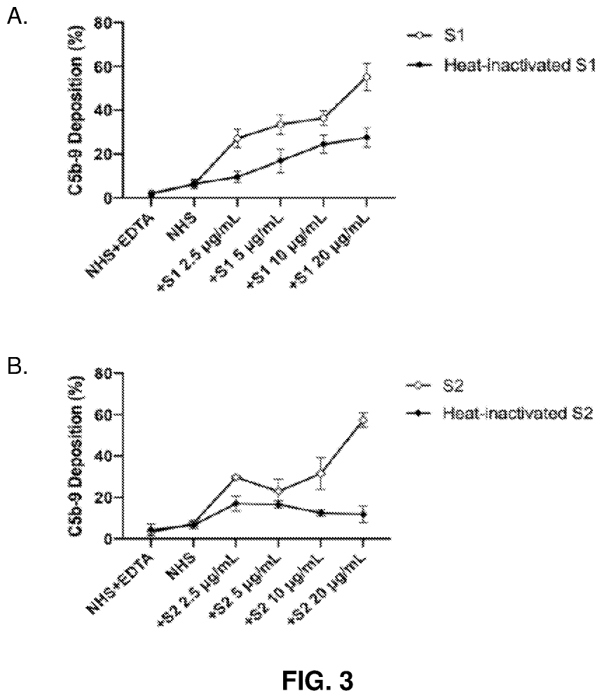

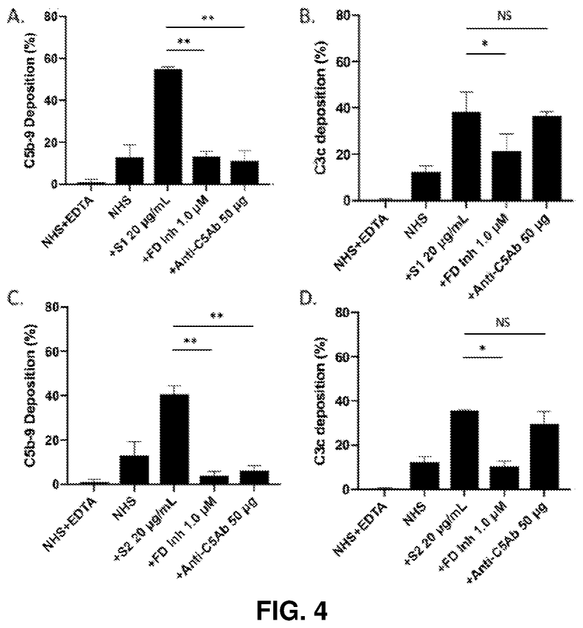

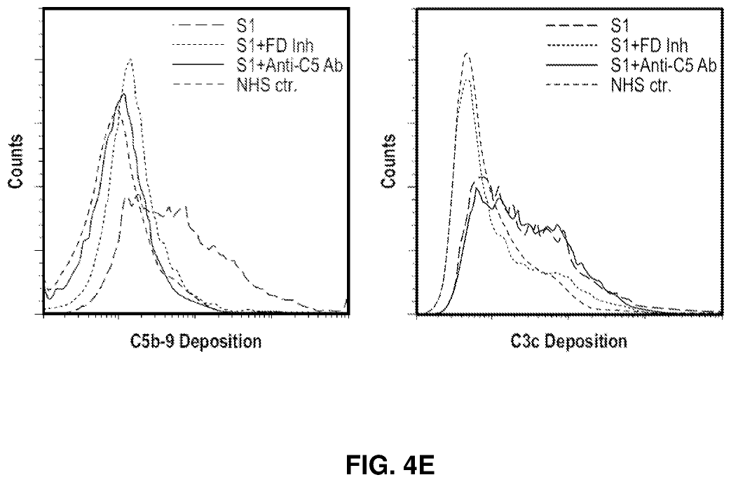

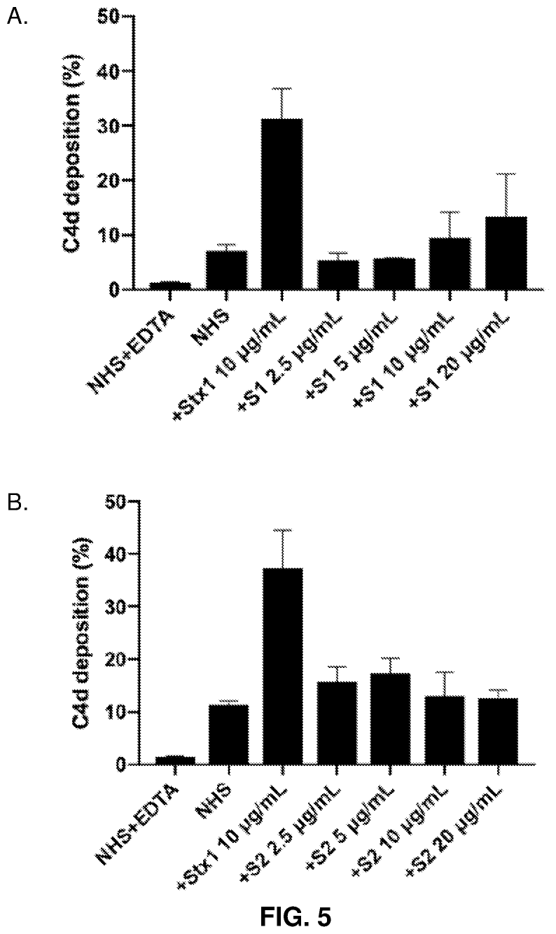

Biomarkers of complement activation, C3c, C4d, and C5b-9 deposition, were detected with flow cytometry. S1 and S2 subunits from SARS-CoV-2 added to normal human serum increased C5b-9 deposition in a dose-dependent manner that correlated with complement-dependent killing in the mHam (FIGS. 2A and 2B). Denaturing the S1 and S2 proteins with heat before adding to normal human serum prevented the accumulation of C5b-9 deposition on cell surface (FIGS. 3A and 3B). C5b-9 deposition induced by S1 and S2 in both GVB++ and GVB0·MgEGTA buffer were inhibited by anti-C5Ab and Compound 2; however, Compound 2 was more potent in blocking C3c deposition since it targets the upstream cascade of the APC, especially in GVB0·MgEGTA (FIGS. 4A-4E). C4d deposition was modestly increased in response to S1 and S2 (FIG. 5A and FIG. 5B, respectively). Taken together, the data confirms that S1 and S2 subunits of SARS-CoV-2 spike protein mainly activate the alternative complement pathway.

SARS-CoV-2 S1 Spike Protein Requires the Cell Surface to Activate the APC

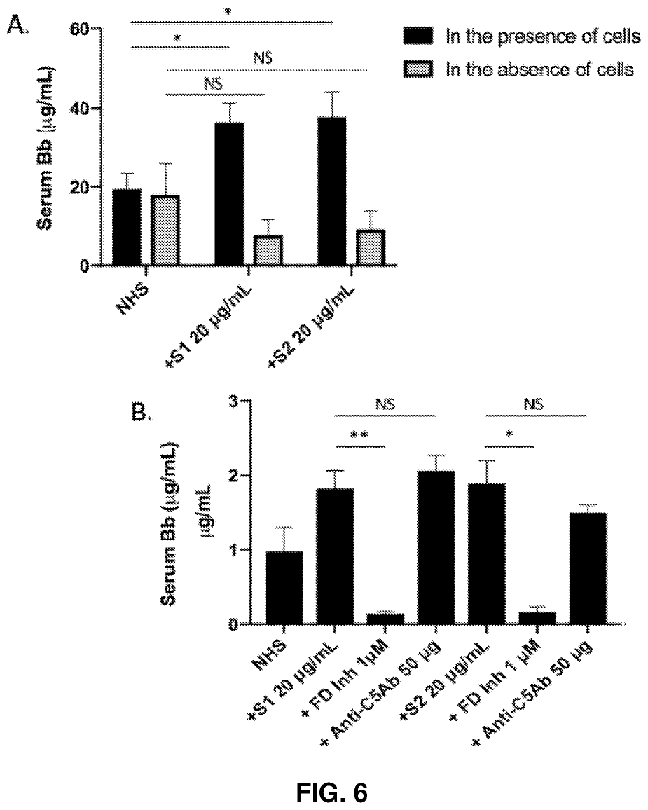

Next, a test was performed to determine whether the activation of the APC induced by spike proteins was in fluid phase or on cell surfaces. The results are shown in FIGS. 6A and 6B. Serum level of complement factor Bb, a specific marker of AP activation, was elevated in the supernatant from TF1PIGAnull cells incubated with S1 and S2 (FIG. BA), which the elevation of serum Bb concentration was inhibited by Compound 2 but not anti-C5Ab (FIG. 6B). The addition of S1 or S2 to human serum in the absence of TF1PIGAnull cells did not result in elevated Bb levels, which demonstrates that the spike proteins activate the APC predominantly on the cell surface and not in the fluid phase.

SARS-CoV-2 S1 and S2 Spike Protein Bind to the Heparan Sulfate on the Cell Surface

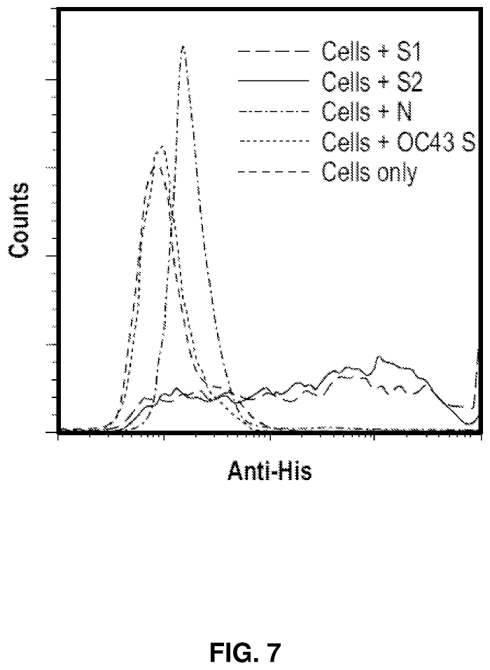

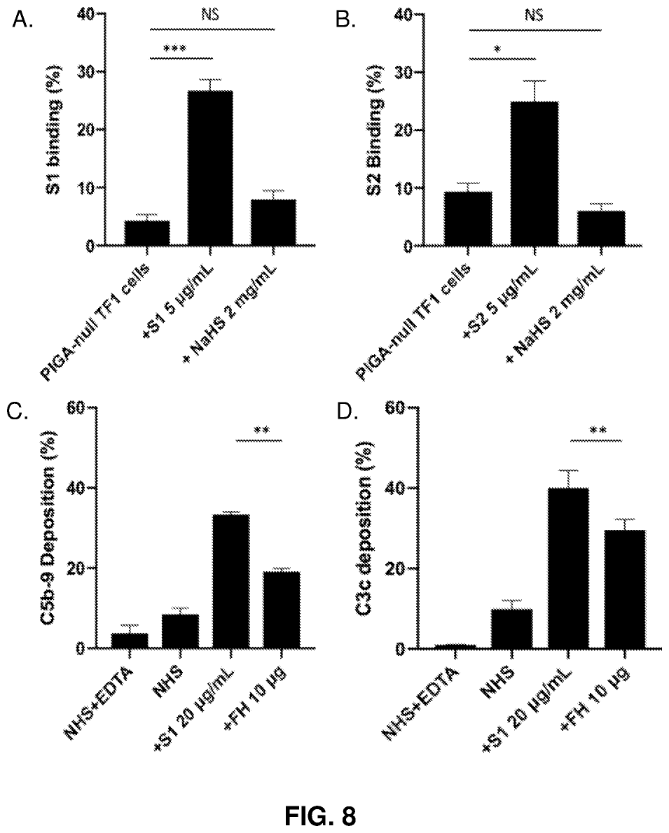

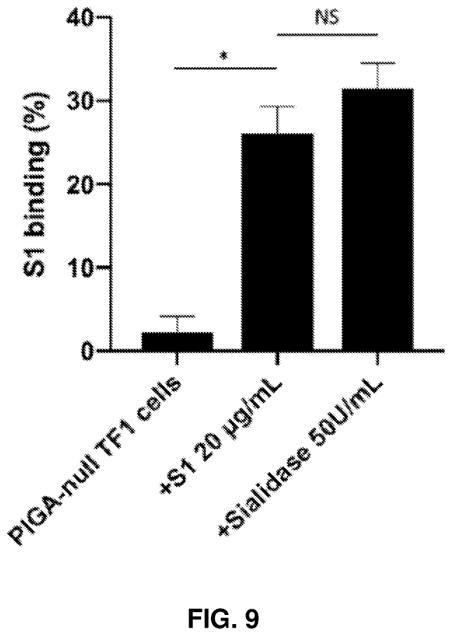

As an erythroblast cell line, TF1PIGAnull expresses little ACE2, the entry receptor for SARS-CoV-2, which makes it a good model for the study of spike protein-glycosaminoglycans interaction (Li et at, Infectious Diseases of Poverty. 2020; 9(1):45). Several groups have proved that SARS-CoV-2's spike protein binds to heparan sulfate (HS) but not α2,3 or α2,6 sialic acids by microarray (Hao et al., bioRxiv. 2020:2020.2005.2017.100537; Liu et al., bioRxiv. 2020:2020.2005.2010.087288). Additional research showed that there are three heparan sulfate binding sites on the spike protein: one located inside the receptor binding domain (RBD) of S1, one located in S2, and another one located at the S1/S2 cleavage sites (Kim et al., Antiviral Res. 2020:104873). Soluble HS has been shown to effectively block the binding of viruses that utilize HS on cell surfaces, therefore the present inventors hypothesized that this would also be true for SARS-CoV-2 (Viasak et al., Journal of Virology. 2005; 79(1):5963-5970). Both S1 and S2, but not HCov-OC43 bind TF1PIGAnull cells (FIG. 7), however S1 and S2 binding was significantly blocked by free HS (FIG. 8A-8B). Treatment with sialidase, which removes all α2-3, α2-6, and α2-8 linked sialic acid residues, did not result in any decrease in spike protein binding (FIG. 9). These results confirm that HS is required for spike protein binding to cell surfaces and are consistent with recently published microarray data (Hao et al, 2020, supra; Liu et al., 2020, supra).

Factor H Decreases C3c and C5b-9 Deposition Caused by S1 Spike Proteins

Apart from virus interaction, heparan sulfate on cell surfaces is also important for binding factor H, a negative regulator of the APC (Jokiranta et al., Am J Pathol. 2005; 167(4):1173-1181; Hyvarinen et al., Blood. 2016; 127(22):2701-2710). S1 subunit of SARS-CoV-2 is of particular interest because S1 will be cleaved and released upon infection, while S2 will be inserted into the host cell membrane. Thus, the present inventors hypothesized that S1 may be disrupting factor H binding or activity on the cell surface.

As shown in the data presented herein, addition of purified factor H protein to normal human serum incubated with S1 decreased C5b-9 and C3c deposition on TF1PIGAnull cells (FIGS. 8C and 8D). Taken together, these data demonstrate that the S1 subunit binds to heparan sulfate and appears to interfere with factor H function.

Conclusion

COVID-19 is a life-threatening infectious disease that often results in hypercoagulability, thrombotic microangiopathy, and severe endothelial damage (Gavriilaki, 2020, supra).; Spiezie et al., Thromb Haemost. 2020; 120(6):998-1000; Marchetti., Ann Hematol. 2020; 99(8):1701-1707). The role of complement activation and its contribution to disease severity is increasingly recognized, but the mechanism of complement activation was unknown (Java et al., JCI Insight. 2020; 5(15): e140711). Here, it was demonstrated that SARS-CoV-2 spike protein, but not spike protein from a benign human coronavirus HCoV-OC43 or the SARS-CoV-2 N protein, activates the APC on cell surfaces and leading to deposition of complement activators such as C5b9 and C3c. Both S1 and S2 subunits activate the APC, but only in the presence of cells, demonstrating that APC activation is occurring on the cell surface and not the fluid phase. Structure resolution of the receptor binding domain (RBD) in SARS-CoV-2 spike protein S1 subunit show a small positively charged region that may bind to negatively charged heparan sulfate (Lan et al., Nature. 2020; 581(7807(:215-220). The present findings confirm that both spike protein S1 and S2 subunits bind to heparan sulfate, but counter previous data that SARS-CoV-2 N proteins activate the lectin pathway (Gao, 2020, supra). Soluble heparan sulfate, but not sialidase treatment of target cells, blocked spike proteins from binding target cells.

The highly conserved nature of spike protein S1 and S2 among different coronaviruses (particularly among the identified strains of SARS-CoV-2), indicates a role of spike proteins in coronaviral disease pathology. Here, it was shown that Compound 2, complement factor D inhibitor, was able to block the APC activation triggered by SARS-CoV-2 spike proteins and inhibit deposition of complement proteins C5b9 and C3c on the surface of cells. More importantly, Compound 2 prevented cell death elicited by APC activation in an established cellular model of coronaviral disease. In addition, C5Ab antibody also blocks mHam killing and C5b-9 deposition but does not block deposition of C3 fragments or the release of Bb into serum supporting complement activation primarily through the APC. These data demonstrate the usefulness of CFD inhibitors such as Compounds 1 and 2, in the treatment and management of coronaviral diseases such as COVID19.

Factor H, a negative regulator of the APC, achieves a more active conformation when it binds to cells through interactions with glycosaminoglycans (heparan sulfate and α2-3 N-linked sialic acid residues) (Hyvarinen et al., Blood. 2016; 127(22):2701-1710; Hebert et al., J Immunol. 2015; 195(10):4986-4998; Langford-Smith et al., Front Immunol. 2015; 6:25) Factor H is made up of 20 CCP modules (also referred to as sushi domains or short consensus repeats). CCP1-4 bind to C3b, CCP6-8 bind heparan sulfate, and CCP 19-20 bind heparan sulfate and sialic acids (Perkins et al., Front Immunol. 2014; 5(126); Osbourne et al., Journal of Biological Chemistry. 2018; 293(44):17166-17187). Several human diseases, such as aHUS, age-related macular degeneration (AMD), HELLP syndrome, and CAPS, are associated with genetic variants that affect factor H function (Chaturvedi, 2020, supra; Rodriguez De Córdoba et al., seminars in Thrombosis and Hemostasis. 2014; 40(4):422-430; Vaught et al., J Matern Fetal Neonatal Med. 2020:1-9). Shiga toxin from enterohemorrhagic E. coli also leads to endothelial damage and has been shown to bind to complement control protein (CCP) 6-8 or CCP 18-20 of factor H and impair regulation of the APC on cell surfaces (Orth et al., J Immunol. 2009; 182(1):6394-6400). Germline variants affecting CCP 19 and 20 in factor H predispose to aHUS and polymorphism in CCP 7 is found in up to 35% of patients with AMD (Raychaudhuri et al., Nat Genet. 2011; 43(12):1232-1236; Fritsche et al., Annual Review of Genomics and Human Genetics. Vol. 15: Annual Reviews Inc.; 2014:151-171). Interestingly, a history of AMD was recently reported to be a leading risk factor for intubation and death in COVID-19 patients (Ramiall et al., medRxiv. 2020).

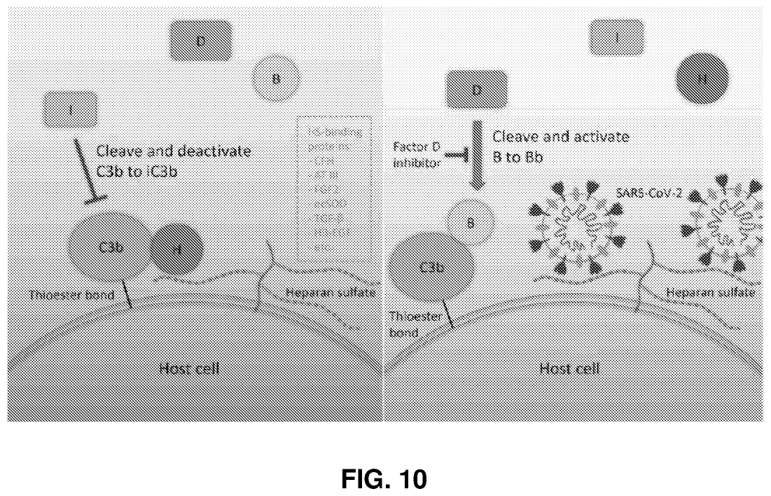

The above-described binding assays (FIG. 8A-8D) suggest that SARS-CoV-2 spike proteins bind cell surface heparan sulfate, preventing factor H from achieving its optimal conformation to accelerate decay of the APC C3 convertase (FIG. 10). Further, rescue experiments also showed that addition of purified factor H to serum decreased C3c and C5b-9 deposition on spike protein treated cells, suggesting factor H a key player in the APC activation by the spike protein. Whether spike protein directly blocks factor H from binding to HS or indirectly affect the conformation of factor H, however, remains to be further explored.

That SARS-CoV-2 spike proteins activate the APC has profound implications for understanding the multi-organ dysfunction, coagulopathy, and endothelial injury characteristic of COVID-19. Increased levels of C5a and soluble C5b-9 are detected in patients with moderate to severe COVID-19 (Cugno et al., J Allergy Clin Immunol. 2020). Patients with COVID-19 also develop renal failure and some have biopsy-proven thrombotic microangiopathies (Jhaveri et al., Kidney Int. 2020; Hirsch et al., Kidney Int. 2020). Thrombosis that is only partially responsive to anticoagulation (resistance to heparin treatment and thrombosis that develops despite appropriate prophylactic anticoagulation) is common in COVID-19 and characteristic of complementopathies (Markiewski et al., Trends in Immunology. 2007; 28(4):184-192; Conway et al., Blood Coagul Fibrinolysis. 2018; 29(3):243-251), such as paroxysmal nocturnal hemoglobinuria (Hill et al., Blood. 2007; 110(12):4123-4128), cold-agglutinin disease (Baines et al., Blood Rev. 2017; 31(4):213-223), and CAPS (Chaturvedi, 2020, supra). In addition, heparan sulfate is a binding partner for antithrombin Ill which could further increase hypercoagulability in COVID-19 and may explain the heparin resistance that is frequently encountered in these patients (Langdown et al., J Mol Biol. 2009; 386(5):1278-1289. Heparan sulfate also interacts with many extracellular proteins, including fibroblast growth factor 2 (FGF2), vascular endothelial growth factor (VEGF), transforming growth factor β (TGF-0), heparin-binding epidermal growth factor (HB-EGF), and extracellular superoxide dismutase (ecSOD) (Nugent et al., Biochemistry (Mosc). 2013; 78(7):726-735), suggesting a broad influence by the SARS-CoV-2 spike protein.

The data provided herein may explain why only a minority of COVID-19 patients develop life-threatening multi-organ failure. Complement-driven diseases such as the aHUS, HELLP and CAPS often have underlying germline variants that impair the ability of endothelial cells to protect themselves from complement-mediated injury in the setting of a complement amplifying condition such as pregnancy, cancer, autoimmunity, or other inflammatory states (Vaught, 2018, supra; Vaught et al., Exp Hematol. 2016; 44(5):390-398). A recent proteomics study by Bo Shen et al. with a machine learning model confirmed that complement proteins are actively involved in the acute phase of virus infection, including complement 6 (C6) and complement factor B (CFB), Properdin (CFP), and Carboxypeptidase N catalytic chain (CPN1) (Shen et al., Cell. 2020; 182(1):59-72.e15). Thus, it will be important to determine whether patients with the most severe forms of COVID-19 harbor variants in complement regulatory genes. Already, a history of AMD, a disease associated with a common factor H variant, was identified as a major risk factor for mortality in COVID-19 (Ramlall, 2020, supra). The data provided herein also have therapeutic implications for COVID-19 and raise the prospect for targeted therapy. Multinational randomized controlled trials of the C5 monoclonal ravulizumab (phase 3, NCT04369469) are already enrolling patients; however, the data presented herein demonstrates that C5 inhibition will prevent C5b-9 accumulation but not upstream complement activation induced by the SARS-CoV-2 spike proteins.

Dense C3b deposition can lead to breakthrough from C5 monoclonal antibodies (Harder et al., Blood. 2017; 129(8):970-980; Brodsky et al., Haematologica. 2020). Thus, complement inhibitors that bind upstream of factor H may be more specific and effective (Yuan et al., Haematologica. 2017; 102(3):466-475; Risitano et al., Front Immunol. 2019; 10(1157)).

### Example 2: Big Effect Trial (BET) for the Treatment of COVID-19—Danicopan/Remdesivir vs. Placebo/Remdesivir

Study Design

This platform trial will conduct a series of randomized, double-blind, placebo-controlled trials using a common assessments and endpoints in hospitalized adults diagnosed with COVID-19. This is a proof-of-concept study with the intent of identifying promising treatments to enter a more definitive study (ACTT or other). The study will be conducted in up to 40 sites throughout the US. The study will compare different investigational therapeutic agents to a common control arm and determine which have relatively large effects. In order to maintain the double blind, each intervention will have a matched placebo. However, the control arm will be shared between interventions and may include participants receiving the matched placebo for a different intervention.

Subjects will be assessed daily while hospitalized (see Table 2). The schedule is similar to ACTT to allow evaluation of endpoints used in the larger study. Once subjects are discharged from the hospital, they will have a study visit at Days 15, 22, 29, and 60 as an outpatient. The Day 22 and Day 60 visits do not have laboratory tests or collection of samples and may be conducted by phone. If infection control and other limitations limit the ability to have visits at Days 15 and 29 in person, they may also be conducted by phone and only clinical data will be obtained.

All subjects will undergo a series of efficacy and safety laboratory assessments. Safety laboratory tests and blood (serum and plasma) research samples and oropharyngeal (OP) swabs will be obtained on Day 1 (prior to study product administration) and Days 3, 5, 8, and 11 while hospitalized. OP swabs (oropharyngeal swabs are preferred, but if these are not obtainable, saliva or nasopharyngeal or nasal swabs may be substituted) and blood (serum only) plus safety laboratory tests will be collected on Day 15 and 29 if the subject attends an in-person visit or is still hospitalized.

Study Objectives

The study objectives and corresponding end points are shown in Table 1.

TABLE 1
BET-Danicopan Study Objectives

| | ENDPOINTS |
|---|---|
| OBJECTIVES | (OUTCOME MEASURES) |
| Primary | |
| To evaluate the clinical efficacy of different | 1. Not hospitalized, no limitations on activities; |
| investigational therapeutics relative to the control | 2. Not hospitalized, but new or increased |
| arm in adults hospitalized with COVID-19 | limitation on activities and/or requiring new or |
| according to clinical status (8-point ordinal scale) | increased home oxygen, CPAP or BiPAP; |
| at Day 8. | 3. Hospitalized, not requiring supplemental |
| | oxygen - no longer requires ongoing medical |
| | care; |
| | 4. Hospitalized, not requiring supplemental |
| | oxygen - requiring ongoing medical care (COVID- |
| | 19 related or otherwise); |
| | 5. Hospitalized, requiring new or increased |
| | supplemental oxygen; |
| | 6. Hospitalized, requiring new or increased non- |
| | invasive ventilation or high-flow oxygen devices; |
| | 7. Hospitalized, on invasive mechanical |
| | ventilation or ECMO; |
| | 8. Death. |
| Key Secondary | |
| 1. To evaluate the clinical efficacy of different | Day of recovery is defined as the first day on |
| investigational therapeutics as assessed by time | which the subject satisfies one of the following |
| to recovery compared to the control arm. | three categories from the ordinal scale: |
| | 1. Not hospitalized, no limitations on activities; |
| | 2. Not hospitalized, but new or increased |
| | limitation on activities and/or requiring new or |
| | increased home oxygen, CPAP, or BiPAP; |
| | 3. Hospitalized, not requiring supplemental |
| | oxygen - no longer requires ongoing medical |
| | care. |
| 2. To evaluate the proportion of subjects alive and | Proportion of subjects who satisfy one of the |
| without respiratory failure through Day 29. | following five categories from the ordinal scale |
| | through Day 29: |
| | 1. Not hospitalized, no limitations on activities; |
| | 2. Not hospitalized, but new or increased |
| | limitation on activities and/or requiring new or |
| | increased home oxygen, CPAP, or BiPAP; |
| | 3. Hospitalized, not requiring supplemental |
| | oxygen - no longer requires ongoing medical |
| | care; |
| | 4. Hospitalized, not requiring supplemental |
| | oxygen - requiring ongoing medical care |
| | (COVID-19 related or otherwise); |
| | 5. Hospitalized, requiring supplemental new or |
| | increased oxygen. |
| Other Secondary | |
| 1. To evaluate the clinical efficacy of different | Clinical outcome assessed using ordinal scale |
| investigational therapeutics as compared to the | daily while hospitalized and on Days 15, 22, |
| control arm as assessed by: | and 29. |
| Clinical Severity | |
| Ordinal scale: | |
| Time to an improvement of one | |
| category and two categories from Day 1 | |
| (baseline) using an ordinal scale. | |
| Mean change in the ordinal scale from | |
| Day 1 to Days 3, 5, 8, 11, 15, 22, and | |
| 29. | |
| Oxygenation: | Days of supplemental oxygen (if applicable) |
| Oxygenation use up to Day 29. | up to Day 29. |
| Incidence and duration of new oxygen | Supplemental oxygen concentration or flow |
| use during the study. | rate. |
| Oxygen requirement. | |
| Non-invasive ventilation/high flow oxygen: | Days of non-invasive ventilation/high flow |
| Non-invasive ventilation/high flow | oxygen (if applicable) up to Day 29. |
| oxygen use up to Day 29. | |
| Incidence and duration of new non- | |
| invasive ventilation or high flow oxygen | |
| use during the study. | |
| Invasive Mechanical Ventilation/ | Days of invasive mechanical |
| extracorporeal membrane oxygenation | ventilation/ECMO (if applicable) up to Day 29. |
| (ECMO): | |
| Ventilator/ECMO use up to Day 29. | |
| Incidence and duration of new | |
| mechanical ventilation or ECMO use | |
| during the study. | |
| Hospitalization: | Days of hospitalization and intensive care |
| Duration of hospitalization (days) | requirement up to Day 29. |
| Duration of ICU stay | |
| Mortality: | Date and cause of death (if applicable). |
| 14-day mortality. | |
| 29-day mortality. | |
| Time to death up to Day 29. | |
| Markers of inflammation and coagulation. | C-reactive protein (CRP), ferritin, d-dimer, |
| | fibrinogen, LDH, and troponin on Day 1, Days |
| | 3, 5, 8, and 11 (while hospitalized); and Days |
| | 15 and 29 (if attends in-person visit or still |
| | hospitalized). |
| 2. To evaluate the safety of different | SAEs. |
| investigational therapeutics as compared to the | Grade 3 and 4 AEs. |
| control arm as assessed by: | |
| Cumulative incidence of SAEs through Day | |
| 60. | |
| Cumulative incidence of Grade 3 and 4 | |
| clinical and/or laboratory AEs through Day | |
| 60. | |
| Discontinuation or temporary suspension of | Episodes of early discontinuation or |
| study product administration (for any | interruption of study product administration. |
| reason). | WBC with differential, hemoglobin, platelets, |
| Changes in white blood cell (WBC) count | creatinine, total bilirubin, ALT, AST, and INR |
| with differential, hemoglobin, platelets, | on Day 1; Days 3, 5, 8, and 11 (while |
| creatinine, total bilirubin, alanine | hospitalized); and Days 15 and 29 (if attends |
| aminotransferase (ALT), aspartate | in-person visit or still hospitalized). |
| aminotransferase (AST), and international | |
| normalized ratio (INR) over time (analysis of | |
| lab values in addition to AEs noted above). | |
| Exploratory | |
| 1. To evaluate the virologic efficacy of different | Qualitative and quantitative polymerase chain |
| investigational therapeutics as compared to the | reaction (PCR) for SARS-CoV-2 in OP swab |
| control arm as assessed by: | on Day 1; Days 3, 5, 8, and 11 (while |
| Percent of subjects with SARS-CoV-2 | hospitalized); and Days 15 and 29 (if attends |
| detectable in saliva (or best technology) | in-person visit or still hospitalized). |
| sample at Days 3, 5, 8, 11, 15, and 29. | Qualitative and quantitative PCR for SARS- |
| Quantitative SARS-CoV-2 virus in saliva (or | CoV-2 in blood on Day 1; Days 3, 5, 8, and 11 |
| best technology) sample at Days 3, 5, 8, 11, | (while hospitalized). |
| 15, and 29. | |
| Quantitative SARS-CoV-2 virus in blood at | |
| Days 3, 5, 8, and 11. | |
| 2. To evaluate the impact of study interventions | Proteomic analysis of cytokines and markers |
| on markers of inflammation and immune | of inflammation in serum and plasma |
| response. | including: IL1, IL-2R, IL-6, IL-8, IL-21, |
| | Pentraxin-3, citrullinated histone H3, MCP-1, |
| | TNF-b, Factor II |
| | Transcription, epigenetic, and molecular |
| | profiles of mRNA in peripheral blood |
| | mononuclear cells (PBMC). |
| | Phenotypic and responsiveness markers in |
| | PBMC. |
| 3. To evaluate post-baseline usage of key | Use of concomitant COVID-19 treatments up |
| concomitant COVID-19 treatments (e.g., steroids) | to Day 29. |
| in investigational therapeutic arms as compared | Incidence of progression to renal failure |
| to the control arm. | requiring dialysis at Day 29 |
| 4. To evaluate Patient Reported Outcomes. | SF-12 PCS and MCS scores at baseline, D 29 |
| | and D 60 |
| | EQ-5D-5L at baseline, D 29 and Day 60 |
| 5. To evaluate danicopan pharmacokinetics (PK) | Plasma and serum samples for PK and PD [APW |
| and pharmacodynamics (PD) in selected subjects | (Alternate pathway Wieslab), Factor B, Ba, Bb, |
| with COVID-19 at selected sites. | Factor D, C3, C4, C4d, C5, C5b9]: |
| | Day 1: pre-dose prior to 1^{st} and 2^{nd} dose; |
| | Day 3: pre-dose prior to 9th dose, 2 hr, 4 hr & |
| | 6 hr post-9th dose (Day 3 samples may be |
| | collected before and after a subsequent dose |
| | while hospitalized) |

Study Population

Approximately 200 male and non-pregnant female adults a 18 years of age or older with COVID-19 and who meet all eligibility criteria will be enrolled at up to 40 sites throughout the US. The target population should reflect the community at large. The estimated time from screening (Day −1 or Day 1) to end of study for an individual subject is approximately 60 days.

Subject inclusion and exclusion criteria must be confirmed by a clinician named on the delegation log. If there is any uncertainty, the site PI should make the decision on whether a potential subject is eligible for study enrollment. There is no exclusion for receipt of SARS-CoV-2 vaccine (experimental or licensed).

Inclusion Criteria

The inclusion criteria for the study are as follows:
1. Admitted to a hospital with symptoms suggestive of COVID-19 and requires ongoing medical care.
2. Subject (or legally authorized representative) provides informed consent prior to initiation of any study procedures.
3. Subject (or legally authorized representative) understands and agrees to comply with planned study procedures.
4. Male or non-pregnant female adult >/=18 years of age at time of enrollment.
5. Has laboratory-confirmed SARS-CoV-2 infection as determined by polymerase chain reaction (PCR) or other commercial or public health assay in any specimen, as documented by either of the following:
   PCR positive in sample collected <72 hours prior to screening; OR
   PCR positive in sample collected >/=72 hours but <14 days prior to screening AND non-improving or progressive disease suggestive of ongoing SARS-CoV-2 infection.
Note: if written documentation of the positive test result is not available at the time of enrollment (e.g., report came from other institution), the subject may be enrolled but the PCR should be repeated at the time of enrollment.
6. Illness of any duration, and requiring, just prior to randomization, supplemental oxygen (any flow), mechanical ventilation or Extracorporeal Membrane Oxygenation (ECMO) (ordinal scale category 5, 6, or 7).
7. Women of childbearing potential must agree to either abstinence or use at least one acceptable method of contraception* from the time of screening through 5 months post study intraperitoneal (IP) dosing. * Acceptable methods include barrier contraceptives (condoms or diaphragm) with spermicide, intrauterine devices (IUDs), hormonal contraceptives, oral contraceptive pills, and surgical sterilization.
8. Agrees not to participate in another blinded clinical trial (both pharmacologic and other types of interventions) for the treatment of COVID-19 through Day 29.

Exclusion Criteria

The exclusion criteria for the study are as follows:
1. Alanine aminotransferase (ALT) or aspartate aminotransferase (AST) >5 times the upper limit of normal.
2. Subjects with a low glomerular filtration rate (eGFR), specifically:
   a. Subjects with a glomerular filtration rate (eGFR) 20-30 mL/min are excluded unless in the opinion of the principal investigator (PI), the potential benefit of participation outweighs the potential risk of study participation.
   b. All subjects with a glomerular filtration rate (eGFR) <20 mL/min (including hemodialysis and hemofiltration) are excluded.
3. Pregnancy or breast feeding.
4. Anticipated discharge from the hospital or transfer to another hospital which is not a study site within 72 hours of enrollment.
5. Allergy to any study medication.
6. Received five or more doses of remdesivir prior to screening.
7. Received two or more doses of >60 mg of prednisone or equivalent in the 7 days prior to screening.
8. Received small molecule tyrosine kinase inhibitors, including Janus kinase (JAK) inhibitors (e.g., baricitinib, ibrutinib, acalabrutinib, imatinib, gefitinib), in the 4 weeks prior to screening.
9. Received monoclonal antibodies targeting cytokines (e.g., tumor necrosis factor (TNF) inhibitors, anti-IL-1 [e.g., anakinra, canakinumab], anti-IL-6 [e.g., tocilizumab, sarilumab, sitlukimab]), or T-cells (e.g., abatacept) in the 4 weeks prior to screening.
10. Received monoclonal antibodies targeting B-cells (e.g., rituximab, and including any targeting multiple cell lines including B-cells) in the 3 months prior to screening.
11. Received granulocyte-macrophage colony-stimulating factor (GM-CSF) agents (e.g., sargramostim) within 2 months prior to screening.
12. Received other immunosuppressants in the 4 weeks prior to screening and in the judgement of the investigator, the risk of immunosuppression with danicopan is larger than the risk of COVID-19.
13. Received any live vaccine in the 4 weeks prior to screening.
14. Known active tuberculosis.
15. Known history of HIV, Hepatitis B (HBV) or untreated hepatitis C (HCV) infection.
16. History of pulmonary alveolar proteinosis (PAP).
17. Has active malignancy, immunodeficiency, uncontrolled opportunistic infection, or uncontrolled cirrhosis.
18. Has a medical condition that could, in the judgment of the investigator, limit the interpretation and generalizability of trial results.
19. Positive test for influenza virus during the current illness (influenza testing is not required by protocol).
20. Previous participation in an ACTIV-5/BET trial.

Study Phase
Phase 2

Study Sites

There will be up to 40 sites throughout the US. Site selection will be determined by the epidemiology of COVID-19.

Study Interventions

All subjects will receive remdesivir as a 200-mg intravenous (IV) loading dose on Day 1, followed by a 100-mg once-daily IV maintenance dose during hospitalization up to a maximum of 10 total doses (i.e., loading+maintenance doses received during study and pre-study if applicable). The duration of dosing may be adjusted by the site similar to what is described in the emergency use authorization and based on a subject's clinical course and ultimate disease severity.

In addition to receiving remdesivir, subjects in the BET trial will be randomized to receive danicopan or placebo as follows:
Participants ≥70 years of age will receive 300 mg oral loading dose on Day 1, followed by 200 mg QID oral maintenance dose during hospitalization up to a maximum of 14 days. At the end of the treatment course (discharge or 14 days, whichever comes first), treatment will be tapered as followed: 200 mg TID for 2 days, followed by 200 mg BID for 2 days, until complete cessation (total treatment duration up to 18 days or 4 days after discharge).
Participants <70 years of age will receive 400 mg oral loading dose on Day 1, followed by 250 mg QID oral maintenance dose during hospitalization up to a maximum of 14 days. At the end of the treatment course (discharge or 14 days, whichever comes first), treatment will be tapered as followed: 250 mg TID for 2 days, followed by 250 mg BID for 2 days, until complete cessation (total treatment duration up to 18 days or 4 days after discharge).
It is anticipated that the majority of participants will not have received meningococcal vaccination within the 3 years prior to initiating treatment. If vaccination has not occurred, or cannot be confirmed, participants will receive appropriate antibiotics for prophylaxis against meningococcal infections. Appropriate antibiotics will be initiated prior to the first dose of the blinded study drug (danicopan or placebo) and will be continued for 2 days after its last dose.

Study Duration

This stage is anticipated to enroll over 3 months, with an additional 1 month of follow-up, and 2 months to lock the database.

Participant Duration

An individual subject will complete the study in about 60 days, from screening at Day −1 or 1 to follow-up on Day 60±3 days.

Schedule of Assessments

The Schedule of Assessments for this study is provided in Table 2 below.

TABLE 1
BET - Schedule of Assessments

| | | | Study | | | | |
|---|---|---|---|---|---|---|---|
| | | | Intervention Period | | | | |
| | Screen | Baseline | Daily until hospital | Follow-up Visits | | | |
| Day +/− Window | −1 or 1 | 1 | discharge (up to Day 29) | 15¹ ± 2 | 22² ± 3 | 29¹ ± 3 | 60² ± 3 |
| ELIGIBILITY | | | | | | | |
| Informed consent | X | | | | | | |
| Demographics & | X | | | | | | |
| Medical History | | | | | | | |
| Targeted physical exam | X | | | | | | |
| Review SARS-CoV-2 results | X | | | | | | |
| STUDY INTERVENTION | | | | | | | |
| Randomization | | X | | | | | |
| Administration of | | Danicopan or placebo: | | | | | |
| investigational agent | | Participants ≥70 years of age: | | | | | |
| | | 300 mg PO loading dose; 200 | | | | | |
| | | mg QID maintenance dose for | | | | | |
| | | the duration of hospitalization | | | | | |
| | | (up to 14 days). At the end of | | | | | |
| | | treatment (discharge or 14 | | | | | |
| | | days), study drug is tapered: | | | | | |
| | | 200 mg TID for 2 days, followed | | | | | |
| | | by 200 mg BID for 2 days. | | | | | |
| | | Participants <70 years of age: | | | | | |
| | | 400 mg PO loading dose; 250 | | | | | |
| | | mg QID maintenance dose for | | | | | |
| | | the duration of hospitalization | | | | | |
| | | (up to 14 days). At the end of | | | | | |
| | | treatment (discharge or 14 | | | | | |
| | | days), study drug is tapered: | | | | | |
| | | 250 mg TID for 2 days, followed | | | | | |
| | | by 250 mg BID for 2 days. | | | | | |
| | | Remdesivir: IV daily for 5-10 | | | | | |
| | | days or until discharge. | | | | | |
| Administration of | | Prophylactic antibiotics if not | | | | | |
| prophylactic antibiotics | | vaccinated against meningococcal | | | | | |
| | | infection within the last 3 years | | | | | |
| | | Initiate prior to the first dose of | | | | | |
| | | investigational agent; continue | | | | | |
| | | for 2 days after the last dose of | | | | | |
| | | investigational agent | | | | | |
| STUDY PROCEDURES | | | | | | | |
| Vital sign and NEWS | | X⁵ | | | | | |
| score^{3,4} | | | | | | | |
| Clinical data collection⁴ | | X⁵ | Daily until discharge | X | X | X | X⁶ |
| Adverse event evaluation | | X⁵ | Daily until discharge | X | X | X | X |
| Concomitant medication | | X⁵ | Day −7 until discharge | X | X | X | |
| review⁷ | | | | | | | |
| SF-12 & EQ-5D-5L | | X | | | | X | X |
| SAFETY LABORATORY | | | | | | | |
| Safety hematology, | X⁹ | X⁵ | Day 3, 5, 8, 11 (all ± | X | | X | |
| chemistry, and liver | | | 1 day) if hospitalized¹⁰ | | | | |
| tests⁸ | | | | | | | |
| Pregnancy test for females | X⁹ | | | | | | |
| of childbearing potential | | | | | | | |
| RESEARCH LABORATORY | | | | | | | |
| Oropharyngeal swab¹¹ | | X⁵ | Day 3, 5, 8, 11 (all ± | X | | X | |
| | | | 1 day) if hospitalized | | | | |
| Blood draw for serum | | X⁵ | Day 3, 5, 8, 11 (all ± | X | | X | |
| and plasma. | | | 1 day) if hospitalized | | | | |
| Specific testing is | | | | | | | |
| as follows: | | | | | | | |
| PCR SARS-CoV-2 | | X⁵ | Day 3, 5, 8, 11 (all ± | | | | |
| | | | 1 day) if hospitalized | | | | |
| proteomic analysis | | X⁵ | Day 3, 5, 8, 11 (all ± | X | | X | |
| (including specifically | | | 1 day) if hospitalized | | | | |
| for IL1, IL-2R, IL-6, IL- | | | | | | | |
| 8, IL-21, Pentraxin-3, | | | | | | | |
| citrullinated histone | | | | | | | |
| H3, MCP-1, TNF-b, | | | | | | | |
| Factor II | | | | | | | |
| Danicopan pharmacokinetics | | Day 1: pre-dose prior to 1st and | | X | | X | |
| and pharmacodynmics¹⁴ | | 2nd dose; | | | | | |
| | | Day 3: pre-dose prior to 9th dose, | | | | | |
| | | 2 hr, 4 hr & 6 hr post-9th dose (Day | | | | | |
| | | 3 samples may be collected before | | | | | |
| | | and after a subsequent dose while | | | | | |
| | | hospitalized) | | | | | |
| Plasma and Serum for | | X⁵ | Day 3, 5, 8, 11 (all ± | X | | X | |
| secondary research¹² | | | 1 day) if hospitalized | | | | |
| Blood for RNA | | X⁵ | Day 3, 8 (all ± 1 | X | | X | |
| | | | day) if hospitalized | | | | |
| Blood for PBMC¹³ | | X⁵ | Day 3, 8 (all ± 1 | X | | X | |
| | | | day) if hospitalized | | | | |

Notes:
¹In-person visits are preferred but recognizing quarantine and other factors may limit the subject's ability to return to the site for the visit. In this case, the visit may be performed by phone.
If still hospitalized at Day 15 and 29 or returns to the site for an in-person visit: assess adverse events, collect clinical data, vital signs, safety laboratory tests, and research laboratory samples (OP swab and blood) as able.
If phone call only on Days 15 and 29 and all Day 22 and Day 60 visits: assess adverse events, clinical status (ordinal scale), readmission to a hospital, and mortality only.
²Day 22 and Day 60 visits performed by phone.
³Vital signs include temperature, systolic blood pressure, heart rate, respiratory rate, O2 saturation and level of consciousness. In addition, height and weight are obtained only at baseline (height can be self-reported). Vital signs collected as part of standard care may be used.
⁴Refer to the section titled “Study Assessments and Procedures” for details of clinical data to be collected including ordinal score, NEWS, oxygen requirement, mechanical ventilator requirement, etc.
⁵Baseline assessments should be performed prior to first infusion. Laboratory tests performed as part of routine clinical care in the 24 hours prior to first dose will be accepted for the baseline safety laboratory tests. Baseline may be the same as the screening laboratory tests if obtained in the 24 hours prior to first dose.
⁶Ordinal score only.
⁷Steroids and other concomitant therapies intended as specific treatment of COVID-19, as well as all biologics, will be assessed from 7 days prior to enrollment to Day 29. All other concomitant medications will be assessed from 7 days prior to enrollment to Day 15 or upon discharge, whichever comes first. Antibiotics that will provide prophylaxis against meningococcal infections will be assessed until the end of the prescribed course.
⁸Safety laboratory tests include WBC with differential, hemoglobin, platelets, creatinine, total bilirubin, ALT, AST, alkaline phosphatase, and INR.
⁹Screening laboratory tests include: ALT, AST, creatinine (and calculate an estimated glomerular filtration rate (eGFR) the formula used is determined by the sites, but should be consistent throughout the study), and urine or serum pregnancy test for females of child-bearing potential. Laboratory tests performed as part of routine clinical care in the 48 hours prior to enrollment will be accepted for determination of eligibility.
¹⁰Any laboratory tests performed as part of routine clinical care within the specified visit window can be used for safety laboratory testing.
¹¹Oropharyngeal swabs are preferred, but if these are not obtainable, saliva or nasopharyngeal or nasal swabs may be substituted.
¹²To include markers of inflammation and coagulation: CRP, ferritin, fibrinogen, d-dimer, troponin.
¹³Only collected at selected sites capable of collecting PBMC.
¹⁴The first approximately 20 subjects included in this study will be involved in a PK/PD analysis to confirm the dosing regimen.

Justification for Dose

Justification for Dose of Remdesivir

The dose of remdesivir used in this study will be the same dose shown to be efficacious in the ACTT-1 clinical trial (Beigel et al. Remdesivir for the treatment of Covid-19—final report. N Engl J Med. 2020; 383(19): 1813-1826), and is the US FDA approved doses. The duration of dosing may be adjusted by the site according to clinical severity. The maximum number of doses to be given during hospitalization is ten doses. This includes the loading dose and all maintenance doses given during the study and pre-study if applicable.

Justification for Dose of Danicopan

The current population PK model, developed using all the current available clinical PK data, indicates that body weight and age are 2 important factors impacting PK. Greater body weight is associated with lower drug concentrations, whereas greater age is associated with higher drug concentrations, particularly for individuals ≥50 years of age. Therefore, simulations have been conducted in 3 age groups, <50, 50 to 69, and a 70 years of age with their respective body weight distributions according to the Centers for Disease Control and Prevention database.

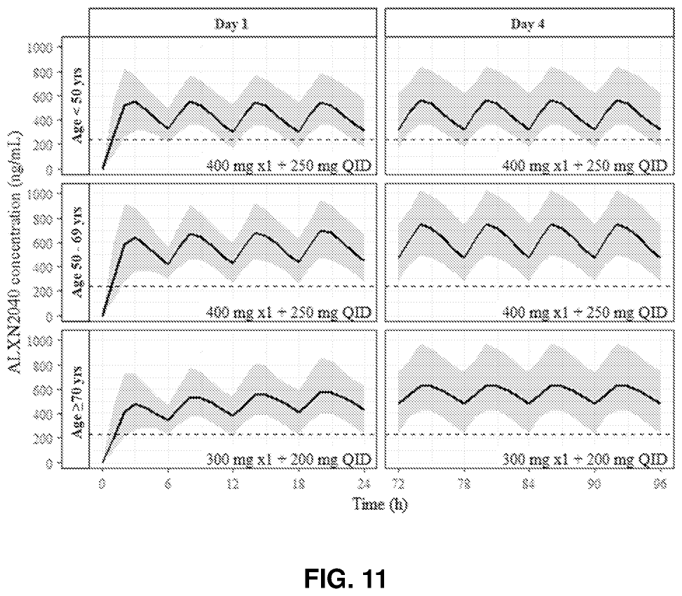

In this study, the 300 mg loading dose followed by 200 mg QID dose is proposed for age ≥70 years old, and the 400 mg loading dose followed by 250 mg QID dose is proposed for age <70 years old, based on the simulation results. Plasma PK exposures for these proposed doses are predicted to achieve a 90% APH inhibition in the majority of participants during the entire dose interval (FIG. 11). Considering the overall safety profile of danicopan and the transient nature of liver enzymes elevation observed in the clinical program thus far, coupled with the laboratory monitoring schedule in this study, these doses provide the optimal benefit to risk balance.

The predicted systemic PK exposures for the proposed dose regimens in individuals in 3 age groups at Day 1 and at Day 4 (steady state) are shown in Table 3.

TABLE 3
Simulated PK Exposure Summary Parameters for Danicopan

| | | | | | | | |
|---|---|---|---|---|---|---|---|
| | Age | | | | | | Geometric |
| Dose | (yr) | Parameter | n | Mean (SD) | Median (range) | CV | mean |

Day 1

| | | | | | | | | | |
|---|---|---|---|---|---|---|---|---|---|
| 300 mg × 1 + | | AUC (h*ng/mL) | 136 | 11005 | (2359) | 10725 | (5571-18081) | 21 | 10757 |
| 200 mg QID | ≥70 | Cmax (ng/mL) | 136 | 613 | (141) | 588 | (292-1020) | 23 | 597 |
| | | Ctrough (ng/mL) | 136 | 348 | (74) | 346 | (193-546) | 21 | 341 |
| 400 mg × 1 + | | AUC (h*ng/mL) | 234 | 10445 | (2380) | 10223 | (4837-19498) | 23 | 10180 |
| 250 mg QID | <50 | Cmax (ng/mL) | 234 | 609 | (152) | 601 | (313-1163) | 25 | 590 |
| | | Ctrough (ng/mL) | 234 | 337 | (90) | 327 | (149-677) | 27 | 326 |
| | | AUC (h*ng/mL) | 126 | 12929 | (2458) | 12848 | (8225-20978) | 19 | 12699 |
| | 50-69 | Cmax (ng/mL) | 126 | 723 | (155) | 726 | (427-1163) | 21 | 707 |
| | | Ctrough (ng/mL) | 126 | 423 | (87) | 419 | (234-735) | 21 | 414 |

Day 4

| | | | | | | | | | |
|---|---|---|---|---|---|---|---|---|---|
| 300 mg × 1 + | | AUC (h*ng/mL) | 136 | 13961 | (3797) | 13616 | (6582-28083) | 27 | 13472 |
| 200 mg QID | ≥70 | Cmax (ng/mL) | 136 | 666 | (171) | 640 | (301-1283) | 26 | 644 |
| | | Ctrough (ng/mL) | 136 | 474 | (156) | 479 | (192-1013) | 33 | 449 |
| 400 mg × 1 + | | AUC (h*ng/mL) | 234 | 11464 | (3110) | 10787 | (4724-23420) | 27 | 11067 |
| 250 mg QID | <50 | Cmax (ng/mL) | 234 | 583 | (144) | 564 | (264-1102) | 25 | 566 |
| | | Ctrough (ng/mL) | 234 | 348 | (130) | 317 | (118-794) | 37 | 325 |
| | | AUC (h*ng/mL) | 126 | 15139 | (3407) | 15039 | (9234-27171) | 23 | 14771 |
| | 50-69 | Cmax (ng/mL) | 126 | 741 | (156) | 749 | (441-1234) | 21 | 724 |
| | | Ctrough (ng/mL) | 126 | 492 | (148) | 470 | (217-986) | 30 | 471 |

Abbreviations: AUC = area under the curve; Cmax = maximal serum drug concentration; Ctrough = minimal serum drug concentration; CV = coefficient of variation; QID = quarter in die (four times daily).

The predicted systemic PK exposures for these doses/dosing regimens are expected to be reliable as the population PK model uses extensive PK data from multiple clinical studies in participants with a wide age range (16.9 to 72 years) and body weight range (43.4 to 130 kg), under the assumption that the danicopan PK and PK-PD relationship are the same between healthy volunteers and participants with COVID-19.

Study Product(s) and Administration—Remdesivir Danicopan, and Placebo

Study Product Description

Remdesivir Component:

Remdesivir is a single diastereomer monophosphoramidate prodrug designed for the intracellular delivery of a modified adenine nucleoside analog GS-441524. In addition to the active ingredient, the solution formulation of remdesivir contains the following inactive ingredients: water for injection, SBECD, and hydrochloric acid and/or sodium hydroxide.

Danicopan Component:

The danicopan drug product is a film-coated, immediate release tablet in strengths of 50 mg and 100 mg, intended for oral administration. The tablet contains the drug substance, lactose, microcrystalline cellulose, croscarmellose sodium, sodium lauryl sulphate, magnesium stearate, colloidal silicon dioxide and hypromellose acetate succinate. The tablet coating components are polyvinyl alcohol, titanium dioxide, macrogol/polyethylene glycol and talc.

Danicopan Matching Placebo:

The supplied matching placebo of danicopan is identical in physical appearance to the active oral formulation and contains the same inactive ingredients.

Dosing and Administration

All subjects will receive remdesivir as a 200 mg intravenous (IV) loading dose on Day 1, followed by a 100 mg once-daily IV maintenance dose for the duration of the hospitalization up to a 10-day total course. If subjects already received the loading dose prior to study enrollment, then start at 100 mg/day on Day 1. Any doses of remdesivir given prior to enrollment will be counted, so the total duration of remdesivir (i.e. pre-enrollment+on this trial) is 10 days (i.e., a maximum of 10 total infusions). Any doses of remdesivir were administered prior to study enrollment should be documented in on the eCRF as a concomitant medication given prior to Day 1. The duration of dosing may be adjusted by the site similar to what is described in the package insert and based on a subject's clinical course and ultimate disease severity.

Any dose of remdesivir that is delayed may be given later that calendar day. Any dose or remdesivir that is missed (not given that calendar day) is not made up. The treatment course continues as described above even if the subject becomes PCR negative.

In addition to receiving remdesivir, subjects in the BET trial will be randomized to receive danicopan or placebo as follows:
Patients ≥70 years of age: subjects will receive 300 mg oral (PO) loading dose followed by 200 mg QID dose as maintenance dose for the duration of the hospitalization up to a 14-day total course. At the end of the treatment course (discharge or 14 days, whichever comes first), treatment will be tapered as follows: 200 mg TID for 2 days, followed by 200 mg BID for 2 days, until complete cessation (total treatment duration up to 18 days or 4 days after discharge).
Patients <70 years of age: subjects will receive 400 mg PO loading dose followed by 250 mg QID dose as maintenance dose for the duration of the hospitalization up to a 14-day total course. At the end of the treatment course (discharge or 14 days, whichever comes first), treatment will be tapered as follows: 250 mg TID for 2 days, followed by 250 mg BID for 2 days, until complete cessation (total treatment duration up to 18 days or 4 days after discharge).

It is anticipated that the majority of participants will not have received meningococcal vaccination within the 3 years prior to initiating treatment. If vaccination has not occurred, or cannot be confirmed, patients will receive appropriate antibiotics for prophylaxis against meningococcal infections. Appropriate antibiotics will be initiated prior to the first dose of the blinded study drug (danicopan or placebo) and will be continued for 2 days after its last dose. At least 3 hours between administration of remdesivir and danicopan or placebo are preferred if possible (to prevent Cₘₐₓ from occurring simultaneously). However, timely administration of both products is most important, and if the interval between products is likely to cause delays for either product, it can be omitted.

If the dose of danicopan is delayed, it should be given as soon as practical, unless the next dose is due within 3 hours, in which case the dose should be skipped.

Danicopan and its placebo may be administered via nasogastric tube to patients who are unable to swallow the study drug orally. Further details will be provided in the pharmacy manual.

Dose Escalation

Dose escalation is possible in this study.

Dose Modifications

Remdesivir Component:

The infusion should be held and not given if the subject is found to have any of the following laboratory values:
eGFR decreases to <20 mL/min
   Remdesivir infusion will resume when the eGFR increases to a 20 mL/min and the potential benefit of giving remdesivir outweighs the potential risk.
   If renal function worsens during the study to the point that they require hemodialysis or hemofiltration, remdesivir will be discontinued.
ALT and/or AST increases to >5 times ULN
   Remdesivir infusion will be held and not be restarted until the ALT and AST ≤5 times ULN.

Concomitant Therapy

It is anticipated that the majority of participants will not have received meningococcal vaccination within the 3 years prior to initiating treatment. If vaccination has not occurred, or cannot be confirmed, patients will receive appropriate antibiotics for prophylaxis against meningococcal infections. Appropriate antibiotics will be initiated prior to the first dose of the blinded study drug (danicopan or placebo) and will be continued for 2 days after the last dose of the investigational therapy. Appropriate prophylaxis may be provided through antibiotics that the study participant might be receiving for another indication such as bacterial pneumonia.

Prohibited Concomitant Therapy

Receipt of any exclusionary treatments or medications prior to screening will be assessed at screening to determine eligibility as described in the exclusion criteria.

The following medications are prohibited during this study:
Small molecule tyrosine kinase inhibitors (e.g., baricitinib, ibrutinib, acalabrutinib, imatinib, gefitinib).
Monoclonal antibodies targeting cytokines (e.g., TNF inhibitors, anti-IL-1 [e.g., anakinra or canakinumab], anti-IL-6 [e.g., tocilizumab, sarilumab, or sitlkimab]), or T-cells (e.g., abatacept).
Monoclonal antibodies targeting B-cells (e.g., rituximab, and including any targeting multiple cell lines including B-cells).
GM-CSF agents (e.g., sargramostim).
Other immunosuppressants which, in the judgement of the investigator, pose risk of immunosuppression in combination with danicopan that is larger than the risk of COVID-19.
Chloroquine or hydroxychloroquine use for the treatment of COVID-19.

A recent study found that chloroquine antagonizes remdesivir in a dose dependent manner as evidenced by an increase in the median effective dose (EC50) for remdesivir with increasing chloroquine concentration. Another in vitro study found that chloroquine induces a dose dependent inhibition of the formation of the active nucleoside triphosphate metabolite of remdesivir. Thus, chloroquine or hydroxychloroquine use for the treatment of COVID-19 is prohibited during the study.

Concomitant use of any other experimental treatment or off-label use of marketed medications intended as specific treatment for COVID-19 or SARS-CoV-2 infection, and not specified in local written guidelines or the NIH COVID-19 Treatment Guidelines are prohibited.

Study Assessments and Procedures

Screening Procedures

Screening procedures may be done from Day −1 to Day 1. However, in many cases all the screening procedures can be done in less than 24 hours. If that is the case, Day 1 pre-study product administration baseline assessments, specimen collection and the initial study product administration can occur on the same calendar day as the screening procedures.

After the informed consent, the following assessments are performed to determine eligibility and obtain baseline data:
Confirm the positive SARS-CoV-2 test result (per inclusion criteria).
Take a focused medical history, including the following information. Additional information may be needed based on risk profile of the study product and the exclusion criteria (e.g., recent live vaccine history). The minimum history includes:
   Day of onset of COVID-19 signs and symptoms.
   History of chronic medical conditions including chronic oxygen requirement prior to onset of COVID-19.
   History of medication allergies.
   Medications and therapies for this current illness taken in the 7 days prior to enrollment.
   Ask if they are participating in another clinical trial or plan to enroll in another clinical trial in the next 30 days.
Women of childbearing potential should be counseled to either practice abstinence or use at least one primary form of contraception not including hormonal contraception from the time of screening through Day 29. Women should be confirmed to not be breastfeeding.
   Note: If a woman is either postmenopausal (i.e., has had 212 months of spontaneous amenorrhea) or surgically sterile (i.e., has had a hysterectomy, bilateral ovariectomy (oophorectomy), or bilateral tubal ligation), she is not considered to be of childbearing potential.
Height and weight (height can be self-reported).
Results of most recent radiographic imaging (chest x-ray or CT scan).
SpO2 on room air (not needed if on supplemental oxygen, mechanical ventilation, or ECMO).
Blood for screening laboratory evaluations if not done as part of routine clinical care in the 48 hours prior to enrollment. Additional screening laboratory evaluations may be added based on the risk profile of the study product for a given stage of the study. Please see appendices for stage-specific details. The minimum screening laboratory evaluations include:
   ALT.
   AST.
   Creatinine (and calculate eGFR).
      Any automated calculation by the clinical laboratory or published formula for this calculation is acceptable. The site should select a formula to be used for all subjects enrolled at the site for the duration of the study.
   Urine or serum pregnancy test (in women of childbearing potential).

Clinical screening laboratory evaluations will be performed locally by the site laboratory.

The overall eligibility of the subject to participate in the study will be assessed once all screening values are available. Complete the inclusion and exclusion criteria checklists on the day of enrollment as these forms include data needed to register all potential subjects in the electronic data capture (EDC) system. The screening process can be suspended prior to complete assessment at any time if exclusions are identified by the study team.

Study subjects who qualify will be randomized in the interactive response technology system (IRT) system, and all others will be registered as screen failures only in the EDC system. The study team has 24 hours to complete Day 1 baseline assessments prior to the first study product administration including the collection of OP swab and blood, assessment of the ordinal scale.

Efficacy Assessments

For all baseline assessments and follow-up visits, refer to the Schedule of Assessments (SOA) for procedures to be completed, and details below for each assessment.

Laboratory tests performed as part of routine clinical care in the 24 hours prior to first dose will be accepted for the baseline laboratory tests. Baseline may be the same as the screening laboratory tests if obtained in the 24 hours prior to first dose.

Measures of Clinical Support, Limitations, and Infection Control

The subject's clinical status will be captured on each study day while hospitalized up until and including Day 29. If a subject is discharged prior to Day 15, clinical status is captured on Days 15 and 29 as an outpatient if the subject returns for an in-person clinic visit or by phone if an in-person visit is not possible. Clinical status will also be captured on the Day 22 phone visit. Clinical status is largely measured by the ordinal scale and the NEWS. Unlike the NEWS, the ordinal scale can also be evaluated over the phone if the discharged subject is unable to return for in-person visits on Day 15 and 29 as well as on Day 22.

Ideally, the ordinal scale is completed concurrently with the NEW Score just prior to study product administration. The following measures are recorded for the ordinal scale:
Hospitalization.
Oxygen requirement.
Non-invasive mechanical ventilation (via mask) requirement.
Invasive mechanical ventilation (via endotracheal tube or tracheostomy tube) requirement.
ECMO requirement.
Ongoing medical care preventing hospital discharge (COVID-19 related or other medical conditions).
Limitations of physical activity (self-assessed).
Isolated for infection control purposes.

Ordinal Scale

The ordinal scale is the primary measure of clinical outcome. The scale used in this study is as follows:
1. Not hospitalized, no limitations on activities;
2. Not hospitalized, limitations on activities and/or requiring home oxygen*;
3. Hospitalized, not requiring supplemental oxygen*—no longer requires ongoing medical care;
4. Hospitalized, not requiring supplemental oxygen*—requiring ongoing medical care (COVID-19 related or otherwise);
5. Hospitalized, requiring supplemental oxygen*;
6. Hospitalized, on non-invasive ventilation or high flow oxygen devices;
7. Hospitalized, on invasive mechanical ventilation or ECMO;
8. Death. *For subjects on chronic supplemental oxygen, this pertains to an increased oxygen requirement above baseline.

National Early Warning Score (NEWS)

NEWS has demonstrated an ability to discriminate subjects at risk of poor outcomes (Smith et al. A Comparison of the Ability of the Physiologic Components of Medical Emergency Team Criteria and the U.K. National Early Warning Score to Discriminate Patients at Risk of a Range of Adverse Clinical Outcomes. Crit Care Med. 2016; 44(12): 2171-2181). This score is based on 7 clinical parameters (see Table 4). The NEWS is being used as an efficacy measure. The NEW Score should be evaluated daily while hospitalized and on Days 15 and 29. It can be performed concurrently with the Ordinal Scale. This should be evaluated at a consistent time for each study day and prior to administration of study product. The 7 parameters can be obtained from the hospital chart or electronic medical record (EMR) using the last measurement prior to the time of assessment and a numeric score is given for each parameter (e.g., a RR of 9 is one point, oxygen saturation of 92 is two points). This is recorded for the day obtained (i.e., on Day 3, the vital signs and other parameters from Day 3 are used to obtain NEW Score for Day 3). ECMO and mechanically ventilated subjects should be assigned a score of 3 for RR (RR<8) regardless of the ventilator setting. Subjects on ECMO should get a score of 3 for heart rate since they are on cardiopulmonary bypass.

TABLE 4
National Early Warning Score (NEWS)

| PHYSIOLOGICAL | | | | | | | |
|---|---|---|---|---|---|---|---|
| PARAMETERS | 3 | 2 | 1 | 0 | 1 | 2 | 3 |
| Respiration Rate | ≤8 | | 9-11 | 12-20 | | 21-24 | ≥25 |
| Oxygen Saturations | ≤91 | 92-93 | 94-95 | ≥96 | | | |
| Any Supplemental | | Yes | | No | | | |
| Oxygen | | | | | | | |
| Temperature | ≤35.0 | | 35.1-36.0 | 36.1-38.0 | 38.1-39.0 | ≥39.1 | |
| Systolic BP | ≤90 | 91-100 | 101-110 | 111-219 | | | ≥220 |
| Heart Rate | ≤40 | | 41-50 | 51-90 | 91-110 | 111-130 | ≥131 |
| Level of | | | | A | | | V, P, or U |
| Consciousness | | | | | | | |

Level of consciousness = alert (A), and non-alert and arousable only to voice (V) or pain (P), and unresponsive (U).

Exploratory Assessments

Viral Load and/or Shedding

As outlined in the SOA, OP swabs and plasma and serum will be collected on Day 1; and Days 3, 5, 8, and 11 (while hospitalized); and OP swabs and plasma and serum on Day 15 and 29 (if attends an in-person visit or still hospitalized). These assays are not developed yet, and the ability to test samples at one central lab is not clear. Therefore, while viral load/shedding is thought to be an important endpoint, considering the limitations above, it is listed as an exploratory endpoint.

OP swabs are preferred, but if these are not obtainable, nasopharyngeal (NP) or nasal swabs may be substituted. Due to limited lack of swabs and other supplies at some sites and limitations on personal protective equipment (PPE), the inability to obtain these samples are not considered protocol deviations and should be documented in the subject's record.

If virology assays can be set up with enough numbers of specimens tested, these data will be submitted as part of the Clinical Study Report (CSR). This may be submitted separately, as a supplemental CSR.

Samples collected for viral assessment may be probed for the emergence of antiviral resistance at a future date. These data, if available, may be submitted as a supplement report.

The schedule of assessments (Table 2) lists several research laboratory samples to be collected. It is preferred that these samples are collected and sent to the NIAID repository to be tested in one central laboratory. Current US Centers for Disease Control and Prevention (CDC) guidance is these samples can be processed in a Biosafety Laboratory (BSL) 2 environment. However, institutions may impose restrictions on processing the samples (i.e., they may require BSL-3) or there may be restrictions on sending samples. In these circumstances, the following apply:

Blood for PCR SARS-CoV-2:
If the samples can be processed but cannot be sent to the repository, the samples may be stored locally.
The sponsor may elect to have some or all of these samples run locally, pending confirmation of the assays to be used and the qualifications of the local laboratory. The sponsor will work with the site to determine when this could occur and hos these data can be imported into the study database.
If a BSL-3 environment is needed for processing these samples, these samples may be omitted.

Oropharyngeal, Nasopharyngeal, or Nasal Swab:
If the samples can be processed by cannot be sent to the repository, the samples may be stored locally.
The sponsor may elect to have some or al of these samples run locally, pending confirmation of the assays to be used and the qualifications of the local laboratory. The sponsor will work with the site to determine when this could occur and how these data can be importuned into the study database.
If a BSL-3 environment is needed for processing these samples, these samples may be omitted and documented each time omitted.

Blood for Serum (for Secondary Research):
If the samples can be processed and but not sent to the repository, the samples may be stored locally.
If a BSL-3 environment is needed for processing these samples, these samples may be omitted and documented each time omitted.

Immunology Studies:

As outlined in the SOA, plasma and blood will be collected on Days 1, 3, and 8 (while hospitalized) and on Days 15 and 29 (if attends an in-person visit or still hospitalized) for evaluation of cytokines, proteomic analysis of inflammation and cytokines, transcriptome analysis and PBMC assessment. If a BSL-3 environment is needed for processing of these samples, these samples may be omitted and documented each time omitted.

Transcriptome analysis and PBMC assessment involve genetic sequencing, however this study will not involve genetic tests intended to discover disease-determining genes. Study analyses could potentially result in medically relevant incidental findings. Additionally, in the future, novel disease-associated phenotypes may be discovered that might be identified in samples stored under this study. Many research laboratory tests are not certified by the Clinical Laboratory Improvement Amendments (CLIA), so generated genetic data cannot be meaningfully interpreted outside the narrow focus of the study and will not be routinely returned to subjects or their physicians. If a clinically significant finding is discovered and a CLIA-certified test is available for confirmation, the PI (or designee) will contact the subject to inform them of the finding and counsel them on confirming the result through a clinical provider.

Additional exploratory assessments include:
Qualitative and quantitative polymerase chain reaction (PCR) for SARS-CoV-2 in OP swab on Day 1; Days 3, 5, 8, and 11 (while hospitalized); and Days 15 and 29 (if attends in-person visit or still hospitalized).
Qualitative and quantitative PCR for SARS-CoV-2 in blood on Day 1; Days 3, 5, 8, and 11 (while hospitalized).
Plasma cytokines or other inflammatory markers on Days 1, 3, 8, 15, and 29 (if attends in-person visit or still hospitalized).
Plasma proteomic analysis of cytokines and markers of inflammation in serum and plasma on Days 1, 3, 8, 15, and 29 (if attends in-person visit or still hospitalized). These include: IL1, IL-2R, IL-6, IL-8, IL-21, Pentraxin-3, citrullinated histone H3, MCP-1, TNF-b, and Factor II.
Blood RNA transcriptome analysis on Days 1, 3, 8, 15, and 29 (if attends in-person visit or still hospitalized).
PBMC assessment for phenotype and functional reactivity on Days 1, 3, 8, 15, and 29 (if attends in-person visit or still hospitalized; collected only at sites capable of collecting and processing PBMC).
Plasma samples for PK:
   Day 1: pre-dose prior to 2^{nd} dose;
   Day 3: pre-dose prior to 9^{th} dose, 2 hr, 4 hr, and 6 hr post-9^{th} dose (Day 3 samples may be collected before and after a subsequent dose while hospitalized)
Patient-reported outcomes:
   Short-Form (SF)-12: The SF-12 is a validated health-related quality of life (HR QoL) instrument that is widely used across a broad spectrum of disease indications. Adapted from the 36-item SF survey that was designed to evaluate physical and mental health status, the SF-12 survey contains only 12 questions but covers the same 8 domains. There is a further stratification into 2 summary measures (Physical Component Summary [PCS-12] and Mental Component Summary [MCS-12]) as specified:
      PCS-12:
         General health (1 item)
         Physical functioning (2 items)
         Role physical (2 items)
         Body pain (1 item)
      MCS-12
         Vitality (1 item)
         Social functioning (1 item)
         Role emotional (2 items)
         Mental health (2 items)
   The PCS-12 and MCS-12 summary measures are scored using a norm-based method (i.e., mean=5, SD=10) (Jenkinson et al., J Public Health Med. 1997; 19(2): 179-186). A PCS-12 or MCS-12 score of 50 indicates an average score with respect to a healthy population. Scores lower than 50 reflect less than average health and scores greater than 50 reflect better than average health (Ware, et al., The Health Institute, New England Medical Center. 1995). The SF-12 assumes a recall of 1 week before responding to questions. The survey is anticipated to be completed in several minutes and can be completed by the participant or via an interviewer (in-person or over the phone).
   EuroQol 5 dimension-6 severity level (EQ-6D-6L): The EQ-5D-5L questionnaire is a brief, validated, HR-QoL instrument that is intended to assess the participant's health status at the time of administration. The questionnaire contains 5 dimensions (mobility, self-care, usual activities, pain/discomfort, and anxiety/depression), each of which includes 5 response variables (no problems, slight problems, moderate problems, severe problems, and extreme problems) (EuroQol Research Foundation, 2019). There is no summary score generated upon completion, but rather a 5-digit profile (termed “health state”) based on each of the dimensions that can be further converted to a single numerical score (index value). Value sets (a collection of index values) have been derived for multiple countries/regions. A vertical visual analogue scale (VAS) is included for participants to indicate a self-rated estimate of their health. The VAS ranges from 100 (best health you can imagine) to 0 (worst health you can imagine). The EQ-5D-5L questionnaire and VAS are anticipated to be completed in several minutes and can be completed by the participant, via an interviewer (in-person or over the telephone); or via proxy.

### Numbered Embodiments

Embodiment 1. A method of treating a betacoronavirus infection in a human subject, the method comprising administering to the human subject a therapeutically effective amount of at least one complement factor D (CFD) inhibitor.
Embodiment 2. The method of embodiment 1, wherein the at least one CFD inhibitor is selected from Compound 1, Compound 2, Compound 3, and Compound 4:

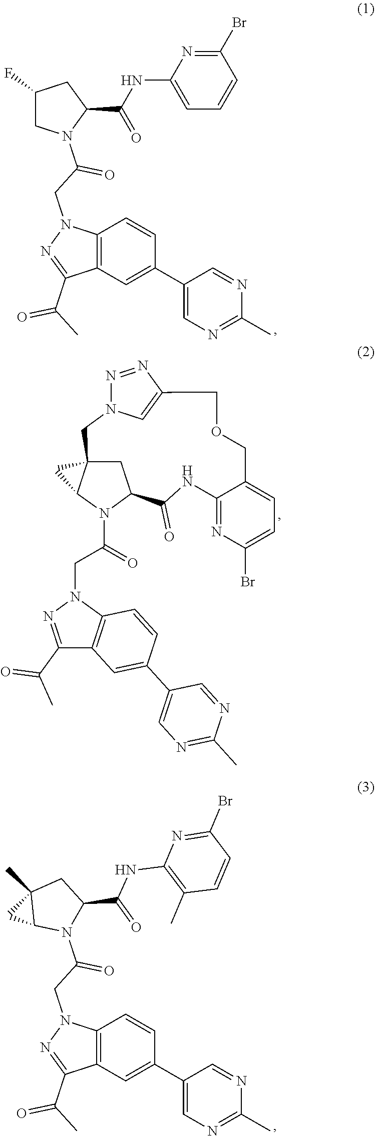

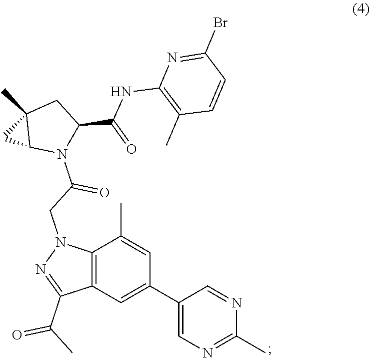

and pharmaceutically acceptable salts thereof.
Embodiment 3. The method of embodiment 2, wherein the at least one CFD inhibitor is Compound 1:

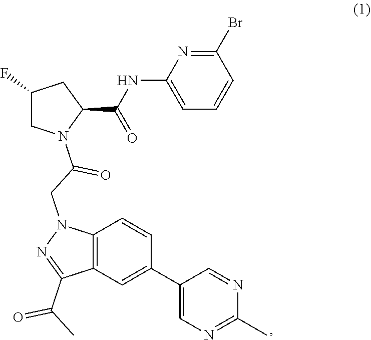 [SMILES: CC(=O)c1nn(CC(=O)N2C[C@H](F)C[C@H]2C(=O)Nc2cccc(Br)n2)c2ccc(-c3cnc(C)nc3)cc12]

or a pharmaceutically acceptable salt thereof.
Embodiment 4. The method of embodiment 2, wherein the at least one CFD inhibitor is Compound 2:

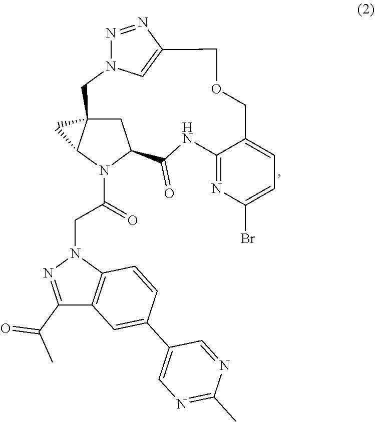 [SMILES: CC(=O)c1nn(CC(=O)N2[C@H]3C[C@]4(C[C@@H]24)Cn2cc(nn2)COCc2ccc(Br)nc2NC3=O)c2ccc(-c3cnc(C)nc3)cc12]

or a pharmaceutically acceptable salt thereof.
Embodiment 5. The method of embodiment 2, wherein the at least one CFD inhibitor is Compound 3:

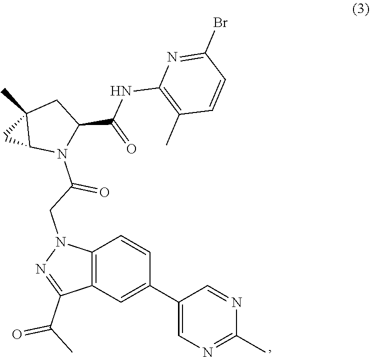

or a pharmaceutically acceptable salt thereof.
Embodiment 6. The method of embodiment 2, wherein the at least one CFD inhibitor is Compound 4:

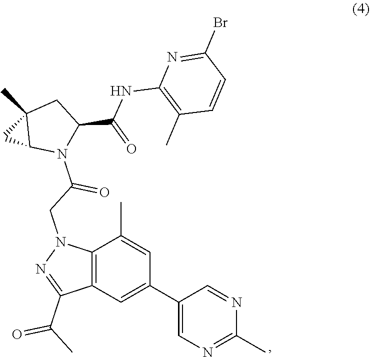

or a pharmaceutically acceptable salt thereof.
Embodiment 7. The method of any one of embodiments 1-6, wherein the betacoronavirus is at least one betacoronavirus selected from SARs-CoV, MERS-CoV, and SARs-CoV-2.
Embodiment 8. The method of any one of embodiments 1-7, wherein the betacoronavirus is SARs-CoV-2.
Embodiment 9. The method of any one of embodiments 1-8, wherein the human subject is exhibiting one or more respiratory symptoms.
Embodiment 10. The method of embodiment 9, wherein the respiratory symptom is at least one symptom selected from inflammation of cells in the large airway and parenchyma, perivascular cuffing, thickening of the interstitial membrane, intra-alveolar edema, rhinorrhea, sneezing, sore throat, pneumonia, lung ground-glass opacity, RNAemia, and acute respiratory distress syndrome (ARDS).
Embodiment 11. The method of any one of embodiments 1-10, wherein the human subject is exhibiting one or more systemic disorders.
Embodiment 12. The method of embodiment 11, wherein the systemic disorder is selected from acute cardiac injury, coughing, cytokine storm, nausea, vomiting, diarrhea, dyspnea, anosmia, fatigue, fever, headache, muscle or body ache, hemoptysis, hypoxemia, lymphopenia, renal injury, stroke, septic shock, sputum production, nasal congestion, refractory thrombosis, antiphospholipid syndrome, seizure, renal failure, and myocardial infarction.
Embodiment 13. The method of any one of embodiments 1-12, wherein the human subject is suffering from critical viral disease, wherein the critical viral disease comprises severe shortness of breath.
Embodiment 14. The method of any one of claims 1-13, wherein the human subject is suffering from critical viral disease, wherein the critical viral disease comprises at least one of acute lung injury (ALI), acute kidney injury (AKI), stroke, ARDS, failure of non-respiratory organs, respiratory failure, respiratory shock, and severe pneumonia.
Embodiment 15. The method of embodiment 14, wherein the human subject suffering from critical viral disease displays at least one symptom selected from progressive increase of peripheral inflammatory cytokines, progressive increase of lactate, progressive reduction of peripheral blood lymphocytes, and rapid progression of one or more lung pathologies.
Embodiment 16. The method of any one of embodiments 1-15, wherein the risk or duration of hospitalization of the human subject is reduced.
Embodiment 17. The method of any one of embodiments 1-16, wherein the risk of developing one or more respiratory syndromes in the human subject is reduced.
Embodiment 18. The method of embodiment 17, where the respiratory syndrome is selected from at least one of acute respiratory distress syndrome (ARDS), lung ground-glass opacity, inflammation of cells in the large airway and parenchyma, intra-alveolar edema, perivascular cuffing, pneumonia, rhinorrhea, RNAemia, sneezing, sore throat, and thickening of the interstitial membrane.
Embodiment 19. The method of any one of embodiments 1-18, wherein the risk of developing one or more systemic disorders in the human subject is reduced.
Embodiment 20. The method of embodiment 19, wherein the systemic disorder is selected from acute cardiac injury, coughing, cytokine storm, nausea, vomiting, diarrhea, dyspnea, anosmia, fatigue, fever, headache, muscle or body ache, hemoptysis, hypoxemia, lymphopenia, renal injury, stroke, septic shock, sputum production, nasal congestion, refractory thrombosis, antiphospholipid syndrome, seizure, renal failure, and myocardial infarction.
Embodiment 21. The method of embodiment 20, wherein the systemic disorder is renal failure.
Embodiment 22. The method of any one of embodiments 1-21, wherein the risk of developing a critical viral disease is reduced, and the critical viral disease comprises severe shortness of breath.
Embodiment 23. The method of any one of embodiments 1-22, wherein the risk of developing a critical viral disease is reduced, and the critical viral disease comprises at least one of ALI, AKI, stroke, ARDS, failure of non-respiratory organs, respiratory failure, respiratory shock, and severe pneumonia.
Embodiment 24. The method of any one of embodiments 1-23, wherein the risk of death of the human subject is reduced.
Embodiment 25. The method of any one of embodiments 1-24, wherein the risk or duration of need for non-invasive mechanical ventilation is reduced.
Embodiment 26. The method of any one of embodiments 1-25, wherein risk or duration of need for extracorporeal membrane oxygenation is reduced.
Embodiment 27. The method of any one of embodiments 1-26, wherein risk or duration of need for invasive mechanical ventilation is reduced.
Embodiment 28. The method of any one of embodiments 1-27, wherein risk or duration of need for high-flow oxygen therapy is reduced.
Embodiment 29. The method of any one of embodiments 1-28, wherein the risk or duration of need for respiratory intubation of the human subject is reduced.
Embodiment 30. The method of any one of embodiments 1-29, wherein the at least one CFD inhibitor is administered orally.
Embodiment 31. The method of embodiment 30, wherein the at least one CFD inhibitor is provided in a powder-filled capsule dosage form.
Embodiment 32. The method of embodiment 30, wherein the at least one CFD inhibitor is provided in tablet dosage form.
Embodiment 33. The method of embodiment 30, wherein the at least one CFD inhibitor is provided in a liquid-filled capsule dosage form.
Embodiment 34. The method of embodiment 30, wherein the at least one CFD inhibitor is provided in a gel-based dosage form.
Embodiment 35. The method of any one of embodiments 1-29, wherein the at least one CFD inhibitor is administered via a nasogastric tube.
Embodiment 36. The method of embodiment 35, wherein the at least one CFD inhibitor is administered as a solution or suspension.
Embodiment 37. The method of embodiment 36, wherein the solution or suspension is formed by providing the at least one CFD inhibitor in the form of particles and adding water to the particles.
Embodiment 38. The method of any one of embodiments 1-37, wherein said administering comprises administering to the human subject a loading dose of the at least one CFD inhibitor followed by a maintenance dose of the at least one factor D inhibitor.
Embodiment 39. The method of embodiment 38, wherein the maintenance dose comprises about 600 mg to about 1200 mg of the at least one CFD inhibitor administered in one or more doses per day.
Embodiment 40. The method of embodiment 39, wherein the maintenance dose comprises about 800 mg to about 1000 mg of the at least one CFD inhibitor administered in one or more doses per day.
Embodiment 41. The method of embodiment 39 or 40, wherein the maintenance dose is administered in four doses per day.
Embodiment 42. The method of embodiment 41, wherein each of the four doses comprises about 150 mg to about 300 mg dose of the at least one CFD inhibitor.
Embodiment 43. The method of embodiment 42, wherein each of the four doses comprises about 200 mg dose to about 250 mg of the at least one CFD inhibitor.
Embodiment 44. The method of any one of embodiments 38-43, wherein the loading dose comprises about 200 mg to about 500 mg of the at least one CFD inhibitor.
Embodiment 45. The method of embodiment 44, wherein the loading dose comprises about 300 mg to about 400 mg of the at least one CFD inhibitor.
Embodiment 46. The method of any one of embodiments 38-45, wherein the human subject is less than 70 years of age.
Embodiment 47. The method of embodiment 46, wherein the human subject is less than 50 years of age.
Embodiment 48. The method of embodiment 47, wherein the human subject is less than 18 years of age.
Embodiment 49. The method of any one of embodiments 48-48, wherein said administering comprises administering to the human subject a loading dose of about 400 mg of the at least one CFD inhibitor followed by a maintenance dose of the at least one CFD inhibitor, wherein the maintenance dose comprises about 250 mg of the at least one CFD inhibitor administered four times a day.
Embodiment 50. The method of any one of embodiments 38-45, wherein the human subject is at least 70 years of age.
Embodiment 51. The method of embodiment 50, wherein said administering comprises administering to the human subject a loading dose of about 300 mg of the at least one CFD inhibitor followed by a maintaining dose of the at least one CFD inhibitor, wherein the maintenance dose comprises about 200 mg of the at least one CFD inhibitor administered four times a day.
Embodiment 52. The method of any one of embodiments 1-51, wherein a Cₘₐₓ of the at least one CFD inhibitor of at least about 150 ng/mL, at least about 300 ng/mL, or from about 150 ng/mL to about 300 ng/mL, is provided.
Embodiment 53. The method of any one of embodiments 1-52, wherein a Cₘₐₓ of the at least one CFD inhibitor of at least about 235 ng/mL is provided.
Embodiment 54. The method of any one of embodiments 1-53, wherein a Cₘₐₓ of the at least one CFD inhibitor of less than about 1000 ng/mL is provided.
Embodiment 55. The method of any one of embodiments 1-54, wherein a Cₘₐₓ of the at least one CFD inhibitor of less than about 500 ng/mL is provided.
Embodiment 56. The method of any one of embodiments 1-55, further comprising determining Bb level elevation in the human subject prior to administering the at least one CFD inhibitor.
Embodiment 57. The method of any one of embodiments 1-56, wherein the human subject has at least one pre-existing condition that increases their risk of one or more of pneumonia, acute respiratory distress syndrome, respiratory failure, septic shock, organ failure, cytokine storm, or death.
Embodiment 58. The method of embodiment 57, wherein the pre-existing condition comprises at least one condition selected from cardiovascular disease, chronic respiratory disease, diabetes, hypertension, immune deficiency, and obesity.
Embodiment 59. The method of any one of embodiments 1-58, wherein the at least one CFD inhibitor is administered in combination with a second therapeutic agent.
Embodiment 60. The method of embodiment 59, wherein the at least one CFD inhibitor is administered in combination with remdesivir or a glycosaminoglycan.
Embodiment 61. The method of embodiment 59, wherein the at least one CFD inhibitor is administered in combination with remdesivir.
Embodiment 62. The method of embodiment 61, wherein remdesivir is administered as a 200-mg intravenous loading dose, followed by a 100-mg once-daily intravenous maintenance dose.
Embodiment 63. The method of embodiment 61, wherein the at least one CFD inhibitor is administered in combination with a glycosaminoglycan.
Embodiment 64. The method of embodiment 63, wherein the glycosaminoglycan is heparan sulfate, an α2,3- and α2,6-sialylated N-glycan, or an analog thereof.
Embodiment 65. The method of embodiment 64, wherein the glycosaminoglycan is a ReGeneraTing Agent or PI-88.
Embodiment 66. The method of any one of embodiments 1-65, wherein the human subject does not have an estimated glomerular filtration rate (eGFR) <30 mL/min.
Embodiment 67. The method of any one of embodiments 1-66, wherein the human subject has not received five or more doses of remdesivir prior to the start of treatment.
Embodiment 68. The method of any one of embodiments 1-67, wherein the human subject has not received two or more doses of >60 mg prednisone or an equivalent thereof in the 7 days prior to the start of treatment.
Embodiment 69. The method of any one of embodiments 1-68, wherein the human subject has not received a small molecule tyrosine kinase inhibitor in the 4 weeks prior to the start of treatment.
Embodiment 70. The method of any one of embodiments 1-89, wherein the human subject has not received a monoclonal antibody targeting a cytokine in the 4 weeks prior to the start of treatment.
Embodiment 71. The method of any one of embodiments 1-70, wherein the human subject has not received a monoclonal antibody targeting T-cells in the 4 weeks prior to the start of treatment.
Embodiment 72. The method of any one of embodiments 1-71, wherein the human subject has not received a monoclonal antibody targeting B-cells in the 3 months prior to the start of treatment.
Embodiment 73. The method of any one of embodiments 1-72, wherein the human subject has not received a granulocyte-macrophage colony-stimulating factor (GM-CSF) agent within 2 months prior to the start of treatment.
Embodiment 74. The method of any one of embodiments 1-73, wherein the human subject has not received an immunosuppressant in the 4 weeks prior to the start of treatment.
Embodiment 75. The method of any one of embodiments 1-74, wherein the human subject has not received a live vaccine prior to the start of treatment.
Embodiment 76. The method of any one of embodiments 1-75, wherein the human subject does not have active tuberculosis.
Embodiment 77. The method of any one of embodiments 1-76, wherein the human subject does not have a known history of a human immunodeficiency virus (HIV) infection.
Embodiment 78. The method of any one of embodiments 1-77, wherein the human subject does not have a known history of a hepatitis B virus (HBV) infection.
Embodiment 79. The method of any one of embodiments 1-78, wherein the human subject does not have a known history of a hepatitis C virus (HCV) infection.
Embodiment 80. The method of any one of embodiments 1-79, wherein the human subject does not have a known history of pulmonary alveolar proteinosis (PAP).
Embodiment 81. The method of any one of embodiments 1-80, wherein the human subject does not have an active malignancy.
Embodiment 82. The method of any one of embodiments 1-81, wherein the human subject does not have immunodeficiency.
Embodiment 83. The method of any one of embodiments 1-82, wherein the human subject does not have an uncontrolled opportunistic infection.
Embodiment 84. The method of any one of embodiments 1-83, wherein the human subject does not have uncontrolled cirrhosis.
Embodiment 85. The method of any one of embodiments 1-84, wherein the human subject is not tested positive for an influenza virus.
Embodiment 86. The method of any one of embodiments 1-85, wherein the human subject is prohibited from receiving a small molecule tyrosine kinase inhibitor.
Embodiment 87. The method of any one of embodiments 1-86, wherein the human subject is prohibited from receiving a monoclonal antibody targeting a cytokine.
Embodiment 88. The method of any one of embodiments 1-87, wherein the human subject is prohibited from receiving a monoclonal antibody targeting T-cells.
Embodiment 89. The method of any one of embodiments 1-88, wherein the human subject is prohibited from receiving a monoclonal antibody targeting B-cells.
Embodiment 90. The method of any one of embodiments 1-89, wherein the human subject is prohibited from receiving an immunosuppressant.
Embodiment 91. The method of any one of embodiments 1-90, wherein the pat human subject lent is prohibited from receiving chloroquine or hydroxychloroquine.
Embodiment 92. The method of any one of embodiments 1-91, wherein the human subject has activated pathway of complement (APC).
Embodiment 93. A CFD inhibitor for use in a method of treating a betacoronavirus infection in a human subject, wherein the method is a method of any one of embodiments 1-90.
Embodiment 94. Use of a CFD inhibitor in the manufacture of a medicament for use in a method of treating a betacoronavirus infection in a human subject, wherein the method is a method of any one of embodiments 1-92.
Embodiment 95. A method for diagnosing a SARS-CoV-2 infection (e.g., COVID-19) in a patient having or suspected of having a SARS-CoV-2 infection (COVID-19), comprising (a) incubating serum obtained from the patient and, optionally, a control serum with a plurality of glycosylphosphatidylinositol-anchored protein (GPI-AP) deficient cells; (b) measuring cell viability of GPI-AP deficient cells in the incubate of (a); (c) comparing the cell viability of the GPI-AP deficient cells incubated with the patient's serum with (1) a reference standard or (2) the viability of GPI-AP deficient cells incubated with the control serum, wherein a reduction in the cell viability in the patient serum incubate compared to the reference standard or the control serum incubate is indicative that the patient has or is likely to have SARS-CoV-2 infection (e.g., COVID-19).
Embodiment 96. A method for diagnosing activated alternate pathway of complement (APC) in a patient with a SARS-CoV-2 infection (e.g., COVID-19), comprising (a) incubating serum obtained from the patient and, optionally, a control serum with a plurality of GPI-AP deficient cells; (b) measuring cell viability of GPI-AP deficient cells in the incubate of (a); (c) comparing the cell viability of GPI-AP deficient cells incubated with the patient's serum with (1) a reference standard or (2) the viability of GPI-AP deficient cells incubated with the control serum, wherein a reduction in the cell viability in the patient serum incubate compared to the reference standard or the control serum incubate is indicative that the patient with a SARS-CoV-2 infection (e.g., COVID-19) has activated APC.
Embodiment 97. A method for classifying a patient with a SARS-CoV-2 infection (e.g., COVID-19) based on activated APC status, comprising: (a) diagnosing APC in a patient with a SARS-CoV-2 infection (e.g., COVID-19) according to the method of embodiment 96; and (b) classifying the patient as having activated APC (APC+ infection) if a reduction in the cell viability of the GPI-AP deficient cells in the patient serum incubate compared the cell viability of the GPI-AP deficient cells in the reference standard or the control serum incubate is observed; or classifying the patient as having infection with baseline APC if the cell viability of the GPI-AP cells in the patient serum incubate is unchanged or elevated compared to the cell viability of the GPI-AP cells in the reference standard or the control serum incubate.
Embodiment 98. A method for treating a SARS-CoV-2 infection (e.g., COVID-19) in a patient with activated APC, comprising, classifying a patient according to the method of claim 97 and, if the patient is classified as having activated APC, administering to the patient a therapeutically effective amount of a complement modulator.
Embodiment 99. The method of embodiment 98, wherein the complement modulator is (a) a CFD inhibitor; (b) a complement C5 inhibitor; (c) a complement C3 inhibitor; (d) a combination of a complement C3 inhibitor and a complement C5 inhibitor; (e) a Factor H (FH) protein; or (f) a Factor B (FB) inhibitor.
Embodiment 100. The method of embodiment 99, wherein the complement modulator is a CFD inhibitor.
Embodiment 101. The method of embodiment 100, wherein the complement modulator is the CFD inhibitor for use of embodiment 94.
Embodiment 102. The method of any one of embodiments 95-101, wherein the patient has COVID-19.
Embodiment 103. The method of any one of embodiments 95-102, wherein the method comprises incubating a control serum with a plurality of GPI-AP deficient cells.
Embodiment 104. The method of embodiment 101, wherein the control serum is a serum obtained from a healthy subject.
Embodiment 105. The method of embodiment 104, wherein the healthy subject is a subject who has never had a SARS-CoV-2 infection (e.g., COVID-19).
Embodiment 106. The method of embodiment 104, wherein the healthy subject is a subject who has fully recovered from a SARS-CoV-2-infection.
Embodiment 107. The method of any one of embodiments 103-105, wherein the control serum is supplemented with SARS-CoV-2 N protein and/or a spike protein from a benign human coronavirus (e.g., HCoV-OC43).
Embodiment 108. The method of any one of embodiments 95-102, wherein the cell viability of the GPI-AP deficient cells incubated with the patient's serum is compared with a reference standard, wherein the reference standard is a baseline cell viability of GPI-AP deficient cells in healthy human serum.
Embodiment 109. The method of embodiment 108, wherein the baseline cell viability is at least 70% (e.g., at least 80%, at least 90%, at least 95%, or at least 99%).
Embodiment 110. The method of any one of embodiments 95-109, further comprising (3) measuring deposition of a complement protein on the surface of the GPI-AP deficient cells, wherein the complement protein is selected from the group of C5b9, C4d, C3c, and any combination thereof.
Embodiment 111. The method of embodiment 110, wherein the deposition of the complement protein is measured using a fluorescence assay, e.g., fluorescence activated cell sorter (FACS).
Embodiment 112. The method of any one of embodiments 95-111, wherein cell viability is measured using a fluorescence assay, e.g., FACS.
Embodiment 113. The method of any one of embodiments 95-112, wherein the GPI-AP deficient cells are PIGA null blood cells.
Embodiment 114. The method of embodiment 113, wherein the GPI-AP deficient cells are PIGA null lymphoblast TF1 (TF1PIGAnull) cells.
Embodiment 115. A method of assessing a risk of a patient with SARS-CoV-2 infection (e.g., COVID-19) to develop a severe vascular complication (e.g., disseminated thrombosis and/or multi-organ failure), comprising: (a) detecting the presence or absence of one or more mutations in complement proteins in a cell sample (e.g., a blood cell sample) obtained from the patient; and (b) identifying the patient as being at high risk for developing the vascular complication if the patient's cell sample comprises one or more mutations in complement proteins.
Embodiment 116. The method of embodiment 115, wherein the one or more mutations in complement proteins comprises a gain of function (GOF) mutation in a complement activating factor and/or a loss of function (LOF) mutation in a complement inhibitory factor.
Embodiment 117. The method of embodiment 116, wherein the GOF mutation in a complement activating factor is a GOF mutation in complement C3 and/or complement factor B; and the LOF mutation in a complement inhibitory factor is a LOF mutation in one or more of complement factor H (CFH); a CFH-related protein selected from complement factor H receptor 1 (CFHR1), complement factor H receptor 2 (CFHR2), complement factor H receptor 3 (CFHR3), complement factor H receptor 1 (CFHR4), and complement factor H receptor 5 (CFHR5); complement factor I (CFI); membrane cofactor protein (MCP or CD46); thrombomodulin (THBD); and complement receptor 1 (CR1).
Embodiment 118. A method for treating a patient with a SARS-CoV-2 infection (e.g., COVID-19) who is at risk for developing a severe vascular complication (e.g., disseminated thrombosis and/or multi-organ failure), comprising: (a) identifying a patient as being at high risk for developing the vascular complication according to the method of any one of embodiments 115-117; and (b) administering the patient a therapeutically effective amount of a complement modulator.
Embodiment 119. The method of embodiment 118, wherein the complement modulator is (a) a CFD inhibitor; (b) a complement C5 inhibitor; (c) a complement C3 inhibitor; (d) a combination of a complement C3 inhibitor and a complement C5 inhibitor; (e) a Factor H (FH) protein; or (f) a Factor B (FB) inhibitor.
Embodiment 120. The method of embodiment 119, wherein the complement modulator is a CFD inhibitor.
Embodiment 121. The method of embodiment 119, wherein the complement modulator is the CFD inhibitor for use of embodiment 94.
Embodiment 122. A method of screening for test agents that inhibit SARS-CoV-2 spike protein-mediated cell death, comprising: (a) incubating GPI-AP deficient cells with human serum comprising recombinant SARS-CoV-2 spike proteins S1 and/or S2 for a period sufficient to activate APC on the GPI-AP cells to form an incubate; (b) treating the incubate: (i) with a test agent to produce a test incubate; and (ii) without a test agent to produce a null incubate; (c) measuring cell viability of the GPI-AP deficient cells in the test and null incubates; and (d) comparing the cell viability of the GPI-AP deficient cells in the test incubate to the cell viability in the null incubate; and wherein an increase in the cell viability in the test incubate compared to the cell viability in the null incubate indicates that the test agent is capable of inhibiting SARS-CoV-2 spike protein-mediated cell death cell death.
Embodiment 123. The method of embodiment 122, wherein the one or more control agents comprises an agent which effectively blocks the binding of SARS-CoV-2 spike proteins to cell surface.
Embodiment 124. The method of embodiment 123, wherein the agent which effectively blocks the binding of SARS-CoV-2 spike proteins to cell surface is heparan sulfate or a derivative thereof.
Embodiment 125. The method of any one of embodiments 122-124, further comprising determining deposition of complement proteins C5b9, C4d, and/or C3c on the surface of GPI-AP deficient cells in the null sample and the test sample.
Embodiment 126. The method of any one of embodiments 122-125, wherein step (b) further comprises (iii) treating the incubate with one or more control agents to produce one or more control incubates, and wherein the method further comprises (e) comparing the cell viability of GPI-AP deficient cells in the test incubate to the cell viability of the GPI-AP deficient cells in the one or more control incubates, wherein an increase in the cell viability in the test incubate compared to the cell viability in the one or more control incubates indicates that the test agent is capable of inhibiting SARS-CoV-2 spike-protein-mediated cell death.
Embodiment 127. The method of embodiment 126, wherein the cell viability of GPI-AP cells in the one or more control incubates is higher than that of GPI-AP cells in the null incubate.
Embodiment 128. The method of embodiment 126 or 127, further comprising determining deposition of complement proteins C5b9, C4d, and/or C3c on the surface of GPI-AP deficient cells in the one or more control samples.
Embodiment 129. The method of embodiment 125 or 127, wherein the deposition of complement proteins is measured using a fluorescence assay, e.g., FACS.
Embodiment 130. The method of any one of embodiments 122-129, wherein cell viability is measured using a fluorescence assay, e.g., FACS.
Embodiment 131. The method of any one of embodiments 122-130, wherein the GPI-AP deficient cells are PIGA null blood cells.
Embodiment 132. The method of embodiment 131, wherein the GPI-AP deficient cells are TF1PIGAnull cells.
Embodiment 133. A method comprising the steps of a method comprising the steps of (a) incubating serum obtained from a patient having or suspected of having COVID-19 with a plurality of GPI-AP deficient cells; and (b) performing a cell viability assay on the cells from step (a).
Embodiment 134. The method of embodiment 133, further comprising the step of diagnosing the patient as having an activated APC based on a statistically significant increased difference of non-viable cells from the patient's serum as compared to a control.
Embodiment 135. A method for diagnosing activated APC in a COVID-19 patient comprising the steps of (a) incubating serum obtained from a patient having COVID-19 with a plurality of GPI-AP deficient cells; (b) performing a cell viability assay on the cells from step (a); and (c) diagnosing the patient as having activated APC based on a statistically significant increased difference of non-viable cells from the patient's serum as compared to a control.
Embodiment 136. A method for treating activated APC in a COVID-19 patient comprising the steps of administering an effective amount of a CFD inhibitor to a COVID-19 patient diagnosed with activated APC according to the method of embodiment 135.
Embodiment 137. A method for treating activated APC in a COVID-19 patient comprising the steps of administering an effective amount of a C5 inhibitor and a C3 inhibitor to a COVID-19 patient diagnosed with activated APC according to the method of embodiment 135.
Embodiment 138. A method for treating activated APC in a COVID-19 patient comprising the step of administering an effective amount of a CFD inhibitor to a patient diagnosed with activated APC based on the performance of a cell viability assay on a plurality of GPI-AP deficient cells that have been incubated with serum obtained from the patient, wherein the diagnosis is based on a statistically significant increased difference of non-viable cells from the patient's serum as compared to a control.
Embodiment 139. A method for treating activated APC in a COVID-19 patient comprising the step of administering an effective amount of a C5 inhibitor and an effective amount of a C3 inhibitor to a patient diagnosed with activated APC based on the performance of a cell viability assay on a plurality of GPI-AP deficient cells that have been incubated with serum obtained from the patient, wherein the diagnosis is based on a statistically significant increased difference of non-viable cells from the patient's serum as compared to a control.
Embodiment 140. A method for treating activated APC in a COVID-19 patient comprising the steps of (a) incubating serum obtained from a COVID-19 patient with a plurality of GPI-AP deficient cells; (b) performing a cell viability assay on the cells from step (a); (c) diagnosing the patient as having activated APC based on a statistically significant increased difference of non-viable cells from the patient's serum as compared to a control; and (d) administering an effective amount of a CFD inhibitor to the patient.
Embodiment 141. A method for treating activated APC in a COVID-19 patient comprising the steps of: (a) incubating serum obtained from a COVID-19 patient with a plurality of GPI-AP deficient cells; (b) performing a cell viability assay on the cells from step (a); (c) diagnosing the patient as having activated APC based on a statistically significant increased difference of non-viable cells from the patient's serum as compared to a control; and (d) administering an effective amount of a C5 inhibitor and a C3 inhibitor to the patient.
Embodiment 142. The method of any one of embodiments 133-141, wherein the plurality of GPI-AP deficient cells is biochemically treated to remove GPI-AP.
Embodiment 143. The method of any one of embodiments 133-142, wherein the plurality of GPI-AP deficient cells is PIGA null mutant cell line.
Embodiment 144. The method of any one of embodiments 133-143, wherein the cell viability assay is a WST-1 cell viability assay.
Embodiment 145. The method of any one of embodiments 136, 138, 140, and 142-144, wherein the CFD inhibitor is Compound 1:

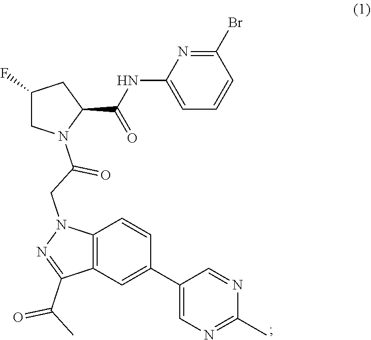 [SMILES: CC(=O)c1nn(CC(=O)N2C[C@H](F)C[C@H]2C(=O)Nc2cccc(Br)n2)c2ccc(-c3cnc(C)nc3)cc12]

Compound (3):

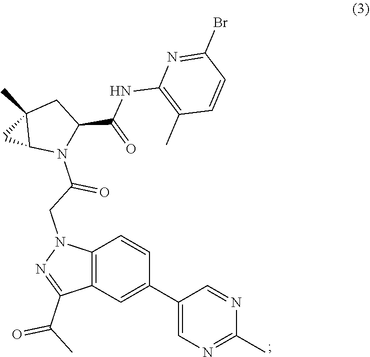

or Compound (4):

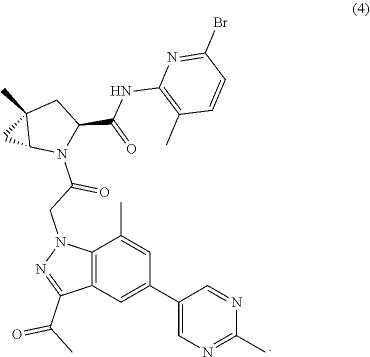

Embodiment 146. The method of any one of embodiments 137, 139, and 140-144, wherein the C5 inhibitor is eculizumab, ravulizumab, coversin, cemdisiran, LFG-316, SOB31005, SKY59, REGN3918, TNX-558, neutrazumab, CCX168, ABP959, GNR-45, zimura, RA101495, ISU305, mubodina, IFX-1, ALS-205, DF2593A, or IPH5401.
Embodiment 147. The method of any one of embodiments 137, 139, 140-144, and 146, wherein the C3 inhibitor is AMY-101, APL-1, APL-2, or APL-9.
Embodiment 148. The method of any one of embodiments 136-147, wherein the patient is further treated with Factor H.
Embodiment 149. A method for treating activated APC in a COVID-19 patient comprising the steps of administering an effective amount of Factor H to a COVID-19 patient diagnosed with activated APC according to the method of embodiment 135.
Embodiment 150. A method for treating activated APC in a COVID-19 patient comprising the step of administering an effective amount of Factor H to a patient diagnosed with activated APC based on the performance of a cell viability assay on a plurality of GPI-AP deficient cells that have been incubated with serum obtained from the patient, wherein the diagnosis is based on a statistically significant increased difference of non-viable cells from the patient serum as compared to a control.
Embodiment 151. A method for treating activated APC in a COVID-19 patient comprising the steps of (a) incubating serum obtained from a COVID-19 patient with a plurality of GPI-AP deficient cells; (b) performing a cell viability assay on the cells from step (a); (c) diagnosing the patient as having activated APC based on a statistically significant increased difference of non-viable cells from the patient serum as compared to a control; and (d) administering an effective amount of Factor H inhibitor to the patient.
Embodiment 152. The method of any one of embodiments 149-151, wherein the plurality of GPI-AP deficient cells is a PIGA null mutant cell line.
Embodiment 153. The method of any one of embodiments 149-152 wherein the cell viability assay is the WST-1 cell viability assay.
Embodiment 154. The method of any one of embodiments 149-153, wherein the patient is treated with either a CFD inhibitor or a combination of a C5 inhibitor and a C3 inhibitor.
Embodiment 155. The method of any one of embodiments 133-154, wherein the COVID-19 patient is tested for mutations in a complement-related gene.
Embodiment 156. The method of embodiment 155, wherein the mutation comprises a loss of function mutation in a complement inhibitory factor or a gain of function mutation of a complement activating factor.
Embodiment 157. The method of embodiment 156, where in the complement inhibitory factor is complement factor H (CFH), complement factor I (CFI), CD46, thrombomodulin (THBD), or complement receptor 1 (CR1).
Embodiment 158. The method of embodiment 156 or 157, wherein the complement activating factor is complement factor B (CFB) or complement component C3.

### Other Embodiments

While the disclosure describes specific embodiments of methods, compounds, and uses, it will be understood that further modifications can be made thereto, and this application is intended to cover any variations or adaptations thereof following, in general, the principles of the disclosure including such departures from the disclosure that come within known or customary practice within the art to which the disclosure pertains and may be applied to essential features hereinbefore set forth, and follows in the scope of the claims. Other embodiments are within the claims.

What is claimed is:

1. A method of treating a betacoronavirus infection in a human subject, the method comprising administering to the human subject a therapeutically effective amount of at least one complement factor D (CFD) inhibitor.

2. The method of claim 1, wherein the at least one CFD inhibitor is selected from Compound 1, Compound 2, Compound 3, and Compound 4: 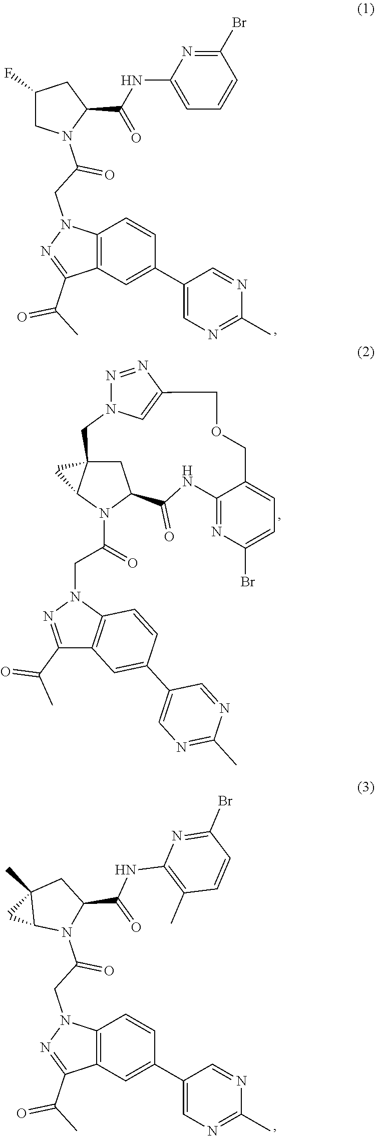 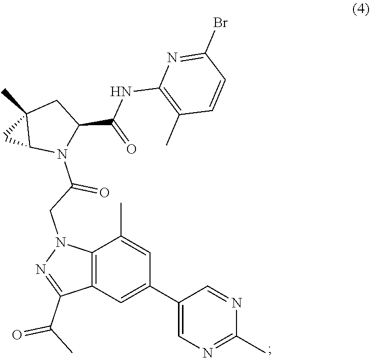
and pharmaceutically acceptable salts thereof.

3. The method of claim 2, wherein the at least one CFD inhibitor is Compound 1: 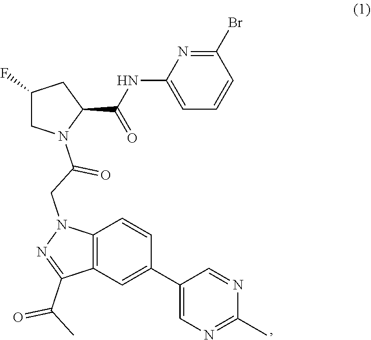 [SMILES: CC(=O)c1nn(CC(=O)N2C[C@H](F)C[C@H]2C(=O)Nc2cccc(Br)n2)c2ccc(-c3cnc(C)nc3)cc12]
or a pharmaceutically acceptable salt thereof.

4. The method of claim 2, wherein the at least one CFD inhibitor is Compound 2: 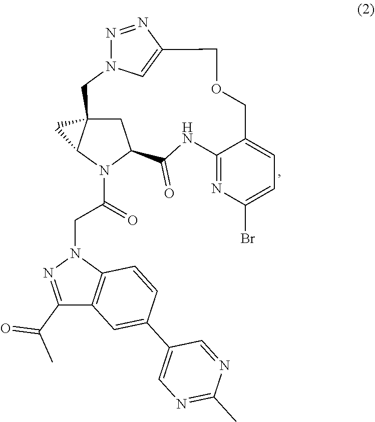 [SMILES: CC(=O)c1nn(CC(=O)N2[C@H]3C[C@]4(C[C@@H]24)Cn2cc(nn2)COCc2ccc(Br)nc2NC3=O)c2ccc(-c3cnc(C)nc3)cc12]
or a pharmaceutically acceptable salt thereof.

5. The method of claim 2, wherein the at least one CFD inhibitor is Compound 3: 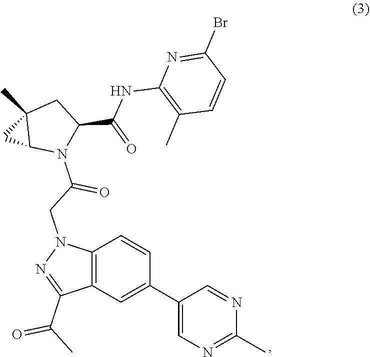
or a pharmaceutically acceptable salt thereof.

6. The method of claim 2, wherein the at least one CFD inhibitor is Compound 4: 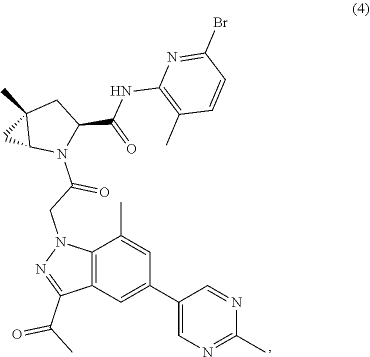
or a pharmaceutically acceptable salt thereof.

7. The method of any of claims 1-6, wherein the betacoronavirus is at least one betacoronavirus selected from SARs-CoV, MERS-CoV, and SARs-CoV-2.

8. The method of any one of claims 1-6, wherein the betacoronavirus is SARs-CoV-2.

9. The method of any one of claims 1-8, wherein the human subject is exhibiting one or more respiratory symptoms.

10. The method of claim 9, wherein the respiratory symptom is at least one symptom selected from inflammation of cells in the large airway and parenchyma, perivascular cuffing, thickening of the interstitial membrane, intra-alveolar edema, rhinorrhea, sneezing, sore throat, pneumonia, lung ground-glass opacity, RNAemia, and acute respiratory distress syndrome (ARDS).

11. The method of any one of claims 1-10, wherein the human subject is exhibiting one or more systemic disorders.

12. The method of claim 11, wherein the systemic disorder is selected from acute cardiac injury, coughing, cytokine storm, nausea, vomiting, diarrhea, dyspnea, anosmia, fatigue, fever, headache, muscle or body ache, hemoptysis, hypoxemia, lymphopenia, renal injury, stroke, septic shock, sputum production, nasal congestion, refractory thrombosis, antiphospholipid syndrome, seizure, renal failure, and myocardial infarction.

13. The method of any one of claims 1-12, wherein the human subject is suffering from critical viral disease, wherein the critical viral disease comprises severe shortness of breath.

14. The method of any one of claims 1-13, wherein the human subject is suffering from critical viral disease, wherein the critical viral disease comprises at least one of acute lung injury (ALI), acute kidney injury (AKI), stroke, ARDS, failure of non-respiratory organs, respiratory failure, respiratory shock, and severe pneumonia.

15. The method of claim 14, wherein the human subject suffering from critical viral disease displays at least one symptom selected from progressive increase of peripheral inflammatory cytokines, progressive increase of lactate, progressive reduction of peripheral blood lymphocytes, and rapid progression of one or more lung pathologies.

16. The method of any one of claims 1-15, wherein the risk or duration of hospitalization of the human subject is reduced.

17. The method of any one of claims 1-16, wherein the risk of developing one or more respiratory syndromes in the human subject is reduced.

18. The method of claim 17, where the respiratory syndrome is selected from at least one of acute respiratory distress syndrome (ARDS), lung ground-glass opacity, inflammation of cells in the large airway and parenchyma, intra-alveolar edema, perivascular cuffing, pneumonia, rhinorrhea, RNAemia, sneezing, sore throat, and thickening of the interstitial membrane.

19. The method of any one of claims 1-18, wherein the risk of developing one or more systemic disorders in the human subject is reduced.

20. The method of claim 19, wherein the systemic disorder is selected from acute cardiac injury, coughing, cytokine storm, nausea, vomiting, diarrhea, dyspnea, anosmia, fatigue, fever, headache, muscle or body ache, hemoptysis, hypoxemia, lymphopenia, renal injury, stroke, septic shock, sputum production, nasal congestion, refractory thrombosis, antiphospholipid syndrome, seizure, renal failure, and myocardial infarction.

21. The method of claim 20, wherein the systemic disorder is renal failure.

22. The method of any one of claims 1-21, wherein the risk of developing a critical viral disease is reduced, and the critical viral disease comprises severe shortness of breath.

23. The method of any one of claims 1-22, wherein the risk of developing a critical viral disease is reduced, and the critical viral disease comprises at least one of ALI, AKI, stroke, ARDS, failure of non-respiratory organs, respiratory failure, respiratory shock, and severe pneumonia.

24. The method of any one of claims 1-23, wherein the risk of death of the human subject is reduced.

25. The method of any one of claims 1-24, wherein the risk or duration of need for non-invasive mechanical ventilation is reduced.

26. The method of any one of claims 1-25, wherein risk or duration of need for extracorporeal membrane oxygenation is reduced.

27. The method of any one of claims 1-26, wherein risk or duration of need for invasive mechanical ventilation is reduced.

28. The method of any one of claims 1-27, wherein risk or duration of need for high-flow oxygen therapy is reduced.

29. The method of any one of claims 1-28, wherein the risk or duration of need for respiratory intubation of the human subject is reduced.

30. The method of any one of claims 1-29, wherein the at least one CFD inhibitor is administered orally.

31. The method of claim 30, wherein the at least one CFD inhibitor is provided in a powder-filled capsule dosage form.

32. The method of claim 30, wherein the at least one CFD inhibitor is provided in tablet dosage form.

33. The method of claim 30, wherein the at least one CFD inhibitor is provided in a liquid-filled capsule dosage form.

34. The method of claim 30, wherein the at least one CFD inhibitor is provided in a gel-based dosage form.

35. The method of any one of claims 1-29, wherein the at least one CFD inhibitor is administered via a nasogastric tube.

36. The method of claim 35, wherein the at least one CFD inhibitor is administered as a solution or suspension.

37. The method of claim 36, wherein the solution or suspension is formed by providing the at least one CFD inhibitor in the form of particles and adding water to the particles.

38. The method of any one of claims 1-37, wherein said administering comprises administering to the human subject a loading dose of the at least one CFD inhibitor followed by a maintenance dose of the at least one factor D inhibitor.

39. The method of claim 38, wherein the maintenance dose comprises about 600 mg to about 1200 mg of the at least one CFD inhibitor administered in one or more doses per day.

40. The method of claim 39, wherein the maintenance dose comprises about 800 mg to about 1000 mg of the at least one CFD inhibitor administered in one or more doses per day.

41. The method of claim 39 or 40, wherein the maintenance dose is administered in four doses per day.

42. The method of claim 41, wherein each of the four doses comprises about 150 mg to about 300 mg dose of the at least one CFD inhibitor.

43. The method of claim 42, wherein each of the four doses comprises about 200 mg dose to about 250 mg of the at least one CFD inhibitor.

44. The method of any one of claims 38-43, wherein the loading dose comprises about 200 mg to about 500 mg of the at least one CFD inhibitor.

45. The method of claim 44, wherein the loading dose comprises about 300 mg to about 400 mg of the at least one CFD inhibitor.

46. The method of any one of claims 38-45, wherein the human subject is less than 70 years of age.

47. The method of claim 46, wherein the human subject is less than 50 years of age.

48. The method of claim 47, wherein the human subject is less than 18 years of age.

49. The method of any one of claims 48-48, wherein said administering comprises administering to the human subject a loading dose of about 400 mg of the at least one CFD inhibitor followed by a maintenance dose of the at least one CFD inhibitor, wherein the maintenance dose comprises about 250 mg of the at least one CFD inhibitor administered four times a day.

50. The method of any one of claims 38-45, wherein the human subject is at least 70 years of age.

51. The method of claim 50, wherein said administering comprises administering to the human subject a loading dose of about 300 mg of the at least one CFD inhibitor followed by a maintaining dose of the at least one CFD inhibitor, wherein the maintenance dose comprises about 200 mg of the at least one CFD inhibitor administered four times a day.

52. The method of any one of claims 1-51, wherein a C_{trough} of the at least one CFD inhibitor of at least about 150 ng/mL, at least about 300 ng/mL, or from about 150 ng/mL to about 300 ng/mL, is provided.

53. The method of any one of claims 1-52, wherein a C_{trough} of the at least one CFD inhibitor of at least about 235 ng/mL is provided.

54. The method of any one of claims 1-53, wherein a Cₘₐₓ of the at least one CFD inhibitor of less than about 1000 ng/mL is provided.

55. The method of any one of claims 1-54, wherein a Cₘₐₓ of the at least one CFD inhibitor of less than about 500 ng/mL is provided.

56. The method of any one claims 1-55, further comprising determining Bb level elevation in the human subject prior to administering the at least one CFD inhibitor.

57. The method of any one of claims 1-56, wherein the human subject has at least one pre-existing condition that increases their risk of one or more of pneumonia, acute respiratory distress syndrome, respiratory failure, septic shock, organ failure, cytokine storm, or death.

58. The method of claim 57, wherein the pre-existing condition comprises at least one condition selected from cardiovascular disease, chronic respiratory disease, diabetes, hypertension, immune deficiency, and obesity.

59. The method of any one of claims 1-58, wherein the at least one CFD inhibitor is administered in combination with a second therapeutic agent.

60. The method of claim 59, wherein the at least one CFD inhibitor is administered in combination with remdesivir or a glycosaminoglycan.

61. The method of claim 59, wherein the at least one CFD inhibitor is administered in combination with remdesivir.

62. The method of claim 61, wherein remdesivir is administered as a 200-mg intravenous loading dose, followed by a 100-mg once-daily intravenous maintenance dose.

63. The method of claim 61, wherein the at least one CFD inhibitor is administered in combination with a glycosaminoglycan.

64. The method of claim 63, wherein the glycosaminoglycan is heparan sulfate, an α2,3- and α2,6-sialylated N-glycan, or an analog thereof.

65. The method of claim 64, wherein the glycosaminoglycan is a ReGeneraTing Agent or PI-88.

66. The method of any one of claims 1-65, wherein the human subject does not have an estimated glomerular filtration rate (eGFR) <30 mL/min.

67. The method of any one of claims 1-66, wherein the human subject has not received five or more doses of remdesivir prior to the start of treatment.

68. The method of any one of claims 1-67, wherein the human subject has not received two or more doses of >60 mg prednisone or an equivalent thereof in the 7 days prior to the start of treatment.

69. The method of any one of claims 1-68, wherein the human subject has not received a small molecule tyrosine kinase inhibitor in the 4 weeks prior to the start of treatment.

70. The method of any one of claims 1-69, wherein the human subject has not received a monoclonal antibody targeting a cytokine in the 4 weeks prior to the start of treatment.

71. The method of any one of claims 1-70, wherein the human subject has not received a monoclonal antibody targeting T-cells in the 4 weeks prior to the start of treatment.

72. The method of any one of claims 1-71, wherein the human subject has not received a monoclonal antibody targeting B-cells in the 3 months prior to the start of treatment.

73. The method of any one of claims 1-72, wherein the human subject has not received a granulocyte-macrophage colony-stimulating factor (GM-CSF) agent within 2 months prior to the start of treatment.

74. The method of any one of claims 1-73, wherein the human subject has not received an immunosuppressant in the 4 weeks prior to the start of treatment.

75. The method of any one of claims 1-74, wherein the human subject has not received a live vaccine prior to the start of treatment.

76. The method of any one of claims 1-75, wherein the human subject does not have active tuberculosis.

77. The method of any one of claims 1-76, wherein the human subject does not have a known history of a human immunodeficiency virus (HIV) infection.

78. The method of any one of claims 1-77, wherein the human subject does not have a known history of a hepatitis B virus (HBV) infection.

79. The method of any one of claims 1-78, wherein the human subject does not have a known history of a hepatitis C virus (HCV) infection.

80. The method of any one of claims 1-79, wherein the human subject does not have a known history of pulmonary alveolar proteinosis (PAP).

81. The method of any one of claims 1-80, wherein the human subject does not have an active malignancy.

82. The method of any one of claims 1-81, wherein the human subject does not have immunodeficiency.

83. The method of any one of claims 1-82, wherein the human subject does not have an uncontrolled opportunistic infection.

84. The method of any one of claims 1-83, wherein the human subject does not have uncontrolled cirrhosis.

85. The method of any one of claims 1-84, wherein the human subject is not tested positive for an influenza virus.

86. The method of any one of claims 1-85, wherein the human subject is prohibited from receiving a small molecule tyrosine kinase inhibitor.

87. The method of any one of claims 1-86, wherein the human subject is prohibited from receiving a monoclonal antibody targeting a cytokine.

88. The method of any one of claims 1-87, wherein the human subject is prohibited from receiving a monoclonal antibody targeting T-cells.

89. The method of any one of claims 1-88, wherein the human subject is prohibited from receiving a monoclonal antibody targeting B-cells.

90. The method of any one of claims 1-89, wherein the human subject is prohibited from receiving an immunosuppressant.

91. The method of any one of claims 1-90, wherein the human subject is prohibited from receiving chloroquine or hydroxychloroquine.

92. The method of any one of claims 1-91, wherein the human subject has activated pathway of complement (APC).

93. A CFD inhibitor for use in a method of treating a betacoronavirus infection in a patient, wherein the method is a method of any one of claims 1-92.

94. Use of a CFD inhibitor in the manufacture of a medicament for use in a method of treating a betacoronavirus infection in a human subject, wherein the method is a method of any one of claims 1-92.

95. A method for diagnosing a SARS-CoV-2 infection in a patient having or suspected of having a SARS-CoV-2 infection, comprising (a) incubating serum obtained from the patient and, optionally, a control serum with a plurality of glycosylphosphatidylinositol-anchored protein (GPI-AP) deficient cells; (b) measuring cell viability of GPI-AP deficient cells in the incubate of (a); (c) comparing the cell viability of the GPI-AP deficient cells incubated with the patient's serum with (1) a reference standard or (2) the viability of GPI-AP deficient cells incubated with the control serum, wherein a reduction in the cell viability in the patient serum incubate compared to the reference standard or the control serum incubate is indicative that the patient has or is likely to have a SARS-CoV-2 infection.

96. A method for diagnosing activated APC in a patient with a SARS-CoV-2 infection, comprising (a) incubating serum obtained from the patient and, optionally, a control serum with a plurality of GPI-AP deficient cells; (b) measuring cell viability of GPI-AP deficient cells in the incubate of (a); (c) comparing the cell viability of GPI-AP deficient cells incubated with the patient's serum with (1) a reference standard or (2) the viability of GPI-AP deficient cells incubated with the control serum, wherein a reduction in the cell viability in the patient serum incubate compared to the reference standard or the control serum incubate is indicative that the patient with a SARS-CoV-2 infection has activated APC.

97. A method for classifying a patient with a SARS-CoV-2 infection based on activated APC status, comprising:
(a) diagnosing APC in a patient with a SARS-CoV-2 infection according to the method of claim 94; and
(b) classifying the patient as having activated APC if a reduction in the cell viability of the GPI-AP deficient cells in the patient serum incubate compared the cell viability of the GPI-AP deficient cells in the reference standard or the control serum incubate is observed; or
classifying the patient as having infection with baseline APC if the cell viability of the GPI-AP cells in the patient serum incubate is unchanged or elevated compared to the cell viability of the GPI-AP cells in the reference standard or the control serum incubate.

98. A method for treating a SARS-CoV-2 infection in a patient with activated APC, comprising, classifying a patient according to the method of claim 95 and, if the patient is classified as having activated APC, administering to the patient a therapeutically effective amount of the CFD inhibitor for use of claim 94.

99. The method of any one of claims 95-98, wherein the patient has COVID-19.

100. The method of any one of claims 95-99, wherein the method comprises incubating a control serum with a plurality of GPI-AP deficient cells.

101. The method of claim 100, wherein the control serum is a serum obtained from a healthy subject.

102. The method of claim 101, wherein the healthy subject is a subject who has never had a SARS-CoV-2 infection.

103. The method of claim 101, wherein the healthy subject is a subject who has fully recovered from a SARS-CoV-2 infection.

104. The method of any one of claims 101-103, wherein the control serum is supplemented with SARS-CoV-2 N protein and/or a spike protein from a benign human coronavirus.

105. The method of any one of claims 95-99, wherein the cell viability of the GPI-AP deficient cells incubated with the patient's serum is compared with a reference standard, wherein the reference standard is a baseline cell viability of GPI-AP deficient cells in healthy human serum.

106. The method of claim 105, wherein the baseline cell viability is at least 70%.

107. The method of any one of claims 95-106, further comprising (3) measuring deposition of a complement protein on the surface of the GPI-AP deficient cells, wherein the complement protein is selected from the group of C5b9, C4d, C3c, and any combination thereof.

108. The method of claim 107, wherein the deposition of the complement protein is measured using a fluorescence assay.

109. The method of any one of claims 95-108, wherein cell viability is measured using a fluorescence assay.

110. The method of any one of claims 95-109, wherein the GPI-AP deficient cells are PIGA null blood cells.

111. The method of claim 110, wherein the GPI-AP deficient cells are PIGA null lymphoblast TF1 (TF1PIGAnull) cells.

112. A method of assessing a risk of a patient with SARS-CoV-2 infection to develop a severe vascular complication, comprising:
(a) detecting the presence or absence of one or more mutations in complement proteins in a cell sample obtained from the patient; and
(b) identifying the patient as being at high risk for developing the vascular complication if the patient's cell sample comprises one or more mutations in complement proteins.

113. The method of claim 112, wherein the one or more mutations in complement proteins comprises a gain of function (GOF) mutation in a complement activating factor and/or a loss of function (LOF) mutation in a complement inhibitory factor.

114. The method of claim 113, wherein the GOF mutation in a complement activating factor is a GOF mutation in complement C3 and/or complement factor B; and
the LOF mutation in a complement inhibitory factor is a LOF mutation in one or more of complement factor H (CFH); a CFH-related protein selected from complement factor H receptor 1 (CFHR1), complement factor H receptor 2 (CFHR2), complement factor H receptor 3 (CFHR3), complement factor H receptor 1 (CFHR4), and complement factor H receptor 5 (CFHR5); complement factor I (CFI); membrane cofactor protein (MCP or CD46); thrombomodulin (THBD); and complement receptor 1 (CR1).

115. A method for treating a patient with a SARS-CoV-2 infection who is at risk for developing a severe vascular complications, comprising:
(a) identifying a patient as being at high risk for developing the vascular complications according to the method of any one of claims 112-114; and
(b) administering the patient a therapeutically effective amount of the CFD inhibitor for use of claim 94.

116. A method of screening for test agents that inhibit SARS-CoV-2 spike protein-mediated cell death, comprising:
(a) incubating GPI-AP deficient cells with human serum comprising recombinant SARS-CoV-2 spike proteins S1 and/or S2 for a period sufficient to induce APC on the GPI-AP cells to form an incubate;
(b) treating the incubate:
(i) with a test agent to produce a test incubate; and
(ii) without a test agent to produce a null incubate;
(c) measuring cell viability of the GPI-AP deficient cells in the test and null incubates; and
(d) comparing the cell viability of the GPI-AP deficient cells in the test incubate to the cell viability in the null incubate;
wherein an increase in the cell viability in the test incubate compared to the cell viability in the null incubate indicates that the test agent is capable of inhibiting SARS-CoV-2 mediated cell death.

117. The method of claim 116, wherein the one or more control agent comprises an agent which effectively blocks the binding of SARS-CoV-2 spike proteins to cell surface.

118. The method of claim 117, wherein the agent which effectively blocks the binding of SARS-CoV-2 spike proteins to cell surface is heparan sulfate or a derivative thereof.

119. The method of any one of claims 116-118, further comprising determining deposition of complement proteins C5b9, C4d, and/or C3c on the surface of GPI-AP deficient cells in the null sample and the test sample.

120. The method of any one of claims 116-119, wherein step (b) further comprises (iii) treating the incubate with one or more control agents to produce one or more control incubates, and wherein the method further comprises (e) comparing the cell viability of GPI-AP deficient cells in the test incubate to the cell viability of the GPI-AP deficient cells in the one or more control incubates, wherein an increase in the cell viability in the test incubate compared to the cell viability in the one or more control incubates indicates that the test agent is capable of inhibiting SARS-CoV-2 spike-protein-mediated cell death.

121. The method of claim 120, wherein the cell viability of GPI-AP cells in the one or more control incubates is higher than that of GPI-AP cells in the null incubate.

122. The method of claim 120 or 121, further comprising determining deposition of complement proteins C5b9, C4d, and/or C3c on the surface of GPI-AP deficient cells in the one or more control samples.

123. The method of claim 119 or 122, wherein the deposition of complement proteins is measured using a fluorescence assay.

124. The method of any one of claims 116-123, wherein cell viability is measured using a fluorescence assay.

125. The method of any one of claims 116-124, wherein the GPI-AP deficient cells are PIGA null blood cells.

126. The method of claim 125, wherein the GPI-AP deficient cells are TF1PIGAnull cells.
